SEQUENCE LISTING (73 sequences)

SEQ ID NO: 1 - DNA, 4853 bp, Homo sapiens
  misc_feature (70)..(3489): CDS
ttcgccgtttgaattgctgcgggcccgggccctcacctcacctgaggtccggccgcccaggggtgcgctatgccgtcgggaggtgaccagtcgccaccgcccccgcctccccctccggcggcggcagcctcggatgaggaggaggaggacgacggcgaggcggaagacgccgcgccgtctgccgagtcgcccacccctcagatccagcagcggttcgacgagctgtgcagccgcctcaacatggacgaggcggcgcggcccgaggcctgggacagctaccgcagcatgagcgaaagctacacgctggagggaaatgatcttcattggttagcatgtgccttatatgtggcttgcagaaaatctgttccaactgtaagcaaagggacagtggaaggaaactatgtatctttaactagaatcctgaaatgttcagagcagagcttaatcgaattttttaataagatgaagaagtgggaagacatggcaaatctacccccacatttcagagaacgtactgagagattagaaagaaacttcactgtttctgctgtaatttttaagaaatatgaacccatttttcaggacatctttaaataccctcaagaggagcaacctcgtcagcagcgaggaaggaaacagcggcgacagccctgtactgtgtctgaaattttccatttttgttgggtgctttttatatatgcaaaaggtaatttccccatgattagtgatgatttggtcaattcttatcacctgctgctgtgtgctttggacttagtttatggaaatgcacttcagtgttctaatcgtaaagaacttgtgaaccctaattttaaaggcttatctgaagattttcatgctaaagattctaaaccttcctctgaccccccttgtatcattgagaaactgtgttccttacatgatggcctagttttggaagcaaaggggataaaggaacatttctggaaaccctatattaggaaactttatgaaaaaaagctccttaagggaaaagaagaaaatctcactgggtttctagaacctgggaactttggagagagttttaaagccatcaataaggcctatgaggagtatgttttatctgttgggaatttagatgagcggatatttcttggagaggatgctgaggaggaaattgggactctctcaaggtgtctgaacgctggttcaggaacagagactgctgaaagggtgcagatgaaaaacatcttacagcagcattttgacaagtccaaagcacttagaatctccacaccactaactggtgttaggtacattaaggagaatagcccttgtgtgactccagtttctacagctacgcatagcttgagtcgtcttcacaccatgctgacaggcctcaggaatgcaccaagtgagaaactggaacagattctcaggacatgttccagagatccaacccaggctattgctaacagactgaaagaaatgtttgaaatatattctcagcatttccagccagacgaggatttcagtaattgtgctaaagaaattgccagcaaacattttcgttttgcggagatgctttactataaagtattagaatctgttattgagcaggaacaaaaaagactaggagacatggatttatctggtattctggaacaagatgcattccacagatctctcttggcctgctgccttgaggtcgtcactttttcttataagcctcctgggaattttccatttattactgaaatatttgatgtgcctctttatcatttttataaggtgatagaagtattcattagagcagaagatggcctttgtagagaggtggtaaaacaccttaatcagattgaagaacagatcttagatcatttggcatggaaaccagagtctccactctgggaaaaaattagagacaatgaaaacagagttcctacatgtgaagaggtcatgccacctcagaacctggaaagggcagatgaaatttgcattgctggctcccctttgactcccagaagggtgactgaagttcgtgctgatactggaggacttggaaggagcataacatctccaaccacattatacgataggtacagctccccaccagccagcactaccagaaggcggctatttgttgagaatgatagcccctctgatggagggacgcctgggcgcatgcccccacagcccctagtcaatgctgtccctgtgcagaatgtatctggggagactgtttctgtcacaccagttcctggacagactttggtcaccatggcaaccgccactgtcacagccaacaatgggcaaacggtaaccattcctgtgcaaggtattgccaatgaaaatggagggataacattcttccctgtccaagtcaatgttggggggcaggcacaagctgtgacaggctccatccagcccctcagtgctcaggccctggctggaagtctgagctctcaacaggtgacaggaacaactttgcaagtccctggtcaagtggccattcaacagatttccccaggtggccaacagcagaagcaaggccagtctgtaaccagcagtagtaatagacccaggaagaccagctctttatcgcttttctttagaaaggtataccatttagcagctgtccgccttcgggatctctgtgccaaactagatatttcagatgaattgaggaaaaaaatctggacctgctttgaattctccataattcagtgtcctgaacttatgatggacagacatctggaccagttattaatgtgtgccatttatgtgatggcaaaggtcacaaaagaagataagtccttccagaacattatgcgttgttataggactcagccgcaggcccggagccaggtgtatagaagtgttttgataaaagggaaaagaaaaagaagaaattctggcagcagtgatagcagaagccatcagaattctccaacagaactaaacaaagatagaaccagtagagactccagtccagttatgaggtcaagcagcaccttgccagttccacagcccagcagtgctcctcccacacctactcgcctcacaggtgccaacagtgacatggaagaagaggagaggggagacctcattcagttctacaacaacatctacatcaaacagattaagacatttgccatgaagtactcacaggcaaatatggatgctcctccactctctccctatccatttgtaagaacaggctcccctcgccgaatacagttgtctcaaaatcatcctgtctacatttccccacataaaaatgaaacaatgctttctcctcgagaaaagattttctattacttcagcaacagtccttcaaagagactgagagaaattaatagtatgatacgcacaggagaaactcctactaaaaagagaggaattcttttggaagatggaagtgaatcacctgcaaaaagaatttgcccagaaaatcattctgccttattacgccgtctccaagatgtagctaatgaccgtggttcccactgaggttagtctcttgtattaaactcttcacaaaatctgtttagcagcagcctttaatgcatctagattatggagcttttttccttaatccagctgatgagttacagcctgttagtaacatgaggggacattttggtgagaaatgggacttaactccttccagtgtccttagaacattttaattcatcccaactgtctttttttccctaccactcagtgattactgtcaaggctgcttacaatccaaacttgggtttttggctctggcaaagcttttagaaatactgcaagaaatgatgtgtacccaacgtgagcataggaggcttctgttgacgtctccaacagaagaactgtgtttcaagttcaatcctacctgttttgtggtcagctgtagtcctcataaaaagcaaaacaaaaattaggtattttgtcctaaaacacctggtaggagtgtgtgattttttgcattcctgacaaaggagagcacacccaggtttggaggtcctaggtcattagccctcgtctcccgttccctttgtgcacatcttccctctccccattcggtgtggtgcagtgtgaaaagtccttgattgttcgggtgtgcaatgtctgagtgaacctgtataagtggaggcactttagggctgtaaaatgcatgattttgtaacccagattttgctgtatatttgtgatagcactttctacaatgtgaactttattaaatacaaaacttccaggctaaacatccaatattttctttaatgcttttatatttttttaaaatgttaaaacccctatagccaccttttgggaatgttttaaattctccagttttttgttatatagggatcaaccagctaagaaaagattttaagtcaagttgaattgaggggattaatatgaaaacttatgacctcttcctttaggagggagttatctaaaagaaatgtctattaaggtgatatatttaaaaatatttttgggtgttcctggcagtttaaaaaaattggttggagaatttaggtttttattagtaccatagtaccatttatacaaattagaaaatgttatttaacagctgaattatctatacatatctttattaatcactattgttccagcagttttcaagtcaaattaataatcttattagggagaaaattcaattgtaaattgaatcagtataaacaaagttactaggtaacttcatattgctgagagaaatatggaacttacattgttcaattagaatagtgttctccccaaatatttataaaacttctcaagatactgctacgtgtaattttatatgaagataagtgtatttttcaataaagcatttataaattaaaaaaaaaaaaaaaaaa

SEQ ID NO: 2 - protein, 1139 aa, Homo sapiens
MPSGGDQSPPPPPPPPAAAASDEEEEDDGEAEDAAPSAESPTPQIQQRFDELCSRLNMDEAARPEAWDSYRSMSESYTLEGNDLHWLACALYVACRKSVPTVSKGTVEGNYVSLTRILKCSEQSLIEFFNKMKKWEDMANLPPHFRERTERLERNFTVSAVIFKKYEPIFQDIFKYPQEEQPRQQRGRKQRRQPCTVSEIFHFCWVLFIYAKGNFPMISDDLVNSYHLLLCALDLVYGNALQCSNRKELVNPNFKGLSEDFHAKDSKPSSDPPCIIEKLCSLHDGLVLEAKGIKEHFWKPYIRKLYEKKLLKGKEENLTGFLEPGNFGESFKAINKAYEEYVLSVGNLDERIFLGEDAEEEIGTLSRCLNAGSGTETAERVQMKNILQQHFDKSKALRISTPLTGVRYIKENSPCVTPVSTATHSLSRLHTMLTGLRNAPSEKLEQILRTCSRDPTQAIANRLKEMFEIYSQHFQPDEDFSNCAKEIASKHFRFAEMLYYKVLESVIEQEQKRLGDMDLSGILEQDAFHRSLLACCLEVVTFSYKPPGNFPFITEIFDVPLYHFYKVIEVFIRAEDGLCREVVKHLNQIEEQILDHLAWKPESPLWEKIRDNENRVPTCEEVMPPQNLERADEICIAGSPLTPRRVTEVRADTGGLGRSITSPTTLYDRYSSPPASTTRRRLFVENDSPSDGGTPGRMPPQPLVNAVPVQNVSGETVSVTPVPGQTLVTMATATVTANNGQTVTIPVQGIANENGGITFFPVQVNVGGQAQAVTGSIQPLSAQALAGSLSSQQVTGTTLQVPGQVAIQQISPGGQQQKQGQSVTSSSNRPRKTSSLSLFFRKVYHLAAVRLRDLCAKLDISDELRKKIWTCFEFSIIQCPELMMDRHLDQLLMCAIYVMAKVTKEDKSFQNIMRCYRTQPQARSQVYRSVLIKGKRKRRNSGSSDSRSHQNSPTELNKDRTSRDSSPVMRSSSTLPVPQPSSAPPTPTRLTGANSDMEEEERGDLIQFYNNIYIKQIKTFAMKYSQANMDAPPLSPYPFVRTGSPRRIQLSQNHPVYISPHKNETMLSPREKIFYYFSNSPSKRLREINSMIRTGETPTKKRGILLEDGSESPAKRICPENHSALLRRLQDVANDRGSH

SEQ ID NO: 3 - DNA, 4853 bp, Homo sapiens
  misc_feature (70)..(3489): CDS
ttcgccgtttgaattgctgcgggcccgggccctcacctcacctgaggtccggccgcccaggggtgcgctatgccgtcgggaggtgaccagtcgccaccgcccccgcctccccctccggcggcggcagcctcggatgaggaggaggaggacgacggcgaggcggaagacgccgcgccgcctgccgagtcgcccacccctcagatccagcagcggttcgacgagctgtgcagccgcctcaacatggacgaggcggcgcggcccgaggcctgggacagctaccgcagcatgagcgaaagctacacgctggagggaaatgatcttcattggttagcatgtgccttatatgtggcttgcagaaaatctgttccaactgtaagcaaagggacagtggaaggaaactatgtatctttaactagaatcctgaaatgttcagagcagagcttaatcgaattttttaataagatgaagaagtgggaagacatggcaaatctacccccacatttcagagaacgtactgagagattagaaagaaacttcactgtttctgctgtaatttttaagaaatatgaacccatttttcaggacatctttaaataccctcaagaggagcaacctcgtcagcagcgaggaaggaaacagcggcgacagccctgtactgtgtctgaaattttccatttttgttgggtgctttttatatatgcaaaaggtaatttccccatgattagtgatgatttggtcaattcttatcacctgctgctgtgtgctttggacttagtttatggaaatgcacttcagtgttctaatcgtaaagaacttgtgaaccctaattttaaaggcttatctgaagattttcatgctaaagattctaaaccttcctctgaccccccttgtatcattgagaaactgtgttccttacatgatggcctagttttggaagcaaaggggataaaggaacatttctggaaaccctatattaggaaactttatgaaaaaaagctccttaagggaaaagaagaaaatctcactgggtttctagaacctgggaactttggagagagttttaaagccatcaataaggcctatgaggagtatgttttatctgttgggaatttagatgagcggatatttcttggagaggatgctgaggaggaaattgggactctctcaaggtgtctgaacgctggttcaggaacagagactgctgaaagggtgcagatgaaaaacatcttacagcagcattttgacaagtccaaagcacttagaatctccacaccactaactggtgttaggtacattaaggagaatagcccttgtgtgactccagtttctacagctacgcatagcttgagtcgtcttcacaccatgctgacaggcctcaggaatgcaccaagtgagaaactggaacagattctcaggacatgttccagagatccaacccaggctattgctaacagactgaaagaaatgtttgaaatatattctcagcatttccagccagacgaggatttcagtaattgtgctaaagaaattgccagcaaacattttcgttttgcggagatgctttactataaagtattagaatctgttattgagcaggaacaaaaaagactaggagacatggatttatctggtattctggaacaagatgcattccacagatctctcttggcctgctgccttgaggtcgtcactttttcttataagcctcctgggaattttccatttattactgaaatatttgatgtgcctctttatcatttttataaggtgatagaagtattcattagagcagaagatggcctttgtagagaggtggtaaaacaccttaatcagattgaagaacagatcttagatcatttggcatggaaaccagagtctccactctgggaaaaaattagagacaatgaaaacagagttcctacatgtgaagaggtcatgccacctcagaacctggaaagggcagatgaaatttgcattgctggctcccctttgactcccagaagggtgactgaagttcgtgctgatactggaggacttggaaggagcataacatctccaaccacattatacgataggtacagctccccaccagccagcactaccagaaggcggctatttgttgagaatgatagcccctctgatggagggacgcctgggcgcatgcccccacagcccctagtcaatgctgtccctgtgcagaatgtatctggggagactgtttctgtcacaccagttcctggacagactttggtcaccatggcaaccgccactgtcacagccaacaatgggcaaacggtaaccattcctgtgcaaggtattgccaatgaaaatggagggataacattcttccctgtccaagtcaatgttggggggcaggcacaagctgtgacaggctccatccagcccctcagtgctcaggccctggctggaagtctgagctctcaacaggtgacaggaacaactttgcaagtccctggtcaagtggccattcaacagatttccccaggtggccaacagcagaagcaaggccagtctgtaaccagcagtagtaatagacccaggaagaccagctctttatcgcttttctttagaaaggtataccatttagcagctgtccgccttcgggatctctgtgccaaactagatatttcagatgaattgaggaaaaaaatctggacctgctttgaattctccataattcagtgtcctgaacttatgatggacagacatctggaccagttattaatgtgtgccatttatgtgatggcaaaggtcacaaaagaagataagtccttccagaacattatgcgttgttataggactcagccgcaggcccggagccaggtgtatagaagtgttttgataaaagggaaaagaaaaagaagaaattctggcagcagtgatagcagaagccatcagaattctccaacagaactaaacaaagatagaaccagtagagactccagtccagttatgaggtcaagcagcaccttgccagttccacagcccagcagtgctcctcccacacctactcgcctcacaggtgccaacagtgacatggaagaagaggagaggggagacctcattcagttctacaacaacatctacatcaaacagattaagacatttgccatgaagtactcacaggcaaatatggatgctcctccactctctccctatccatttgtaagaacaggctcccctcgccgaatacagttgtctcaaaatcatcctgtctacatttccccacataaaaatgaaacaatgctttctcctcgagaaaagattttctattacttcagcaacagtccttcaaagagactgagagaaattaatagtatgatacgcacaggagaaactcctactaaaaagagaggaattcttttggaagatggaagtgaatcacctgcaaaaagaatttgcccagaaaatcattctgccttattacgccgtctccaagatgtagctaatgaccgtggttcccactgaggttagtctcttgtattaaactcttcacaaaatctgtttagcagcagcctttaatgcatctagattatggagcttttttccttaatccagctgatgagttacagcctgttagtaacatgaggggacattttggtgagaaatgggacttaactccttccagtgtccttagaacattttaattcatcccaactgtctttttttccctaccactcagtgattactgtcaaggctgcttacaatccaaacttgggtttttggctctggcaaagcttttagaaatactgcaagaaatgatgtgtacccaacgtgagcataggaggcttctgttgacgtctccaacagaagaactgtgtttcaagttcaatcctacctgttttgtggtcagctgtagtcctcataaaaagcaaaacaaaaattaggtattttgtcctaaaacacctggtaggagtgtgtgattttttgcattcctgacaaaggagagcacacccaggtttggaggtcctaggtcattagccctcgtctcccgttccctttgtgcacatcttccctctccccattcggtgtggtgcagtgtgaaaagtccttgattgttcgggtgtgcaatgtctgagtgaacctgtataagtggaggcactttagggctgtaaaatgcatgattttgtaacccagattttgctgtatatttgtgatagcactttctacaatgtgaactttattaaatacaaaacttccaggctaaacatccaatattttctttaatgcttttatatttttttaaaatgttaaaacccctatagccaccttttgggaatgttttaaattctccagttttttgttatatagggatcaaccagctaagaaaagattttaagtcaagttgaattgaggggattaatatgaaaacttatgacctcttcctttaggagggagttatctaaaagaaatgtctattaaggtgatatatttaaaaatatttttgggtgttcctggcagtttaaaaaaattggttggagaatttaggtttttattagtaccatagtaccatttatacaaattagaaaatgttatttaacagctgaattatctatacatatctttattaatcactattgttccagcagttttcaagtcaaattaataatcttattagggagaaaattcaattgtaaattgaatcagtataaacaaagttactaggtaacttcatattgctgagagaaatatggaacttacattgttcaattagaatagtgttctccccaaatatttataaaacttctcaagatactgctacgtgtaattttatatgaagataagtgtatttttcaataaagcatttataaattaaaaaaaaaaaaaaaaaa

SEQ ID NO: 4 - protein, 1139 aa, Homo sapiens
  VARIANT (37)..(37): Ser to Pro
MPSGGDQSPPPPPPPPAAAASDEEEEDDGEAEDAAPPAESPTPQIQQRFDELCSRLNMDEAARPEAWDSYRSMSESYTLEGNDLHWLACALYVACRKSVPTVSKGTVEGNYVSLTRILKCSEQSLIEFFNKMKKWEDMANLPPHFRERTERLERNFTVSAVIFKKYEPIFQDIFKYPQEEQPRQQRGRKQRRQPCTVSEIFHFCWVLFIYAKGNFPMISDDLVNSYHLLLCALDLVYGNALQCSNRKELVNPNFKGLSEDFHAKDSKPSSDPPCIIEKLCSLHDGLVLEAKGIKEHFWKPYIRKLYEKKLLKGKEENLTGFLEPGNFGESFKAINKAYEEYVLSVGNLDERIFLGEDAEEEIGTLSRCLNAGSGTETAERVQMKNILQQHFDKSKALRISTPLTGVRYIKENSPCVTPVSTATHSLSRLHTMLTGLRNAPSEKLEQILRTCSRDPTQAIANRLKEMFEIYSQHFQPDEDFSNCAKEIASKHFRFAEMLYYKVLESVIEQEQKRLGDMDLSGILEQDAFHRSLLACCLEVVTFSYKPPGNFPFITEIFDVPLYHFYKVIEVFIRAEDGLCREVVKHLNQIEEQILDHLAWKPESPLWEKIRDNENRVPTCEEVMPPQNLERADEICIAGSPLTPRRVTEVRADTGGLGRSITSPTTLYDRYSSPPASTTRRRLFVENDSPSDGGTPGRMPPQPLVNAVPVQNVSGETVSVTPVPGQTLVTMATATVTANNGQTVTIPVQGIANENGGITFFPVQVNVGGQAQAVTGSIQPLSAQALAGSLSSQQVTGTTLQVPGQVAIQQISPGGQQQKQGQSVTSSSNRPRKTSSLSLFFRKVYHLAAVRLRDLCAKLDISDELRKKIWTCFEFSIIQCPELMMDRHLDQLLMCAIYVMAKVTKEDKSFQNIMRCYRTQPQARSQVYRSVLIKGKRKRRNSGSSDSRSHQNSPTELNKDRTSRDSSPVMRSSSTLPVPQPSSAPPTPTRLTGANSDMEEEERGDLIQFYNNIYIKQIKTFAMKYSQANMDAPPLSPYPFVRTGSPRRIQLSQNHPVYISPHKNETMLSPREKIFYYFSNSPSKRLREINSMIRTGETPTKKRGILLEDGSESPAKRICPENHSALLRRLQDVANDRGSH

SEQ ID NO: 5 - DNA, 4853 bp, Homo sapiens
  misc_feature (70)..(3489): CDS
ttcgccgtttgaattgctgcgggcccgggccctcacctcacctgaggtccggccgcccaggggtgcgctatgccgtcgggaggtgaccagtcgccaccgcccccgcctccccctccggcggcggcagcctcggatgaggaggaggaggacgacggcgaggcggaagacgccgcgccgtctgccgagtcgcccacccctcagatccagcagcggttcgacgagctgtgcagccgcctcaacatggacgaggcggcgcgggccgaggcctgggacagctaccgcagcatgagcgaaagctacacgctggagggaaatgatcttcattggttagcatgtgccttatatgtggcttgcagaaaatctgttccaactgtaagcaaagggacagtggaaggaaactatgtatctttaactagaatcctgaaatgttcagagcagagcttaatcgaattttttaataagatgaagaagtgggaagacatggcaaatctacccccacatttcagagaacgtactgagagattagaaagaaacttcactgtttctgctgtaatttttaagaaatatgaacccatttttcaggacatctttaaataccctcaagaggagcaacctcgtcagcagcgaggaaggaaacagcggcgacagccctgtactgtgtctgaaattttccatttttgttgggtgctttttatatatgcaaaaggtaatttccccatgattagtgatgatttggtcaattcttatcacctgctgctgtgtgctttggacttagtttatggaaatgcacttcagtgttctaatcgtaaagaacttgtgaaccctaattttaaaggcttatctgaagattttcatgctaaagattctaaaccttcctctgaccccccttgtatcattgagaaactgtgttccttacatgatggcctagttttggaagcaaaggggataaaggaacatttctggaaaccctatattaggaaactttatgaaaaaaagctccttaagggaaaagaagaaaatctcactgggtttctagaacctgggaactttggagagagttttaaagccatcaataaggcctatgaggagtatgttttatctgttgggaatttagatgagcggatatttcttggagaggatgctgaggaggaaattgggactctctcaaggtgtctgaacgctggttcaggaacagagactgctgaaagggtgcagatgaaaaacatcttacagcagcattttgacaagtccaaagcacttagaatctccacaccactaactggtgttaggtacattaaggagaatagcccttgtgtgactccagtttctacagctacgcatagcttgagtcgtcttcacaccatgctgacaggcctcaggaatgcaccaagtgagaaactggaacagattctcaggacatgttccagagatccaacccaggctattgctaacagactgaaagaaatgtttgaaatatattctcagcatttccagccagacgaggatttcagtaattgtgctaaagaaattgccagcaaacattttcgttttgcggagatgctttactataaagtattagaatctgttattgagcaggaacaaaaaagactaggagacatggatttatctggtattctggaacaagatgcattccacagatctctcttggcctgctgccttgaggtcgtcactttttcttataagcctcctgggaattttccatttattactgaaatatttgatgtgcctctttatcatttttataaggtgatagaagtattcattagagcagaagatggcctttgtagagaggtggtaaaacaccttaatcagattgaagaacagatcttagatcatttggcatggaaaccagagtctccactctgggaaaaaattagagacaatgaaaacagagttcctacatgtgaagaggtcatgccacctcagaacctggaaagggcagatgaaatttgcattgctggctcccctttgactcccagaagggtgactgaagttcgtgctgatactggaggacttggaaggagcataacatctccaaccacattatacgataggtacagctccccaccagccagcactaccagaaggcggctatttgttgagaatgatagcccctctgatggagggacgcctgggcgcatgcccccacagcccctagtcaatgctgtccctgtgcagaatgtatctggggagactgtttctgtcacaccagttcctggacagactttggtcaccatggcaaccgccactgtcacagccaacaatgggcaaacggtaaccattcctgtgcaaggtattgccaatgaaaatggagggataacattcttccctgtccaagtcaatgttggggggcaggcacaagctgtgacaggctccatccagcccctcagtgctcaggccctggctggaagtctgagctctcaacaggtgacaggaacaactttgcaagtccctggtcaagtggccattcaacagatttccccaggtggccaacagcagaagcaaggccagtctgtaaccagcagtagtaatagacccaggaagaccagctctttatcgcttttctttagaaaggtataccatttagcagctgtccgccttcgggatctctgtgccaaactagatatttcagatgaattgaggaaaaaaatctggacctgctttgaattctccataattcagtgtcctgaacttatgatggacagacatctggaccagttattaatgtgtgccatttatgtgatggcaaaggtcacaaaagaagataagtccttccagaacattatgcgttgttataggactcagccgcaggcccggagccaggtgtatagaagtgttttgataaaagggaaaagaaaaagaagaaattctggcagcagtgatagcagaagccatcagaattctccaacagaactaaacaaagatagaaccagtagagactccagtccagttatgaggtcaagcagcaccttgccagttccacagcccagcagtgctcctcccacacctactcgcctcacaggtgccaacagtgacatggaagaagaggagaggggagacctcattcagttctacaacaacatctacatcaaacagattaagacatttgccatgaagtactcacaggcaaatatggatgctcctccactctctccctatccatttgtaagaacaggctcccctcgccgaatacagttgtctcaaaatcatcctgtctacatttccccacataaaaatgaaacaatgctttctcctcgagaaaagattttctattacttcagcaacagtccttcaaagagactgagagaaattaatagtatgatacgcacaggagaaactcctactaaaaagagaggaattcttttggaagatggaagtgaatcacctgcaaaaagaatttgcccagaaaatcattctgccttattacgccgtctccaagatgtagctaatgaccgtggttcccactgaggttagtctcttgtattaaactcttcacaaaatctgtttagcagcagcctttaatgcatctagattatggagcttttttccttaatccagctgatgagttacagcctgttagtaacatgaggggacattttggtgagaaatgggacttaactccttccagtgtccttagaacattttaattcatcccaactgtctttttttccctaccactcagtgattactgtcaaggctgcttacaatccaaacttgggtttttggctctggcaaagcttttagaaatactgcaagaaatgatgtgtacccaacgtgagcataggaggcttctgttgacgtctccaacagaagaactgtgtttcaagttcaatcctacctgttttgtggtcagctgtagtcctcataaaaagcaaaacaaaaattaggtattttgtcctaaaacacctggtaggagtgtgtgattttttgcattcctgacaaaggagagcacacccaggtttggaggtcctaggtcattagccctcgtctcccgttccctttgtgcacatcttccctctccccattcggtgtggtgcagtgtgaaaagtccttgattgttcgggtgtgcaatgtctgagtgaacctgtataagtggaggcactttagggctgtaaaatgcatgattttgtaacccagattttgctgtatatttgtgatagcactttctacaatgtgaactttattaaatacaaaacttccaggctaaacatccaatattttctttaatgcttttatatttttttaaaatgttaaaacccctatagccaccttttgggaatgttttaaattctccagttttttgttatatagggatcaaccagctaagaaaagattttaagtcaagttgaattgaggggattaatatgaaaacttatgacctcttcctttaggagggagttatctaaaagaaatgtctattaaggtgatatatttaaaaatatttttgggtgttcctggcagtttaaaaaaattggttggagaatttaggtttttattagtaccatagtaccatttatacaaattagaaaatgttatttaacagctgaattatctatacatatctttattaatcactattgttccagcagttttcaagtcaaattaataatcttattagggagaaaattcaattgtaaattgaatcagtataaacaaagttactaggtaacttcatattgctgagagaaatatggaacttacattgttcaattagaatagtgttctccccaaatatttataaaacttctcaagatactgctacgtgtaattttatatgaagataagtgtatttttcaataaagcatttataaattaaaaaaaaaaaaaaaaaa

SEQ ID NO: 6 - protein, 1139 aa, Homo sapiens
  VARIANT (64)..(64): Pro to Ala mutation
MPSGGDQSPPPPPPPPAAAASDEEEEDDGEAEDAAPSAESPTPQIQQRFDELCSRLNMDEAARAEAWDSYRSMSESYTLEGNDLHWLACALYVACRKSVPTVSKGTVEGNYVSLTRILKCSEQSLIEFFNKMKKWEDMANLPPHFRERTERLERNFTVSAVIFKKYEPIFQDIFKYPQEEQPRQQRGRKQRRQPCTVSEIFHFCWVLFIYAKGNFPMISDDLVNSYHLLLCALDLVYGNALQCSNRKELVNPNFKGLSEDFHAKDSKPSSDPPCIIEKLCSLHDGLVLEAKGIKEHFWKPYIRKLYEKKLLKGKEENLTGFLEPGNFGESFKAINKAYEEYVLSVGNLDERIFLGEDAEEEIGTLSRCLNAGSGTETAERVQMKNILQQHFDKSKALRISTPLTGVRYIKENSPCVTPVSTATHSLSRLHTMLTGLRNAPSEKLEQILRTCSRDPTQAIANRLKEMFEIYSQHFQPDEDFSNCAKEIASKHFRFAEMLYYKVLESVIEQEQKRLGDMDLSGILEQDAFHRSLLACCLEVVTFSYKPPGNFPFITEIFDVPLYHFYKVIEVFIRAEDGLCREVVKHLNQIEEQILDHLAWKPESPLWEKIRDNENRVPTCEEVMPPQNLERADEICIAGSPLTPRRVTEVRADTGGLGRSITSPTTLYDRYSSPPASTTRRRLFVENDSPSDGGTPGRMPPQPLVNAVPVQNVSGETVSVTPVPGQTLVTMATATVTANNGQTVTIPVQGIANENGGITFFPVQVNVGGQAQAVTGSIQPLSAQALAGSLSSQQVTGTTLQVPGQVAIQQISPGGQQQKQGQSVTSSSNRPRKTSSLSLFFRKVYHLAAVRLRDLCAKLDISDELRKKIWTCFEFSIIQCPELMMDRHLDQLLMCAIYVMAKVTKEDKSFQNIMRCYRTQPQARSQVYRSVLIKGKRKRRNSGSSDSRSHQNSPTELNKDRTSRDSSPVMRSSSTLPVPQPSSAPPTPTRLTGANSDMEEEERGDLIQFYNNIYIKQIKTFAMKYSQANMDAPPLSPYPFVRTGSPRRIQLSQNHPVYISPHKNETMLSPREKIFYYFSNSPSKRLREINSMIRTGETPTKKRGILLEDGSESPAKRICPENHSALLRRLQDVANDRGSH

SEQ ID NO: 7 - DNA, 4853 bp, Homo sapiens
  misc_feature (70)..(3489): CDS
ttcgccgtttgaattgctgcgggcccgggccctcacctcacctgaggtccggccgcccaggggtgcgctatgccgtcgggaggtgaccagtcgccaccgcccccgcctccccctccggcggcggcagcctcggatgaggaggaggaggacgacggcgaggcggaagacgccgcgccgcctgccgagtcgcccacccctcagatccagcagcggttcgacgagctgtgcagccgcctcaacatggacgaggcggcgcgggccgaggcctgggacagctaccgcagcatgagcgaaagctacacgctggagggaaatgatcttcattggttagcatgtgccttatatgtggcttgcagaaaatctgttccaactgtaagcaaagggacagtggaaggaaactatgtatctttaactagaatcctgaaatgttcagagcagagcttaatcgaattttttaataagatgaagaagtgggaagacatggcaaatctacccccacatttcagagaacgtactgagagattagaaagaaacttcactgtttctgctgtaatttttaagaaatatgaacccatttttcaggacatctttaaataccctcaagaggagcaacctcgtcagcagcgaggaaggaaacagcggcgacagccctgtactgtgtctgaaattttccatttttgttgggtgctttttatatatgcaaaaggtaatttccccatgattagtgatgatttggtcaattcttatcacctgctgctgtgtgctttggacttagtttatggaaatgcacttcagtgttctaatcgtaaagaacttgtgaaccctaattttaaaggcttatctgaagattttcatgctaaagattctaaaccttcctctgaccccccttgtatcattgagaaactgtgttccttacatgatggcctagttttggaagcaaaggggataaaggaacatttctggaaaccctatattaggaaactttatgaaaaaaagctccttaagggaaaagaagaaaatctcactgggtttctagaacctgggaactttggagagagttttaaagccatcaataaggcctatgaggagtatgttttatctgttgggaatttagatgagcggatatttcttggagaggatgctgaggaggaaattgggactctctcaaggtgtctgaacgctggttcaggaacagagactgctgaaagggtgcagatgaaaaacatcttacagcagcattttgacaagtccaaagcacttagaatctccacaccactaactggtgttaggtacattaaggagaatagcccttgtgtgactccagtttctacagctacgcatagcttgagtcgtcttcacaccatgctgacaggcctcaggaatgcaccaagtgagaaactggaacagattctcaggacatgttccagagatccaacccaggctattgctaacagactgaaagaaatgtttgaaatatattctcagcatttccagccagacgaggatttcagtaattgtgctaaagaaattgccagcaaacattttcgttttgcggagatgctttactataaagtattagaatctgttattgagcaggaacaaaaaagactaggagacatggatttatctggtattctggaacaagatgcattccacagatctctcttggcctgctgccttgaggtcgtcactttttcttataagcctcctgggaattttccatttattactgaaatatttgatgtgcctctttatcatttttataaggtgatagaagtattcattagagcagaagatggcctttgtagagaggtggtaaaacaccttaatcagattgaagaacagatcttagatcatttggcatggaaaccagagtctccactctgggaaaaaattagagacaatgaaaacagagttcctacatgtgaagaggtcatgccacctcagaacctggaaagggcagatgaaatttgcattgctggctcccctttgactcccagaagggtgactgaagttcgtgctgatactggaggacttggaaggagcataacatctccaaccacattatacgataggtacagctccccaccagccagcactaccagaaggcggctatttgttgagaatgatagcccctctgatggagggacgcctgggcgcatgcccccacagcccctagtcaatgctgtccctgtgcagaatgtatctggggagactgtttctgtcacaccagttcctggacagactttggtcaccatggcaaccgccactgtcacagccaacaatgggcaaacggtaaccattcctgtgcaaggtattgccaatgaaaatggagggataacattcttccctgtccaagtcaatgttggggggcaggcacaagctgtgacaggctccatccagcccctcagtgctcaggccctggctggaagtctgagctctcaacaggtgacaggaacaactttgcaagtccctggtcaagtggccattcaacagatttccccaggtggccaacagcagaagcaaggccagtctgtaaccagcagtagtaatagacccaggaagaccagctctttatcgcttttctttagaaaggtataccatttagcagctgtccgccttcgggatctctgtgccaaactagatatttcagatgaattgaggaaaaaaatctggacctgctttgaattctccataattcagtgtcctgaacttatgatggacagacatctggaccagttattaatgtgtgccatttatgtgatggcaaaggtcacaaaagaagataagtccttccagaacattatgcgttgttataggactcagccgcaggcccggagccaggtgtatagaagtgttttgataaaagggaaaagaaaaagaagaaattctggcagcagtgatagcagaagccatcagaattctccaacagaactaaacaaagatagaaccagtagagactccagtccagttatgaggtcaagcagcaccttgccagttccacagcccagcagtgctcctcccacacctactcgcctcacaggtgccaacagtgacatggaagaagaggagaggggagacctcattcagttctacaacaacatctacatcaaacagattaagacatttgccatgaagtactcacaggcaaatatggatgctcctccactctctccctatccatttgtaagaacaggctcccctcgccgaatacagttgtctcaaaatcatcctgtctacatttccccacataaaaatgaaacaatgctttctcctcgagaaaagattttctattacttcagcaacagtccttcaaagagactgagagaaattaatagtatgatacgcacaggagaaactcctactaaaaagagaggaattcttttggaagatggaagtgaatcacctgcaaaaagaatttgcccagaaaatcattctgccttattacgccgtctccaagatgtagctaatgaccgtggttcccactgaggttagtctcttgtattaaactcttcacaaaatctgtttagcagcagcctttaatgcatctagattatggagcttttttccttaatccagctgatgagttacagcctgttagtaacatgaggggacattttggtgagaaatgggacttaactccttccagtgtccttagaacattttaattcatcccaactgtctttttttccctaccactcagtgattactgtcaaggctgcttacaatccaaacttgggtttttggctctggcaaagcttttagaaatactgcaagaaatgatgtgtacccaacgtgagcataggaggcttctgttgacgtctccaacagaagaactgtgtttcaagttcaatcctacctgttttgtggtcagctgtagtcctcataaaaagcaaaacaaaaattaggtattttgtcctaaaacacctggtaggagtgtgtgattttttgcattcctgacaaaggagagcacacccaggtttggaggtcctaggtcattagccctcgtctcccgttccctttgtgcacatcttccctctccccattcggtgtggtgcagtgtgaaaagtccttgattgttcgggtgtgcaatgtctgagtgaacctgtataagtggaggcactttagggctgtaaaatgcatgattttgtaacccagattttgctgtatatttgtgatagcactttctacaatgtgaactttattaaatacaaaacttccaggctaaacatccaatattttctttaatgcttttatatttttttaaaatgttaaaacccctatagccaccttttgggaatgttttaaattctccagttttttgttatatagggatcaaccagctaagaaaagattttaagtcaagttgaattgaggggattaatatgaaaacttatgacctcttcctttaggagggagttatctaaaagaaatgtctattaaggtgatatatttaaaaatatttttgggtgttcctggcagtttaaaaaaattggttggagaatttaggtttttattagtaccatagtaccatttatacaaattagaaaatgttatttaacagctgaattatctatacatatctttattaatcactattgttccagcagttttcaagtcaaattaataatcttattagggagaaaattcaattgtaaattgaatcagtataaacaaagttactaggtaacttcatattgctgagagaaatatggaacttacattgttcaattagaatagtgttctccccaaatatttataaaacttctcaagatactgctacgtgtaattttatatgaagataagtgtatttttcaataaagcatttataaattaaaaaaaaaaaaaaaaaa

SEQ ID NO: 8 - protein, 1139 aa, Homo sapiens
  VARIANT (37)..(37): Ser to Pro mutation
MPSGGDQSPPPPPPPPAAAASDEEEEDDGEAEDAAPPAESPTPQIQQRFDELCSRLNMDEAARAEAWDSYRSMSESYTLEGNDLHWLACALYVACRKSVPTVSKGTVEGNYVSLTRILKCSEQSLIEFFNKMKKWEDMANLPPHFRERTERLERNFTVSAVIFKKYEPIFQDIFKYPQEEQPRQQRGRKQRRQPCTVSEIFHFCWVLFIYAKGNFPMISDDLVNSYHLLLCALDLVYGNALQCSNRKELVNPNFKGLSEDFHAKDSKPSSDPPCIIEKLCSLHDGLVLEAKGIKEHFWKPYIRKLYEKKLLKGKEENLTGFLEPGNFGESFKAINKAYEEYVLSVGNLDERIFLGEDAEEEIGTLSRCLNAGSGTETAERVQMKNILQQHFDKSKALRISTPLTGVRYIKENSPCVTPVSTATHSLSRLHTMLTGLRNAPSEKLEQILRTCSRDPTQAIANRLKEMFEIYSQHFQPDEDFSNCAKEIASKHFRFAEMLYYKVLESVIEQEQKRLGDMDLSGILEQDAFHRSLLACCLEVVTFSYKPPGNFPFITEIFDVPLYHFYKVIEVFIRAEDGLCREVVKHLNQIEEQILDHLAWKPESPLWEKIRDNENRVPTCEEVMPPQNLERADEICIAGSPLTPRRVTEVRADTGGLGRSITSPTTLYDRYSSPPASTTRRRLFVENDSPSDGGTPGRMPPQPLVNAVPVQNVSGETVSVTPVPGQTLVTMATATVTANNGQTVTIPVQGIANENGGITFFPVQVNVGGQAQAVTGSIQPLSAQALAGSLSSQQVTGTTLQVPGQVAIQQISPGGQQQKQGQSVTSSSNRPRKTSSLSLFFRKVYHLAAVRLRDLCAKLDISDELRKKIWTCFEFSIIQCPELMMDRHLDQLLMCAIYVMAKVTKEDKSFQNIMRCYRTQPQARSQVYRSVLIKGKRKRRNSGSSDSRSHQNSPTELNKDRTSRDSSPVMRSSSTLPVPQPSSAPPTPTRLTGANSDMEEEERGDLIQFYNNIYIKQIKTFAMKYSQANMDAPPLSPYPFVRTGSPRRIQLSQNHPVYISPHKNETMLSPREKIFYYFSNSPSKRLREINSMIRTGETPTKKRGILLEDGSESPAKRICPENHSALLRRLQDVANDRGSH

SEQ ID NO: 9 - DNA, 4853 bp, Homo sapiens
  misc_feature (70)..(3489): CDS
ttcgccgtttgaattgctgcgggcccgggccctcacctcacctgaggtccggccgcccaggggtgcgctatgccgtcgggaggtgaccagtcgccaccgcccccgcctccccctccggcggcggcagcctcggatgaggaggaggaggacgacggcgaggcggaagacgccgcgccgtctgccgagtcgcccacccctcagatccagcagcggttcgacgagctgtgcagccgcctcaacatggacgaggcggcgcggcccgaggcctgggacagctaccgcagcatgagcgaaagctacacgctggagggaaatgatcttcattggttagcatgtgccttatatgtggcttgcagaaaatctgttccaactgtaagcaaagggacagtggaaggaaactatgtatctttaactagaatcctgaaatgttcagagcagagcttaatcgaattttttaataagatgaagaagtgggaagacatggcaaatctacccccacatttcagagaacgtactgagagattagaaagaaacttcactgtttctgctgtaatttttaagaaatatgaacccatttttcaggacatctttaaataccctcaagaggagcaacctcgtcagcagcgaggaaggaaacagcggcgacagccctgtactgtgtctgaaattttccatttttgttgggtgctttttatatatgcaaaaggtaatttccccatgattagtgatgatttggtcaattcttatcacctgctgctgtgtgctttggacttagtttatggaaatgcacttcagtgttctaatcgtaaagaacttgtgaaccctaattttaaaggcttatctgaagattttcatgctaaagattctaaaccttcctctgaccccccttgtatcattgagaaactgtgttccttacatgatggcctagttttggaagcaaaggggataaaggaacatttctggaaaccctatattaggaaactttatgaaaaaaagctccttaagggaaaagaagaaaatctcactgggtttctagaacctgggaactttggagagagttttaaagccatcaataaggcctatgaggagtatgttttatctgttgggaatttagatgagcggatatttcttggagaggatgctgaggaggaaattgggactctctcaaggtgtctgaacgctggttcaggaacagagactgctgaaagggtgcagatgaaaaacatcttacagcagcattttgacaagtccaaagcacttagaatctccacaccactaactggtgttaggtacattaaggagaatagcccttgtgtgactccagtttctacagctacgcatagcttgagtcgtcttcacaccatgctgacaggcctcaggaatgcaccaagtgagaaactggaacagattctcaggacatgttccagagatccaacccaggctattgctaacagactgaaagaaatgtttgaaatatattctcagcatttccagccagacgaggatttcagtaattgtgctaaagaaattgccagcaaacattttcgttttgcggagatgctttactataaagtattagaatctgttattgagcaggaacaaaaaagactaggagacatggatttatctggtattctggaacaagatgcgttccacagatctctcttggcctgctgccttgaggtcgtcactttttcttataagcctcctgggaattttccatttattactgaaatatttgatgtgcctctttatcatttttataaggtgatagaagtattcattagagcagaagatggcctttgtagagaggtggtaaaacaccttaatcagattgaagaacagatcttagatcatttggcatggaaaccagagtctccactctgggaaaaaattagagacaatgaaaacagagttcctacatgtgaagaggtcatgccacctcagaacctggaaagggcagatgaaatttgcattgctggctcccctttgactcccagaagggtgactgaagttcgtgctgatactggaggacttggaaggagcataacatctccaaccacattatacgataggtacagctccccaccagccagcactaccagaaggcggctatttgttgagaatgatagcccctctgatggagggacgcctgggcgcatgcccccacagcccctagtcaatgctgtccctgtgcagaatgtatctggggagactgtttctgtcacaccagttcctggacagactttggtcaccatggcaaccgccactgtcacagccaacaatgggcaaacggtaaccattcctgtgcaaggtattgccaatgaaaatggagggataacattcttccctgtccaagtcaatgttggggggcaggcacaagctgtgacaggctccatccagcccctcagtgctcaggccctggctggaagtctgagctctcaacaggtgacaggaacaactttgcaagtccctggtcaagtggccattcaacagatttccccaggtggccaacagcagaagcaaggccagtctgtaaccagcagtagtaatagacccaggaagaccagctctttatcgcttttctttagaaaggtataccatttagcagctgtccgccttcgggatctctgtgccaaactagatatttcagatgaattgaggaaaaaaatctggacctgctttgaattctccataattcagtgtcctgaacttatgatggacagacatctggaccagttattaatgtgtgccatttatgtgatggcaaaggtcacaaaagaagataagtccttccagaacattatgcgttgttataggactcagccgcaggcccggagccaggtgtatagaagtgttttgataaaagggaaaagaaaaagaagaaattctggcagcagtgatagcagaagccatcagaattctccaacagaactaaacaaagatagaaccagtagagactccagtccagttatgaggtcaagcagcaccttgccagttccacagcccagcagtgctcctcccacacctactcgcctcacaggtgccaacagtgacatggaagaagaggagaggggagacctcattcagttctacaacaacatctacatcaaacagattaagacatttgccatgaagtactcacaggcaaatatggatgctcctccactctctccctatccatttgtaagaacaggctcccctcgccgaatacagttgtctcaaaatcatcctgtctacatttccccacataaaaatgaaacaatgctttctcctcgagaaaagattttctattacttcagcaacagtccttcaaagagactgagagaaattaatagtatgatacgcacaggagaaactcctactaaaaagagaggaattcttttggaagatggaagtgaatcacctgcaaaaagaatttgcccagaaaatcattctgccttattacgccgtctccaagatgtagctaatgaccgtggttcccactgaggttagtctcttgtattaaactcttcacaaaatctgtttagcagcagcctttaatgcatctagattatggagcttttttccttaatccagctgatgagttacagcctgttagtaacatgaggggacattttggtgagaaatgggacttaactccttccagtgtccttagaacattttaattcatcccaactgtctttttttccctaccactcagtgattactgtcaaggctgcttacaatccaaacttgggtttttggctctggcaaagcttttagaaatactgcaagaaatgatgtgtacccaacgtgagcataggaggcttctgttgacgtctccaacagaagaactgtgtttcaagttcaatcctacctgttttgtggtcagctgtagtcctcataaaaagcaaaacaaaaattaggtattttgtcctaaaacacctggtaggagtgtgtgattttttgcattcctgacaaaggagagcacacccaggtttggaggtcctaggtcattagccctcgtctcccgttccctttgtgcacatcttccctctccccattcggtgtggtgcagtgtgaaaagtccttgattgttcgggtgtgcaatgtctgagtgaacctgtataagtggaggcactttagggctgtaaaatgcatgattttgtaacccagattttgctgtatatttgtgatagcactttctacaatgtgaactttattaaatacaaaacttccaggctaaacatccaatattttctttaatgcttttatatttttttaaaatgttaaaacccctatagccaccttttgggaatgttttaaattctccagttttttgttatatagggatcaaccagctaagaaaagattttaagtcaagttgaattgaggggattaatatgaaaacttatgacctcttcctttaggagggagttatctaaaagaaatgtctattaaggtgatatatttaaaaatatttttgggtgttcctggcagtttaaaaaaattggttggagaatttaggtttttattagtaccatagtaccatttatacaaattagaaaatgttatttaacagctgaattatctatacatatctttattaatcactattgttccagcagttttcaagtcaaattaataatcttattagggagaaaattcaattgtaaattgaatcagtataaacaaagttactaggtaacttcatattgctgagagaaatatggaacttacattgttcaattagaatagtgttctccccaaatatttataaaacttctcaagatactgctacgtgtaattttatatgaagataagtgtatttttcaataaagcatttataaattaaaaaaaaaaaaaaaaaa

SEQ ID NO: 10 - DNA, 18 bp, Artificial Sequence
  Synthetic primer
cctcacctcacctgaggt

SEQ ID NO: 11 - DNA, 20 bp, Artificial Sequence
  Synthetic primer
accggttcacaccaactagg

SEQ ID NO: 12 - DNA, 22 bp, Artificial Sequence
  Synthetic primer
gagatagggtcatcattgaaac

SEQ ID NO: 13 - DNA, 21 bp, Artificial Sequence
  Synthetic primer
cattagccatactctacttgt

SEQ ID NO: 14 - DNA, 22 bp, Artificial Sequence
  Synthetic primer
agctagtcagagacatgagttg

SEQ ID NO: 15 - DNA, 23 bp, Artificial Sequence
  Synthetic primer
cactgcagcacagactaatgtgt

SEQ ID NO: 16 - DNA, 23 bp, Artificial Sequence
  Synthetic primer
tctctccctttaactgtgggttt

SEQ ID NO: 17 - DNA, 23 bp, Artificial Sequence
  Synthetic primer
ggagttgacgagattaatacctg

SEQ ID NO: 18 - DNA, 23 bp, Artificial Sequence
  Synthetic primer
ctctgtaactgcttataatcctg

SEQ ID NO: 19 - DNA, 21 bp, Artificial Sequence
  Synthetic primer
ctaggaaacctgtacaactcc

SEQ ID NO: 20 - DNA, 21 bp, Artificial Sequence
  Synthetic primer
ggcttattgtgtgctgatatc

SEQ ID NO: 21 - DNA, 21 bp, Artificial Sequence
  Synthetic primer
agagatccttaagtcgtcatg

SEQ ID NO: 22 - DNA, 22 bp, Artificial Sequence
  Synthetic primer
catgacgacttaaggatctctt

SEQ ID NO: 23 - DNA, 21 bp, Artificial Sequence
  Synthetic primer
ctcagtttccagagtacaaac

SEQ ID NO: 24 - DNA, 21 bp, Artificial Sequence
  Synthetic primer
cagtttctgtgagagagtaca

SEQ ID NO: 25 - DNA, 22 bp, Artificial Sequence
  Synthetic primer
ggcttacctgctcctggtattt

SEQ ID NO: 26 - DNA, 22 bp, Artificial Sequence
  Synthetic primer
gtgaattaaagtctttctggcc

SEQ ID NO: 27 - DNA, 21 bp, Artificial Sequence
  Synthetic primer
atcttagaaagcagacagggc

SEQ ID NO: 28 - DNA, 21 bp, Artificial Sequence
  Synthetic primer
gagacattttatccccttgtg

SEQ ID NO: 29 - DNA, 21 bp, Artificial Sequence
  Synthetic primer
tccatgcctccagtctaaagt

SEQ ID NO: 30 - DNA, 21 bp, Artificial Sequence
  Synthetic primer
gaggaggaatgggcctttatt

SEQ ID NO: 31 - DNA, 20 bp, Artificial Sequence
  Synthetic primer
acccacagaatagggcagga

SEQ ID NO: 32 - DNA, 20 bp, Artificial Sequence
  Synthetic primer
cacttaagttgcactgggta

SEQ ID NO: 33 - DNA, 21 bp, Artificial Sequence
  Synthetic primer
caacaggaagttggtctcatc

SEQ ID NO: 34 - DNA, 21 bp, Artificial Sequence
  Synthetic primer
taaaaggaagagcggctgttt

SEQ ID NO: 35 - DNA, 21 bp, Artificial Sequence
  Synthetic primer
ttaaacctaactgccaccctc

SEQ ID NO: 36 - DNA, 22 bp, Artificial Sequence
  Synthetic primer
ggatactggcattctgtgtaac

SEQ ID NO: 37 - DNA, 21 bp, Artificial Sequence
  Synthetic primer
atttccagatagtaagcccca

SEQ ID NO: 38 - DNA, 21 bp, Artificial Sequence
  Synthetic primer
agcttggacggaagtcagatc

SEQ ID NO: 39 - DNA, 21 bp, Artificial Sequence
  Synthetic primer
tctagccaaacctcgggtaac

SEQ ID NO: 40 - DNA, 18 bp, Artificial Sequence
  Synthetic primer
aattgtaaacctctgccc

SEQ ID NO: 41 - DNA, 18 bp, Artificial Sequence
  Synthetic primer
atttcccaagctcatgct

SEQ ID NO: 42 - DNA, 18 bp, Artificial Sequence
  Synthetic primer
agcatgagcttgggaaat

SEQ ID NO: 43 - DNA, 18 bp, Artificial Sequence
  Synthetic primer
tgaagacctatctttgcc

SEQ ID NO: 44 - DNA, 21 bp, Artificial Sequence
  Synthetic primer
gttcacagagctcctcacact

SEQ ID NO: 45 - DNA, 21 bp, Artificial Sequence
  Synthetic primer
aggccacagagtcaactatgg

SEQ ID NO: 46 - DNA, 21 bp, Artificial Sequence
  Synthetic primer
aggtcctatcaccaagggtgt

SEQ ID NO: 47 - DNA, 21 bp, Artificial Sequence
  Synthetic primer
gcttagttacttcttcaaggc

SEQ ID NO: 48 - DNA, 22 bp, Artificial Sequence
  Synthetic primer
gagaaagttaatatcctagctg

SEQ ID NO: 49 - DNA, 23 bp, Artificial Sequence
  Synthetic primer
gtgaatggtccatatataaatca

SEQ ID NO: 50 - DNA, 21 bp, Artificial Sequence
  Synthetic primer
tggtttagcacacctcttcac

SEQ ID NO: 51 - DNA, 21 bp, Artificial Sequence
  Synthetic primer
gcttagcacaaaccctgtttc

SEQ ID NO: 52 - DNA, 20 bp, Artificial Sequence
  Synthetic primer
ctgagctatgtgcatttgca

SEQ ID NO: 53 - DNA, 20 bp, Artificial Sequence
  Synthetic primer
aaggctgctgctaaacagat

SEQ ID NO: 54 - DNA, 30 bp, Artificial Sequence
  Synthetic primer
aacacaatacaaacaacaaacaaacaaaca

SEQ ID NO: 55 - DNA, 30 bp, Artificial Sequence
  Synthetic primer
gttgttttaggttttggtttgtgttgtttt

SEQ ID NO: 56 - DNA, 25 bp, Artificial Sequence
  Synthetic primer
gatacgaacgacgaacgaacgaacg

SEQ ID NO: 57 - DNA, 26 bp, Artificial Sequence
  Synthetic primer
ttttaggtttcggttcgcgtcgtttc

SEQ ID NO: 58 - DNA, 29 bp, Artificial Sequence
  Synthetic primer
cctcaacataaacaaaacaacacaaacca

SEQ ID NO: 59 - DNA, 28 bp, Artificial Sequence
  Synthetic primer
tttgagagttttttgaggtgtgtgatgt

SEQ ID NO: 60 - DNA, 26 bp, Artificial Sequence
  Synthetic primer
caacataaacgaaacgacgcgaaccg

SEQ ID NO: 61 - DNA, 23 bp, Artificial Sequence
  Synthetic primer
gagtttttcgaggcgcgcgacgc

SEQ ID NO: 62 - DNA, 29 bp, Artificial Sequence
  Synthetic primer
gaattggtgttttttgagttgtgttgtgt

SEQ ID NO: 63 - DNA, 31 bp, Artificial Sequence
  Synthetic primer
aaaaaccacaaaaaaacacaacaaaaacaaa

SEQ ID NO: 64 - DNA, 24 bp, Artificial Sequence
  Synthetic primer
ggtgtttttcgagtcgcgtcgcgc

SEQ ID NO: 65 - DNA, 26 bp, Artificial Sequence
  Synthetic primer
ccgcaaaaaaacgcgacgaaaacgaa

SEQ ID NO: 66 - DNA, 21 bp, Artificial Sequence
  Synthetic primer
gccttccgagccgcgtcgcgc

SEQ ID NO: 67 - DNA, 22 bp, Artificial Sequence
  Synthetic primer
aggaaagcgcggcgagagcggg

SEQ ID NO: 68 - DNA, 20 bp, Artificial Sequence
  PAI-2 promoter forward primer
tgacaaactcaacaagtgga

SEQ ID NO: 69 - DNA, 20 bp, Artificial Sequence
  PAI-2 promoter reverse primer
tgcataagataaccaactgc

SEQ ID NO: 70 - DNA, 26 bp, Artificial Sequence
  beta-actin forward primer
tgacgggctcacccacactgtgccca

SEQ ID NO: 71 - DNA, 26 bp, Artificial Sequence
  beta-actin reverse primer
ctagaagcatttgcggtggacgatgg

SEQ ID NO: 72 - DNA, 20 bp, Artificial Sequence
  PAI-2 promoter forward primer
atacccgaagaaaattagga

SEQ ID NO: 73 - DNA, 21 bp, Artificial Sequence
  PAI-2 promoter reverse primer
aagttgcagttctaacgtaga

(19) United States
(12) Patent Application Publication
(10) Pub. No.: US 2009/0270479 A1
(43) Pub. Date: Oct. 29, 2009

(54) Genetic and Epigenetic Alterations In the Diagnosis and Treatment of Cancer

(76) Inventor: Antonio Giordano, Radnor, PA (US)

Correspondence Address: William J McNichol Jr;Reed Smith, Intellectual Property, P O Box 7990, Philadelphia, PA, 19101-7990, US

(21) Appl. No.: 11/990,031

(22) PCT Filed: Dec. 20, 2005
(86) PCT No.: PCT/US2005/046069

§ 371 Date: Jul. 21, 2008

Related U.S. Application Data
(60) Provisional application No. 60/698,721, filed on Jul. 12, 2005.

(51) Int. Cl. A61K 31/7088; C12Q 1/68; C07H 21/04; C07K 16/00; A61P 35/00

(52) U.S. Cl. 514/44 A; 435/6; 536/23.5; 530/387.9

(57) ABSTRACT

Methylation of DNA in regions involved in transcriptional regulation can induce the binding of ICBP90 and the subsequent formation of multiprotein complexes which alter gene transcription. DNA methylation in tumor suppressor genes, or in other genes which are involved in mitigating tumorigenesis, can induce binding of ICBP90 to those genes. Bound ICBP90 can interact with a pRb2/p130 regulatory complexes to remodel chromatin and inhibit transcription of the gene. DNA methyltransferases, ICBP90, and the proteins comprising the pRb2/p130 complex are therefore therapeutic targets for the treatment of cancer. Abnormalities in these proteins can also be markers of cancerous or precancerous conditions.

### FIELD OF THE INVENTION

This invention relates to methods of diagnosing and treating cancer, and to methods of inhibiting the growth of cancer cells. In particular, the methods of the invention involve inhibiting ICBP90 protein or protein in or associated with pRb2/p130 complexes, detecting mutations in the RB2/p130 gene, or determining the methylation state of the RB2/p130 and other genes.

### BACKGROUND

Retinoblastoma is the most common intraocular malignancy in children. Human retinoblastoma occurs in two forms: a nonheritable form, which is usually unilateral, and a heritable form, which is often bilateral with autosomal dominant expression. Both forms have been ascribed to biallelic mutation of the Rb1/p105 gene and the consequent loss of its tumor-suppressive functions. The basic function of pRb1/p105 is to hold cells in G1 or G0 phase of the cell cycle and prevent entry into S phase by interacting and negatively regulating the E2F family of transcription factors. Moreover, pRb1/p105 is also involved in the apoptotic response by interacting with p53 pro-apoptotic pathway. Notwithstanding the fact that mutation of pRb1/p105 is common to all retinoblastomas, much evidence indicates that loss of pRb1/p105 from a developing retinal cell is insufficient for malignancy (DiCiommo et al., 2000, Semin. Cancer Biol., 10, 255-269).

pRb1/p105 functions are shared by two homologous proteins, pRb2/p130 and p107, so that the three of them are referred to as retinoblastoma family proteins (pRBs). In particular, RB2/p130 gene has been found mutated or functionally inactivated in many tumors, and its role in controlling p53-independent apoptotic response has been elucidated recently (La Sala et al., 2003, Oncogene, 22, 35183529.). Expression of pRb2/p130 is impaired in some sporadic retinoblastomas, and loss of expression correlates with low apoptotic index. However, while genetic alterations in RB2/p130 gene have been reported, to date there are insufficient data to link the loss of pRb2/p130 expression with the mutational status of this gene. For example, studies have reported mutations of RB2/p130 in non-small cell lung cancer and small cell lung cancer, respectively, but these mutations only partially justify the absence of the protein (Helin et al., 1997, P.N.A.S USA 94: 6933-6938; Claudio et al., 2000, Cancer Res 60: 372-382).

Although the importance of genetic alterations in cancer has been long recognized, the role of epigenetic changes in affecting tumor formation and progression has been suggested only recently. Epigenetic events are mediated by DNA methylation and chromatin remodeling via histone acetylation, methylation and phosphorylation, which lead to the formation of transcriptionally repressive chromatin states resulting in gene silencing. Accumulating evidence indicates that CpG island hypermethylation in the promoter regions of regulatory genes is an early event in cancer development and may precede the neoplastic process.

Different studies have been suggested that aberrant methylation may inactivate cancer-related genes in lung tumors, but the role of RB2/p130 tumor suppressor gene in lung carcinogenesis is not yet well defined. Although several reports have demonstrated that about 30% of lung tumors exhibit absence or reduced expression of pRb2/p130 protein, there are conflicting data regarding the genetic and epigenetic events responsible of the aberrant expression of this gene in cancers (Claudio et al, supra; Modi et al., 2000, Oncogene 19: 4632-4639; Xue et al., 2003, Mol. Carcinog. 38: 124-129).

The pRb2/p130 protein can interact with other proteins to form multi-protein complexes, which can affect the transcription of certain genes. The recruitment of pRb2/p130 and other proteins into such multi-protein complexes may be directly correlated with a specific transcriptional environment, for example as defined by the methylation state of the DNA.

The “Inverted CCAAT box Binding Protein of 90 kDa” or “ICBP90” is a recently identified nuclear protein that binds to one of the inverted CCAAT boxes in the topoisomerase IIalpha (TopoIIalpha) gene promoter. ICBP90 localizes in cell nuclei and contains an ubiquitin-like (UbL) domain, a leucine zipper, a zinc-finger of the PHD-finger type, an SRA domain, two nuclear localization signals (NLSs) and a zinc-finger of the ring-finger type. ICBP90 mRNA is abundantly expressed in actively proliferating tissues. Similarly, ICBP90 protein is highly expressed in cultured fibroblasts at the active proliferative stage, but not after the cells reached confluence. ICBP90 shares structural homology with several other nuclear proteins, including Np95 and the human and mouse NIRF, suggesting the emergence of a new family of nuclear proteins involved in transcriptional regulation.

Cancer cell lines express higher levels of ICBP90 and TopoIIalpha than non-cancerous cell lines. For example, in primary cultured human lung fibroblasts, ICBP90 expression peaks at late G1 and during G2/M phases. In contrast, HeLa, Jurkat and A549 cancer cell lines show constant ICBP90 expression throughout the entire cell cycle.

The plasminogen activator inhibitor type-2 (PAI-2) is a member of the ovalbumin subgroup of serpins (ov-serpins), originally characterized in human placenta and macrophages. PAI-2 is synthesized by a variety of cells, including tumor cells, after appropriate stimulation. Extracellular PAI-2 is a potent inhibitor of urokinase-type plasminogen activator (u-PA), Different studies have indicated that PAI-2 acts as a multifunctional protein, since it is involved in the regulation of fibrinolysis, the regulation of keratinocytes development, cellular proliferation, the invasion and metastasis of cancer cells, and in conferring resistance to apoptosis. Heretofore, the intracellular targets and regulatory mechanism of PAI-2 expression were largely undefined. However, it has been recently reported that in Hela and in Jurkat cells PAI-2 expression could result in posttranscriptional recovery of the retinoblastoma protein Rb and that PAI-2 could inhibit Rb degradation, suggesting an intriguing intranuclear role of PAI-2 (Darnell et al., 2003, Mol. Cell. Biol. 23(18): 6520-6532).

Although treatment strategies for cancer have gradually evolved throughout the past decades, enucleation and radiotherapy are still the most common retinoblastoma treatments, and salvage of useful vision is possible only in limited cases. Chemotherapy for retinoblastoma and other cancers is also a treatment option. However, the high cost of surgery, radiotherapy and conventional chemotherapy, and the occurrence of debilitating side effects, make such treatments undesirable.

What is needed, therefore, is a better understanding of the genetic and epigenetic events surrounding tumorigenesis in retinoblastoma, lung cancer and other cancers. More effective and economic diagnostic and treatment strategies can then be generated to treat such conditions.

### SUMMARY OF THE INVENTION

Methylation of DNA in regions involved in transcriptional regulation can induce the binding of ICBP90 and the subsequent formation of multiprotein complexes which alter gene transcription. For example, DNA methylation in tumor suppressor genes, or in other genes which are involved in mitigating tumorigenesis, can induce binding of ICBP90 to those genes. Bound ICBP90 interacts with a pRb2/p130 complex to remodel chromatin and inhibit transcription of the gene. DNA methyltransferases, ICBP90, and the proteins comprising the pRb2/p130 complex are therefore therapeutic targets for the treatment of cancer. Abnormalities in these proteins can also be markers of cancerous or precancerous conditions.

For example, biallelic mutations have been discovered in various cancer cell lines and primary tumors coming from different embryonal origin. These missense mutations occur in a region of exon 1 of the RB2/p130 gene which is rich in CpG islands. The methylation state of the regions in and around exon 1 of the RB2p130 gene is altered in cells which have one or both of the exon 1 mutations, and such alterations are correlated with a reduction in RB2/p130 gene expression. Agents which demethylate the RB2/p130 gene restore pRb2/p130 levels in cells. Thus, both genetic and epigenetic events in the RB2/p130 gene down-regulate RB2/p130 gene expression in cancer, and agents which inhibit or reverse these events can be used to treat cancer.

The invention thus provides a method of detecting tumor cells or diagnosing cancer in a subject, comprising the steps of obtaining a biological sample comprising test cells from a subject and obtaining nucleic acid from the test cells. The nucleic acid obtained from the test cells can be analyzed for mutations in exon 1 of the RB2/p130 gene, wherein the presence of homozygous mutations at nucleotides 178 and/or 259 of the RB2/p130 gene indicate that the test cells are tumor cells or that the subject has cancer. Alternatively, DNA obtained from the test cells can be analyzed the methylation status of the RB2/p130 gene, wherein methylation of at least the region from about nucleotide +287 to about +411 of the RB2/p130 gene indicates that the test cells are tumor cells or that the subject has cancer.

The invention also provides a method of detecting cells which are predisposed to tumorigenesis, comprising obtaining a biological sample comprising test cells from a subject, wherein the test cells appear histologically or morphologically normal. Nucleic acid is obtained from the test cells, and can be analyzed for mutations in exon 1 of the RB2/p130 gene. The presence of homozygous mutations at nucleotides 178 and/or 259 of the RB2/p130 gene indicate that the test cells are predisposed to tumorigenesis. Alternatively, DNA obtained from the test cells can be analyzed the methylation status of the RB2/p130 gene, wherein methylation of at least the region from about nucleotide +287 to about +411 of the RB2/p130 gene indicates that the test cells are predisposed to tumorigenesis.

The invention further provides a method of treating cancer, comprising the steps of providing a subject who has, or is at risk for developing, cancer, in which the cells of the subject have a homozygous mutation at nucleotides 178 and/or 259 of the RB2/p130 gene or have methylation of at least the region from about nucleotide +287 to about +411 of the RB2/p130 gene. The cancer is treated by administering an effective amount of a demethylating agent to the subject.

The invention yet further provides a method of inhibiting uncontrolled growth in cells that have a homozygous mutation at nucleotides 178 or 259 of the RB2/p130 gene or have methylation of at least the region from about nucleotide +287 to about +411 of the RB2/p130 gene. The method comprises the step of contacting the cells with an effective amount of a demethylating agent, such that the methylation status of the RB2/p130 gene in the cells is altered.

The invention still further provides nucleic acid sequences comprising a C to T transition at nucleotides 178 and/or a C to G transversion at nucleotide 259 of the RB2/p130 gene.

The invention still further provides nucleic acid primers comprising sequences designed to amplify exons 1 through 22 of the RB2/p130 gene, and to amplify and discriminate methylated from un-methylated regions in exon 1, intron 1, and the promoter region immediately upstream of the transcription start site of the RB2/p130 gene.

The invention still further provides mutant pRb2/p130 proteins, comprising a substitution of serine for proline at codon 37 and/or a substitution of proline for alanine at codon 64 of the pRB2/p130 protein. The invention also provides antibodies specific for the mutant pRb2/p130 proteins.

The invention still further provides a method of detecting tumor cells or diagnosing cancer in a subject, comprising the steps of obtaining test cells from a subject and obtaining protein from the test cells. The protein obtained from the test cells can be analyzed for mutations in pRB2/p130 protein, wherein the presence of a substitution of serine for proline at codon 37 and/or a substitution of proline for alanine at codon 64 of the pRB2/p130 protein indicate that the test cells are tumor cells or that the subject has cancer.

The invention also provides a method of detecting cells which are predisposed to tumorigenesis, comprising obtaining a biological sample comprising test cells from a subject, wherein the test cells appear histologically or morphologically normal. Protein is obtained from the test cells, and can be analyzed for the presence of a substitution of serine for proline at codon 37 and/or a substitution of proline for alanine at codon 64 of the pRB2/p130 protein. The presence of such substitutions at codons 37 and/or 64 in pRb2/p130 indicate that the test cells are predisposed to tumorigenesis.

The invention further provides a method for detecting sporadic retinoblastoma tumor cells or for diagnosing sporadic retinoblastoma in a subject, comprising the steps of obtaining a biological sample comprising test cells from a subject and obtaining nucleic acid from the test cells. The nucleic acid obtained from the test cells can be analyzed for mutations in exon 12 of the RB2/p130 gene, wherein the presence of a homozygous mutation at nucleotide 1650 of the RB2/p130 gene indicate that the test cells are tumor cells or that the subject has sporadic retinoblastoma.

The invention further provides a method of treating cancer or inhibiting proliferation of tumor cells, comprising inhibiting the binding of ICBP90 to regions of DNA involved in transcriptional regulation of a tumor suppressor gene or other gene involved in mitigating tumorigenesis. Inhibiting the binding of ICB90 allows transcription of the tumor suppressor or other gene, by reducing formation of multiprotein complexes which inhibit gene transcription.

The invention still further provides a method of treating cancer or inhibiting proliferation of tumor cells, comprising inhibiting the formation of multi-protein transcriptional repressor complexes on a tumor suppressor gene or other gene involved in mitigating tumorigenesis, thus allowing transcription of the tumor suppressor or other gene.

### BRIEF DESCRIPTION OF THE DRAWINGS

FIG. 1 is a schematic showing that tumorigenesis may spring from the combined forces of both genetic and epigenetic events in the RB2/p130 gene. Mutations at nucleotide 178 (TCT→CCT) and 259 (CCC→GCC) of RB2/p130 exon 1 determine the substitution of serine to proline (codon 37) and proline to alanine (codon 64) of pRb2/p130, respectively. FIG. 1a) The amino acid substitutions resulting from the RB2/p130 exon 1 mutations may lead to protein conformation changes impairing the pRb2/p130 stability and function. The RB2/p130 exon 1 mutations could also be the “hit event” that predisposes the RB2/p130 gene to epigenetic changes leading to inhibition of RB2/p130 gene expression, resulting in tumorigenesis.

FIG. 2a is a schematic representation of Region 1, Region 2 and Region 3 CpG methylation sites on the RB2/p130 promoter, exon 1 and intron 1.

FIG. 2b shows agarose gel electrophoreses of nucleic acid fragments amplified from Region 1, Region 2 and Region 3 of the RB2/p130 gene by methylation specific PCR (“MSP”), for three representative samples. MSP amplification was performed with specific primers for methylated (M1, M2 and M3) and unmethylated (U1, U2 and U3) modified DNA. Retinoblastoma unmodified (C1, positive control) and modified (C2, negative control) DNA were amplified with wild-type primers. The sample with normal RB2/p130 expression (+++) shows Region 1, Region 2 and Region 3 as being unmethylated (U1, U2 and U3); the sample with weak RB2/p130 expression (+) shows only Region 3 (3) as being methylated. The sample with negative RB2/p130 expression (−) shows all Region 1, Region 2 and Region 3 as being methylated (M1, M2 and M3).

FIGS. 3a-3g show pRb1/p105 and pRb2/p130 protein expression levels and mutational analysis in human cell lines and in tumor samples. FIG. 3a is a Western Blot analysis of pRb1/p105 and pRb2/p130 in Jurkat cell line used as positive control for antibodies (C), normal retina (NR), Weri-Rb1 cell line (W) and in frozen retinoblastoma samples (8-10). Anti-actin antibody was been used as loading control. FIGS. 3b and 3c are immunohistochemical analyses of pRb2/p130 in normal retina (b) and in a representative retinoblastoma sample (c). FIGS. 3d-3f represent the laser capture microdissection of two selected areas (e, f) of paraffin-fixed tumor sample (d). FIG. 3g shows exon 1 homozygous missense mutations at RB2/p130 codons 178 and 259 detected in a representative DNA sample obtained by laser capture microdissection. The sequences were matched with RB2/p130 wild-type sequences.

FIGS. 4a-4b show the effect of 5-Aza-dC treatment on Weri-Rb1 cells. FIG. 4a is a proliferation analysis of Weri-Rb1 cells treated with 2.5 μm 5-Aza-2-dc at different times (24, 48 and 96 h). The proliferation index was calculated as a percentage of the signal of sample relative to the signal of untreated cells at 0 h. FIG. 4b is a Western blot analysis of pRb2/p130 using whole cell lysates from Weri-Rb1 cell line treated with 2.5 mM 5-Aza-2-dc at different times (24, 36, 48, 72 and 96 h). Whole cell lysates from untreated Weri-Rb1 cells harvested at 96 h were used as control (C). Equal concentration of 100 mg of total proteins has loaded in each well (Actin).

FIGS. 5a-5d how RB2/p130 mRNA and pRb2/p130 protein expression levels and mutational analysis in human lung adenocarcinoma (H23) cell line. FIG. 5a is a Northern Blot analysis of RB2/p130 mRNA in H23 and control (Saos-2) cells. Loading and integrity of RNA was confirmed by hybridization with a glyceradehyde-3-phosphate dehydrogenase (GAPDH) cDNA probe. FIG. 5b is a Western Blot analysis of pRb2/p130 in H23 and control (Saos-2) cells; β-lactin was used to normalize. FIGS. 5c-5d are mutational analyses performed by amplifying and sequencing exon 1 of RB2/p130 in H23 cells. The sequences were matched with the Wild-type sequences. Nucleotide changes have been found at codon 37 and 64.

FIG. 6 shows an analysis of RB2/p130 methylation status by methylation-specific PCR RB2/p130 promoter in H23 cells. Region 1 (M1) and region 3 (M3) are methylated while region 2 is unmethylated (U2). H23 unmodified DNA amplified with wild-type primers (C1). H23 DNA modified amplified with wild-type primers (C2).

FIG. 7 shows the effect of demethylating agent 5-AZA-2-deoxycytidine on RB2/p130 expression at different treatment times. FIG. 7a is an agarose gel electrophoresis of multiplex RT-PCR using total RNA from H23 cells untreated (C) and treated with 2.5 μM 5-AZa-2-deoxycytidine at different times (24, 36, 48, 72 and 96 hours). RB2/p130 mRNA level increased respect to basal level (C) after the treatment beginning at 48 hours; β-actin was used to normalize protein loading levels. FIG. 7b is a Western blot analysis using whole cell lysates from H23 cells treated with 2.5 μM 5-AZA-2-deoxycytitdine at different times (24, 36, 48, 72 and 96 hours). The level of pRb2/p130-active hyposphorylated form increased beginning at 72 hours from the treatment and reached a maximum at 96 hours; β-actin was used to normalize protein loading levels. The upper band represents the phosphorylated form of pRb2/p130 (pRb2/p130-P) and the lower band represents the pRb2/p130 unphosphorylated form (pRb2/p130).

FIG. 8 is a Western blot analysis showing protein expression levels of ICBP90 in whole cell lysates (“tot lys”) of MCF-7, MDA-MB-231 and MDA-MB-361 cultured breast cancer cells. β-actin was used to normalize protein loading levels.

FIG. 9 is an immunoprecipitation (“IP”) analysis showing ICBP90 protein levels in the nuclear and cytoplasmic fractions of MCF-7, MDA-MB-231 and MDA-MB-361 cultured breast cancer cells. Efficient cytoplasmic and nuclear fractionation was confirmed by immunoblot analysis using anti-glyceraldehyde-3-phosphate dehydrogenase (GAPDH) antibody for the cytoplasmic fraction, and anti Oct-1 antibody for the nuclear fraction.

FIG. 10a is a schematic showing regions 1 (−722 to −458) and 2 (−255 to −130) of the estrogen receptor α promoter. The location of the CAAT and TATA boxes and the binding sites for E2F transcription factors are indicated. The transcriptional start site is indicated by the curved, solid black arrow. PCR primers flanking estrogen receptor α regions 1 (“WTNF” and “WTNR”) and 2 (“WT1” and “WT2”) are also indicated. FIG. 10b is a cross-linked chromatin immunoprecipitation (“XChIP”) analysis of ICBP90 binding to estrogen receptor α promoter regions 1 and 2, showing that ICBP90 binds to estrogen receptor α promoter region 1 (ER-α Reg1) but not region 2 (ER-α Reg2). DNA sequences were amplified by PCR using the forward and reverse primers flanking the estrogen receptor α promoter regions as indicated in FIG. 10a. “IP:ICBP90” indicates amplification of an immunoprecipitation reaction of ICBP90 and immunoprecipitated chromatin. One percent of total chromatin (“inputs”) was used as a positive control in PCR reactions, and no-antibody immunoprecipitations were performed as a negative control in PCR reactions (not shown).

FIG. 11 is a schematic showing the proposed mechanism of transcriptional repression mediated by DNA methylation and ICBP90. The open circles are unmethylated CpG, and the closed circles are methylated CpG. “HDAC1” is histone deacetylase 1; “SUV39H1” is histone methyl transferase; “DNMT1” is DNA methyl transferase 1; “p300” is histone acetyl transferase; “RNA Pol II” is RNA polymerase II; “E2 F4/5” is E2F transcription factor 4/5; “TBP” is TATA binding protein; “TAFs” are TATA-associated factors; “TF2” is Transcription Factor 2.

FIG. 12 shows an immunoprecipitation of PAI-2 from nuclear (“IP PAI-2 nuclear”) and cytoplasmic (“IP PAI-2”) fractions from three representative cases of paired primary cornea (“Corn”) and conjunctiva (Conj) cells by using an anti-PAI-2 antibody, followed by electrophoresis and Western blotting of the immunoprecipitates with anti-pRb2/p130, anti-Rb1/p105, anti-p107 and anti-PAI-2 antibodies. Control (lane 1) represents Western blotting of nuclear or cytoplasmic immunoprecipitates where the anti-PAI-2 antibody was omitted. “Corn1” and “Conj1”: donor 1; “Corn2” and “Conj2”: donor 2; “Corn3” and “Conj3”: donor 3. FIG. 12b is a Western blot analysis of equal amounts of total lysates from cornea (“Corn Tot Lysate”) and conjunctiva (“Conj Tot Lysate”) cells, with anti-pRb2/p130, anti-Rb1/p105, anti-p107 and anti-PAI-2 antibodies. FIG. 12c is an immunoblot analysis showing the purity of the nuclear and cytoplasmic fractions in FIG. 12a, with anti-GAPDH, as cytoplasmic marker and anti-Oct1, as nuclear marker.

FIG. 13a is a schematic representation of region 1 of the PAI-2 promoter recognized by P1/P2 primers (GenBank accession no. M22469). FIG. 13b shows representative results from XChIP analyses in human primary corneal and conjunctival cells. Formaldehyde cross-linked chromatin was immunoprecipitated using the antibodies indicated on the top of each panel. The presence of PAI-2 promoter region in the immunoprecipitates was tested by PCR using specific primers (P1/P2) spanning the region 1 of PAI-2 promoter. 1% of total chromatin (inputs) was used as a positive control in PCR reactions. No-antibody immunoprecipitations were performed as a negative control in PCR reactions (not shown).

FIG. 14a is an agarose gel electrophoresis showing the steady-state of PAI-2 mRNA levels in four representative cases of paired primary cornea (“Corn”) and conjunctiva (“Conj”) normal cells. Multiplex RT-PCR was performed using total cellular RNA from Corn and Conj cells. Each RT-PCR reaction contained 1/100 of cDNA. 0.3:2.0 was the primer ratio for β-actin and PAI-2 used to amplify both products logarithmically and in relatively similar amounts. The upper band and lower band in each line represent PCR products of β-actin and PAI-2-gene, respectively. The panels showing the results from the multiplex RT-PCR, are representative of 4 separate experiments. FIG. 14b is a histogram showing the relative PAI-2 expression levels in Corn and Conj cells. The values were calculated as the density of the product of PAI-2 gene divided by that of the β-actin from the same cDNA.

FIGS. 15a and 15b are schematics showing pRb2/p130 co-repressor complexes on the PAI-2 gene in corneal and conjunctival cells, respectively. “HDAC1” is histone deacetylase 1; “SUV39H1” is histone methyl transferase; “DNMT1” is DNA methyl transferase 1; “E2 F5” is E2F transcription factor 5; “TATA” are TATA-associated factors; “PAI-2” is the inhibitor of urokinase-type plasminogen activator (the product of the PAI-2 gene).

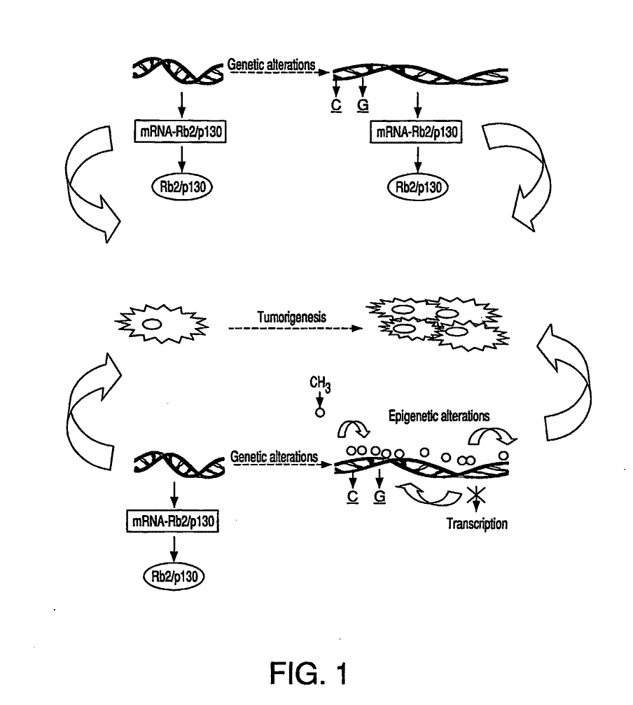

### DETAILED DESCRIPTION OF THE INVENTION

As generally used herein, a gene or mRNA appears in italics, and the protein produced by the gene or RNA appears in regular test. For example, the retinoblastoma tumor suppressor gene (pRb) is designated as “RB2/p130,” or “RB2/p130 gene” and the RNA is designated as “RB2/p130 RNA,” and the protein produced from the pRB2/p130 gene or RNA is designated as “pRb2/p130” or “pRb2/p130 protein.”

Epigenetic events, such as DNA methylation, are believed to play a role in gene transcription. The methylation status of a given gene can dictate whether proteins from the retinoblastoma gene family interact with transcription factors such as the E2Fs and chromatin-modifying enzymes to inhibit or enhance transcription of that gene. The exact composition of these multi-protein transcriptional regulation complexes may differ from gene to gene. However, without wishing to be bound by any theory, certain proteins such as pRb2/p130 and ICBP90 appear to have a central role in the initiation and/or maintenance of multi-protein transcriptional regulation complexes.

ICBP90 binds with high affinity to methylated CpGs (i.e., a cytosine followed by a guanosine in the 3′-direction, or 5′-CG-3′) in DNA through its SRA domain, and induces HDAC1 to bind to DNA through the same SRA domain. ICBP90, whose expression is directly regulated by E2F-1, thus targets methylated promoter regions of various tumor suppressors. HDAC1 is part of the pRb2/p130 co-repressor complex which inhibits expression of the gene to which it is bound. Again without wishing to be bound by any theory, the following model can be envisioned for transcriptional repression of tumor suppressor genes or other genes involved in mitigating tumorigenesis.

DNA in a gene promoter or other regulatory region is methylated by endogenous DNA methylation enzymes, for example DNMT1, DNMT3a and DNMT3b. ICBP90 binds to methylated CpGs, and HDAC1, pRb2/p130 protein and other proteins in the pRb2/p130 co-repressor complex (such as E2F transcription factors) assemble on the DNA. It is understood that the order in which the proteins comprising the pRb2/p130 regulatory complex are discussed is not necessarily the order in which they are recruited to the complex. For example, pRb2/p130 or HDAC1 or other proteins can bind first to ICBP90. It is also understood that the constituents of the pRb2/p130 complex can vary, although the presence of at least pRb2/p130, HDAC1, E2F transcription factors (such as E2F 4/5) are considered important. In addition to HDAC1, other chromatin remodeling enzymes (such as histone methyl transferase SUV39H1 and histone acetyl transferase p300), can be present in the complex. The assembly of the pRb2/p130 complex leads to remodeling of the local chromatin structure and inhibition of gene transcription. It is understood that formation of the pRb2/p130 regulatory complex can enhance (“co-stimulatory”) or suppress (“co-regulatory complex”) expression of a given gene.

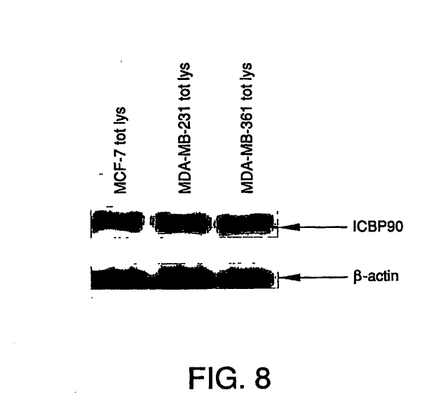

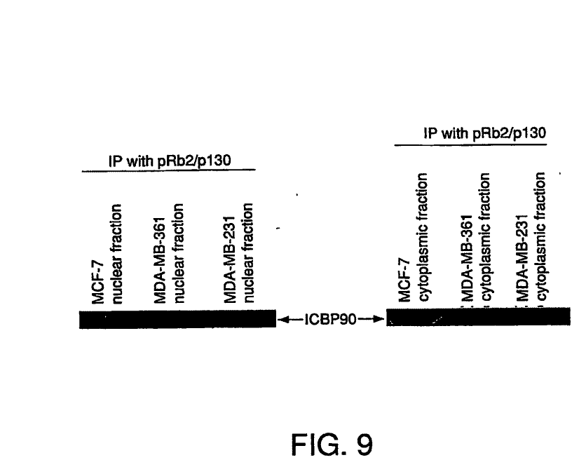

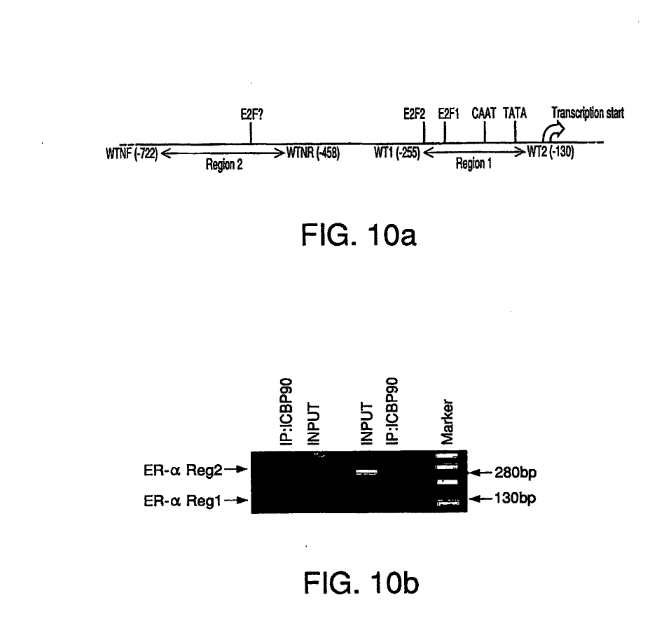

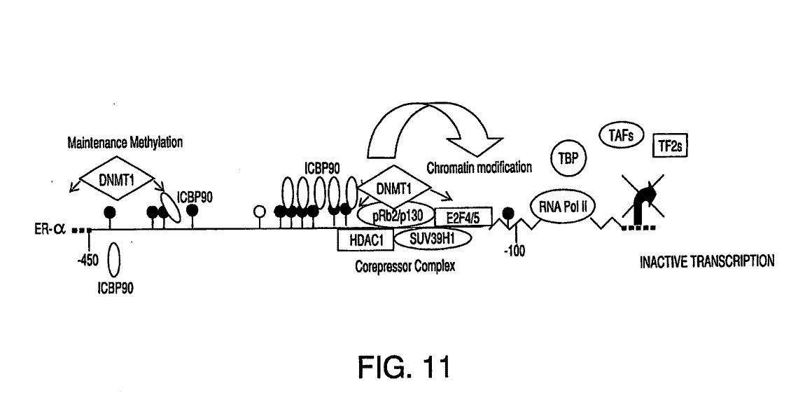

The inhibition of estrogen receptor (“ER”)-α gene expression by a pRb2/p130 co-repressor complex is illustrative of the general transcriptional inhibition process initiated by ICBP90 binding to methylated promoter regions. ICBP90 is expressed in breast cancer cells (see FIG. 8), and is generally found in the nucleus (see FIG. 9). Immunoprecipitation studies show that ICBP90 is associated with region 1, but not region 2, of the ER-α gene promoter (see FIGS. 10a and 10b). The interaction between the pRb2/p130-complex and ICBP90 leads to the recruitment of DNMT1, and concomitant release of histone acetyl transferase p300 from the complex. The pRb2/p130 complex represses the ER-α transcription by maintaining a closed chromatin conformation that does not allow assembly and/or escape of the RNA polymerase II (“RNA Pol II) complex. This interaction of ICBP90 and the pRb2/p130 complex is shown schematically in FIG. 11. DNA methylation, histone deacetylation and methylation, and perhaps ubiquitination of H3 in this context could set up a heritable “mark” and establish a state of long-term silencing in the heterochromatin.

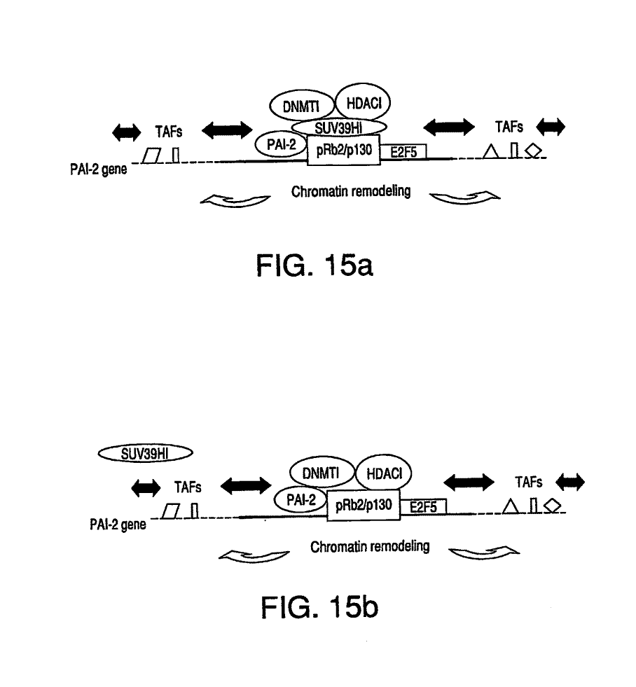

Another illustrative example of an ICBP90-induced regulation of PAI-2 gene is the transcriptional regulation of the PAI-2 gene by a pRb2/p130 complex comprising the PAI-2 protein. As shown in the Examples below, pRb2/p130 and pRb1/p105, but not p107, interact with PAI-2 in both the cytoplasm and nucleus of normal primary human corneal and conjunctival epithelial cells. Moreover, a specific fragment of the PAI-2 promoter is bound simultaneously by pRb2/p130, PAI-2, E2F5, HDAC1, DNMT1, and SUV39H1 in normal primary human corneal epithelial cells, and by pRb2/p130, PAI-2, E2F5, HDAC1, and DNMT1 in normal primary human conjunctiva epithelial cells. Without wishing to be bound by any theory, in the corneal and conjunctival epithelia it appears that the distribution cytoplasm/nucleus of PAI-2 can be controlled by the interaction with pRb2/p130 and Rb1/p105 through multiple pathways. For instance, under specific stimuli and/or at specific times of cell cycle, pRb2/p130 and Rb1/p105 could shuttle PAI-2 between cytoplasm and nucleus, thus controlling the concentration of PAI-2 in these cellular compartments. In addition, PAI-2 may preserve pRb2/p130 and Rb1/p105 from a rapid degradation. The interaction of the components of the pRb2/p130 complexes are shown in FIGS. 15a and 15b. Although the exact constituents of the complexes vary between corneal and conjunctival cells, binding of the complexes on a specific region of the PAI-2 promoter may modulate the PAI-2 basal transcription by inducing local changes in chromatin structure, which alters the activity of transcription regulators bound nearby.

The ICBP90 pRb2 complex transcriptional regulation model provides several therapeutic targets for the treatment of cancer. For example, the proteins which are part of, or which initiate or are associated with, pRb2p130 complexes (e.g., pRb2/p130, ICBP90, PAI-2, p300, SUV39H1, HDAC1, E2F and other transcription factors, certain DNA methyltransferases) can be inhibited or inactivated. Such inhibition or inactivation can take place directly or indirectly. For example, direct inhibition of proteins can occur by biding antibodies or other proteins, aptamers, or other molecules which block specific sites or otherwise prevent the interaction of a pRb2p130 complex protein with DNA and/or other pRb2p130 complex or other proteins. For example, DNA methyltransferases (e.g., DNMT1, DNMT3a, DNMT3b) can be inhibited with substances, described in more detail below, which prevent the transfer of methyl groups to cytosines in DNA. Indirect inhibition of a pRb2p130 complex can occur by preventing transcription or translation of the mRNA which codes for the protein.

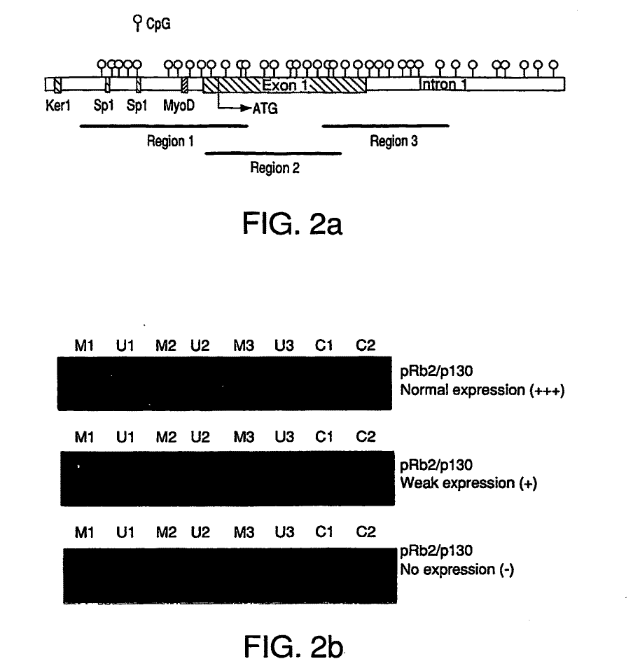

For example, transcriptional inhibition of the RB2/p130 gene is believed to be linked to development and progression of retinoblastoma, lung cancer and other cancers. Without wishing to be bound by any theory, it is believed that transcriptional repression of the RB2/p130 gene is mediated by the methylation state of regions of the RB2/p130 gene in and around exon 1. In particular, methylation of at least the region of the RB2/p130 gene from about nucleotide +287 to about nucleotide +411 the RB2/p130 gene inhibits transcription of this gene (see FIG. 2a).

As is demonstrated in the Examples below, the methylation state of RB2/p130 is affected by mutations in exon 1 of that gene. Two novel RB2/p130 exon 1 mutations have been identified, which can occur separately or together. The first is a cytosine (C) to thymine (T) transition at nucleotide 178 of RB2/p130 exon 1, and the second is a C to guanine (G) transversion at nucleotide 259 of RB2/p130 exon 1. The cDNA sequence of human wild-type RB2/p130 is given in SEQ ID NO: 1. An isolated cDNA sequence of RB2/p130 having the C to T transition at nucleotide 178 is given in SEQ ID NO: 3, and an isolated cDNA sequence of RB2/p130 having the C to G transversion at nucleotide 259 is given in SEQ ID NO: 5. An isolated cDNA sequence of RB2/p130 having the C to T transition at nucleotide 178 and the C to G transversion at nucleotide 259 is given in SEQ ID NO: 7. Unless otherwise indicated, the numbering of nucleotides in the RB2/p130 sequences disclosed herein is relative to SEQ ID NO: 1, with the first nucleotide of SEQ ID NO: 1 being “nucleotide 1.” Thus, “nucleotide 178” or “nucleotide 259” refers to the 178^{th} or 259^{th} nucleotide of SEQ ID NO: 1, respectively.

The invention thus provides isolated nucleic acid sequences comprising the RB2/p130 exon 1 mutations described herein. In one embodiment, the isolated nucleic acids of the invention comprise a nucleic acid sequence which encodes a mutant pRb2/p130 protein of the invention; e.g., a plasmid or viral expression vector comprising SEQ ID NOS: 3, 5 or 7.

Any plasmid vector capable of accepting the nucleic acid coding sequences for can be used in the present invention, for example pBR322, the pUC vectors, pMB1, and vectors derived directly or indirectly from these. Suitable plasmid vectors can be obtained from the American Type Culture Collection (ATCC; Manassas, Va.). Selection of plasmids suitable for expressing isolated nucleic acid sequences of the invention, methods for inserting such nucleic acid sequences into the plasmid are within the skill in the art; see, for example Tuschl, T. (2002), Nat. Biotechnol, 20: 446-448; Brummelkamp T R et al. (2002), Science 296: 550-553; Miyagishi M et al. (2002), Nat. Biotechnol. 20: 497-500; Paddison P J et al. (2002), Genes Dev. 16: 948-958; Lee N S et al. (2002), Nat. Biotechnol. 20: 500-505; and Paul C P et al. (2002), Nat. Biotechnol. 20: 505-508, the entire disclosures of which are herein incorporated by reference.

Any viral vector capable of accepting the coding can be used in the present invention, for example vectors derived from adenovirus (AV); adeno-associated virus (AAV); retroviruses (e.g., lentiviruses (LV), Rhabdoviruses, murine leukemia virus); herpes virus, and the like. Selection of viral vectors suitable for use in the invention, methods for inserting nucleic acid into such vectors are within the skill in the art; see, for example, Dornburg R (1905), Gene Therap. 2: 301-310; Eglitis M A (1988), Biotechniques 6: 608-614; Miller A D (1990), Hum Gene Therap. 1: 5-14; Anderson W F (1998), Nature 392: 25-30; and Rubinson D A et al., Nat. Genet. 33: 401-406, the entire disclosures of which are herein incorporated by reference.

The RB2/p130 exon 1 mutations result in amino acid substitutions in the pRb2/p130 protein. Specifically, the C to T transition at nucleotide 178 results in a substitution of serine for proline at pRb2/p130 codon 37, and the C to G trasversion at nucleotide 259 results in the substitution of proline for alanine at pRb2/p130 codon 64. Thus, the invention provides isolated mutant pRb2/p130 proteins, which comprise either a Ser→Pro substitution at codon 37 (SEQ ID NO: 4) or a Pro→Ala substitution at codon 64 (SEQ ID NO: 6), or both a codon 37 Ser→Pro and a codon 64 Pro→Ala substitution (SEQ ID NO: 8). The invention also provides isolated nucleic acid molecules encoding the mutant pRb2/p130 proteins of the invention; for example, nucleic acid comprising SEQ ID NOS: 3, 5 or 7 in a viral or plasmid expression vector.

The invention also provides isolated antibodies specific for the mutant pRb2/p130 proteins of SEQ ID NOS: 4, 6 and 8. “Antibody” or “antibodies” as used herein include both polyclonal and monoclonal antibodies as well as fragments thereof, such as Fv, Fab and F(ab)₂ fragments that are capable of binding antigen or hapten. Various procedures known in the art can be used for producing isolated polyclonal antibodies that bind to the mutant pRb2/p130 proteins of the invention or a fragment thereof, but that do not bind to the wild-type pRb2/p130 protein. For example, various host animals can be immunized by injection with the mutant pRb2/p130 proteins or a fragment thereof, including but not limited to rabbits, mice, rats, etc. Various adjuvants can be used to increase the immunological response, depending on the host species, including Freund's (complete and incomplete) adjuvant, mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of isolated monoclonal antibodies that that bind to the mutant pRb2/p130 proteins of the invention or a fragment thereof, but that do not bind to the wild-type pRb2/p130 protein, any technique that provides for the production of antibody molecules by continuous cell lines in culture can be used. Examples of such techniques include the hybridoma and trioma techniques, the human B-cell hybridoma technique, and the EBV-hybridoma technique to produce human monoclonal antibodies.

In the production of isolated monoclonal or polyclonal antibodies according to the invention, screening for the desired antibody can be accomplished by techniques known in the art, such as enzyme-linked immunosorbent assay (ELISA). See below for additional description regarding production of antibodies specific for pRb2/p130 complex and other proteins.

As used herein, an “isolated” molecule is a molecule which is synthetic, or which is altered or removed from the natural state through human intervention. For example, an nucleic acid or protein which is naturally present in a living animal is not “isolated,” but a synthetic nucleic acid or protein which is partially or completely separated from the coexisting materials of its natural state, is “isolated.” An isolated molecule can exist in substantially purified form, or can exist in a non-native environment such as, for example, a cell into which the molecule has been introduced. It is understood that a nucleic acid which has been introduced into a host cell, and which has integrated into that host cell's genome, is considered “isolated” for purposes of this invention in both the host cell and any daughter cells produced from the host cell. Molecules which are produced inside a cell by natural processes, but which are produced from or under the direction of an “isolated” molecule, are also considered to be “isolated” molecules. For example, an isolated nucleic acid can be introduced into a target cell, where it expressed to produce RNA or protein The RNA or protein molecules produced from the nucleic acid inside the cell are isolated molecules for purposes of the present invention.

RB2/p130 gene expression is reduced or lost in cells having the RB2/p130 exon 1 mutations described above. As used herein, “expression,” with respect to the RB2/p130 gene means the realization of genetic information encoded in the gene to produce pRb2/p130 protein. “Expression” is thus used in its broadest sense, unless indicated to the contrary, to include either transcription or translation, as well as activity of the mature protein product of a gene. Thus, a reduction or absence of RB2/p130 RNA or pRb2/p130 protein, or a reduction or absence of RB2/p130 RNA or pRb2/p130 protein activity, in a test cell relative to a control cell would be considered “inhibition of pRb2/p130 expression.” As used herein, a “control” cell is a cell obtained from a subject who does not have, or is not suspected of having, cancer. Suitable techniques for measuring RB2/p130 gene expression are described in more detail below.

As demonstrated in the Examples below, loss of detectable RB2/p130 gene expression is seen in cancer cells that have homozygous mutations at nucleotides 178 and 259; see, e.g., Table 1, samples 3-5 and 9. A reduction in RB2/p130 gene expression is seen in cancer cells that have either a homozygous mutation at nucleotide 178 or nucleotide 259 of RB2/p130 exon 1, but not both; see, e.g., Table 1, samples 6-8 and 10. Where the RB2/p130 exon 1 mutations disclosed herein are heterozygous (even if both the nucleotide 178 and 259 mutations occur together), then expression of RB2/p130 is normal; see, e.g., Table 1, sample 2. Thus, the presence of the homozygous RB2/p130 exon 1 mutations described above inhibit RB2/p130 gene expression.

TABLE 1

Correlation between RB2/p130 expression level and RB2/p130
mutational and methylation status

| | | | | |
|---|---|---|---|---|
| RB2/p130 | | | | Methylation |
| Expression Level | Exon | Codon | Amino-add substitution | Status |

| | | | | | |
|---|---|---|---|---|---|
| Sample 1 (B/F) | +++ | | Wt | Wt | U1, U2, U3 |
| Sample 2 (B/F) | +++ | 1 | TCT-CCT (178) | SER-PRO (heterozygous) | U1, U2, U3 |
| | | | CCC-GCC (259) | PRO-ALA (heterozygous) | |
| Sample 3 (U/S) | − | 1 | TCT-CCT (178) | SER-PRO (homozygous) | M1, M2, M3 |
| | | | CCC-GCC (259) | PRO-ALA (homozygous) | |
| Sample 4 (U/S) | − | 1 | TCT-CCT (178) | SER-PRO (homozygous) | M1, M2, M3 |
| | | | CCC-GCC (259) | PRO-ALA (homozygous) | |
| | | 12 | GCA-GCG (1650) | Homozygous silent | |
| | | 21 | TAT-GAT (3178) | TYR-ASP (heteroaygous) | |
| Sample 5 (U/S) | − | 1 | TCT-CCT (178) | SER-PRO (homozygous) | M1, M2, M3 |
| | | | CCC-GCC (259) | PRO-ALA (homozygous) | |
| | | 12 | GCA-GCG (1650) | Homozygous silent | |
| | | 13 | GAA-CAA (1792) | GLU-GLN (homozygous) | |
| Sample 6 (U/S) | + | 1 | TCT-CCT (178) | SER-PRO (honmzygous) | U1, U2, M3 |
| | | | CCC-GCC (259) | PRO-ALA (heterozygous) | |
| | | 6 | GTT-ATT (928) | VAL-ILE (heterozygous) | |
| Sample 7 (B/S) | + | 1 | TCT-CCT (178) | SER-PRO (homozygous) | U1, U2, M3 |
| | | | CCC-GCC (259) | PRO-ALA (heterozygous) | |
| | | 12 | GCA-GCG (1650) | Homozygous silent | |
| Sample 8 (B/S) | + | 1 | TCT-CCT (178) | SER-PRO (homozygous) | U1, U2, M3 |
| | | | CCC-GCC (259) | PRO-ALA (heterozygous) | |
| | | 12 | GCA-GCG (1650) | Homozygous silent | |
| Sample 9 (B/S) | − | 1 | TCT-CCT (178) | SER-PRO (homozygous) | M1, M2, M3 |
| | | | CCC-GCC (259) | PRO-ALA (homozygous) | |
| | | 4 | TTT-TTG | PHE-LEU (heterozygous) | |
| | | 12 | GCA-GCG (1650) | Homozygous silent | |
| Sample 10 (B/S) | + | 1 | TCT-CCT (178) | SER-PRO (homozygous) | U1, U2, M3 |
| | | | CCC-GCC (259) | PRO-ALA (heterozygous) | |
| | | 12 | GCA-GCG (1650) | Homozygous silent | |
| | | 1 | TCT-CCT (178) | SER-PRO (homozygous) | |
| | | | CCC-GCC (259) | PRO-ALA (homozygous) | |
| Weri cell line | + | 4 | TAT-TGT (698) | TYR-CYS (heterozygous) | U1, U2, M3 |
| | | 12 | GCA-GCG (1650) | Homozygous silent | |
| | | 16 | CAG-CAC (2403) | GLN-HIS (heterozygous) | |
| | | | AGT-ATT (2411) | SER-ILE (heterozygous) | |
| | | | AGT-ATT (2549) | SER-ILE (heterozygous) | |
| Normal retina | +++ | | Wt | Wt | U1, U2, U3 |
| (three samples) | | | | | |
| Health donors | +++ | | Wt | Wt | |
| (15 donors) | | | | | |

Primary familiar (F) and sporadic (S) retinoblastoma bilateral (B) and unilateral (U) tumors, Weri-Rb1 cell line, normal retina samples and health donors were characterized by immunohistochemical analysis.
RB2/p130 expression level is indicated: normal expression +++; weak expression +; negative expression −.
For RB2/p130 mutational analysis, the nucleotide substitution, codon and exon numbers and amino-acid substitution are indicated. In the column of methylation status, the amplified fragments, obtained by MSP analysis, were reported.
U1, U2 and U3 indicate the presence of unmethylated DNA. M1, M2 and M3 indicate the presence of an methylated DNA region

As can be seen from Table 1, the presence of homozygous exon 1 mutations in the RB2/p130 gene is correlated with the methylation state of three CpG-rich regions of the RB2/p130 gene regions in or near exon 1. These three CpG-rich regions of the RB2/p130 gene are located in the genomic sequence. The first such region is from about nucleotide −95 to about +177, encompassing the promoter region immediately 5′ to the transcription start site and part of exon 1 (“Region 1”). The second such region is from about nucleotide +167 to about +302, encompassing most of exon 1 (“Region 2”). The third such region is from about nucleotide +287 to about +411, encompassing the 3′-end of exon 1 and the 5′-end of intron 1 (“Region 3”). The numbering of nucleotides in the RB2/p130 gene with respect to Region 1, Region 2 and Region 3 is with reference to the ATG transcription start site, in which the adenine or “A” of the start codon is designated zero, the nucleotide immediately 5′ of the A of the start codon is designated −1, and the nucleotide immediately 3′ of the A of the start codon is designated +1. Region 1, Region 2 and Region 3 are shown schematically in FIG. 2a.

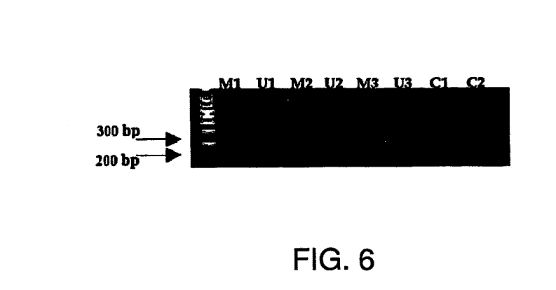

RB2/p136 gene expression is normal when Region 1, Region 2 and Region 3 are unmethylated. Methylation of Region 3 alone or of Regions 1 and 3 or Regions 2 and 3 result in a down regulation of RB2/p130 gene expression, and methylation of Regions 1, 2 and 3 renders RB2/p130 gene expression undetectable. See, e.g., Table 1 and FIGS. 2b and 6. Thus, methylation of at least Region 3 results in inhibition of RB2/p130 gene expression. Without wishing to be bound by any theory, it is believed that the homozygous mutations of the RB2/p130 gene described above establish a susceptibility to methylation of RB2/p130 genomic sequences, which in turn inhibits RB2/p130 gene expression. Again without wishing to be bound by any theory, it is believed that such an inhibition of RB2/p130 gene expression results in a loss or lowering of pRb2/p130 tumor-suppressor function in the cell, leading to the establishment or maintenance of tumorigenesis.

One skilled in the art would understand that methylation in Region 1, Region 2 or Region 3 of the RB2/p130 gene occurs at essentially each cytosine in the relevant RB2/p130 gene sequence which is followed by a guanosine in the 3′-direction; i.e., the sequence 5′-CG-3′, which is sometimes referred to a “CpG.” Thus, “methylation in Region 1, Region 2 and/or Region 3” means that essentially all the available 5′-CG-3′ methylation sites in a given region are methylated.

The homozygous RB2/p130 gene exon 1 mutations and/or the methylation of at least Region 3 have been detected in primary tumor cells and in cancer cell lines of different tissue histotype and embryonal origin. The invention thus provides a method of detecting tumor cells or diagnosing cancer in a subject, comprising the steps of obtaining a biological sample comprising test cells from a subject and obtaining nucleic acid from the test cells. The nucleic acid obtained from the test cells can be analyzed for mutations in exon 1 of the RB2/p130 gene, wherein the presence of homozygous mutations at nucleotides 178 and/or 259 of the RB2/p130 gene indicate that the test cells are tumor cells or that the subject has cancer. Alternatively, DNA obtained from the test cells can be analyzed the methylation status of the RB2/p130 gene, wherein methylation of at least Region 3 indicates that the test cells are tumor cells or that the subject has cancer.

The homozygous RB2/p130 gene exon 1 mutations and/or the methylation of at least Region 3 have also been detected in cells from normal-appearing tissue obtained from sites adjacent to a tumor, whereas no RB2/p130 gene exon 1 mutations are present in cells obtained from subjects with no tumors or from subjects with non-tumoral pathologies. The invention thus also provides a method of detecting cells which are predisposed to tumorigenesis, comprising obtaining a biological sample comprising test cells from a subject, wherein the test cells appear histologically or morphologically normal. Nucleic acid obtained from the test cells can be analyzed for the presence of RB2/p130 gene exon 1 mutations or the methylation status of the RB2/p130 gene, as described above. The presence of homozygous mutations at nucleotides 178 and/or 259 of the RB2/p130 gene or methylation of at least Region 3 indicates that the test cells are predisposed to tumorigenesis.

Tissue samples containing test cells for use in the present methods include blood samples, and can be obtained by standard techniques, such as drawing blood from a vein or artery, swabbing skin or mucosal membrane surfaces, punch or needle biopsy, surgical biopsy, and the like. Nucleic acid can then be obtained from the test cells using standard techniques, for determination of RB2/p130 exon 1 mutations or methylation levels. The nucleic acid obtained from the test cells can be DNA, RNA or both.

The presence of RB2/p130 exon 1 mutations can be detected in the nucleic acid obtained from the test cells by any suitable technique, for example, amplification of the relevant exon 1 regions in RB2/p130 RNA or DNA by polymerase chain reaction, or analysis of Southern blot hybridization of RB2/p130 DNA using probes specific for the exon 1 mutations.

Southern blot hybridization techniques are within the skill in the art. For example, DNA obtained from test cells can be digested with restriction endonucleases. This digestion generates restriction fragments of the genomic DNA, which can be separated by electrophoresis, for example on an agarose gel. The restriction fragments are then blotted Onto a hybridization membrane (e.g. nitrocellulose or nylon), and hybridized with labeled probes specific for the RB2/p130 mutations. Probe labeling and hybridization conditions suitable for detecting the exon 1 mutations can be readily determined by one of ordinary skill in the art. Suitable nucleic acid probes for Southern blot hybridization can be designed based upon the wild-type and mutant RB2/p130 cDNAs disclosed herein.

Methods for preparation of labeled DNA and RNA probes, and the conditions for hybridization thereof to target nucleotide sequences, are described in Molecular Cloning: A Laboratory Manual, J. Sambrook et al., eds., 2nd edition, Cold Spring Harbor Laboratory Press, 1989, Chapters 10 and 11, the disclosures of which are herein incorporated by reference. For example, nucleic acid probes can be labeled to high specific activity by either the nick translation method of Rigby et al. (1977), J. Mol. Biol. 113:237-251 or by the random priming method of Fienberg et al. (1983), Anal. Biochem. 132:6-13, the entire disclosures of which are herein incorporated by reference. Autoradiographic detection of hybridization can then be performed by exposing hybridized filters to photographic film. Where radionuclide labeling of DNA or RNA probes is not practical, the random-primer method can be used to incorporate non-radioactive labels such as the dTTP analogue 5-(N-(N-biotinyl-epsilon-aminocaproyl)-3-aminoallyl)deoxyuridine triphosphate into the probe molecule. The biotinylated probe oligonucleotide can be detected by reaction with biotin-binding proteins such as avidin, streptavidin, or anti-biotin antibodies coupled with fluorescent dyes or enzymes which produce color reactions.

Another suitable technique for determining whether the RB2/p130 exon 1 mutations are present is single strand conformational polymorphism or “SSCP,” for example as described in Orita et al. (1989), Genomics 5: 874-879 and Hayashi (1991), PCR Methods and Applic. 1: 34-38, the entire disclosures of which are herein incorporated by reference. The SSCP technique comprises amplifying a fragment of the gene of interest by PCR, denaturing the fragment and electrophoresing the two denatured single strands under non-denaturing conditions. The single strands assume a complex sequence-dependent intrastrand secondary structure that affects the strands electrophoretic mobility. Differences in the electrophoretic mobility of the single strands versus analogous single strands amplified from nucleic acid obtained from control cells indicates the presence of the RB2/p130 exon 1 mutations.

Preferably, RB2/p130 exon 1 mutations are detected by amplifying a fragment of these genes by polymerase chain reaction (PCR), and analyzing the amplified fragment by sequencing or by electrophoresis to determine if the mutation at nucleotide 178 or nucleotide 259 is present. Suitable reaction and cycling conditions for PCR amplification of DNA fragments can be readily determined by one of ordinary skill in the art. Exemplary PCR reaction and cycling conditions are given in the Examples, below.

Methods for determining the methylation pattern of the RB2/p130 gene are within the skill in the art, and representative techniques include methylation-specific PCR or “MSP,” for example as described in the Examples below.

Although the homozygous RB2/p130 gene exon 1 mutations described above result in an inhibition of RB2/p130 gene expression, some mutant pRb2/p130 protein can be produced. The presence of the RB2/p130 gene exon 1 mutations can therefore also be detected by obtaining protein from the test cells, and analyzing the protein for mutant pRb2/p130 proteins.

As described above, the exon 1 mutations produce mutant pRb2p130 proteins that contain either a Ser→Pro substitution at codon 37 (SEQ ID NO: 4) or a Pro→Ala substitution at codon 64 (SEQ ID NO: 6), or both a codon 37 Ser→Pro and a codon 64 Pro→Ala substitution (SEQ ID NO: 8). The presence of only a particular mutant pRb2/p130 protein in the test cells indicates that the RB2/p130 gene exon 1 mutation is homozygous, and that test cells are tumor cells or the subject has cancer. If the test cells are taken from tissue that is histologically or morphologically normal, and only a particular mutant pRb2/p130 protein is detected, then the test cells are predisposed to tumorigenesis. However, if normal pRb2/p130 protein is detected, or mutant pRb2/p130 protein of different types are found (i.e., some with only the codon 37 Ser→Pro substitution and some with only the codon 64 Pro→Ala substitution), then the test cell likely does not have a homozygous exon 1 RB2/p130 gene mutation.

Suitable techniques for detecting mutant pRb2/p130 proteins are within the skill in the art, and include electrophoretic separation and identification, peptide digestion and sequence analysis; and immunoassays such as radioimmunoassays, ELISA, “sandwich” immunoassays, gel diffusion precipitation reactions, in situ immunoassays, complement fixation assays, and immunoelectrophoretic assays.

A silent homozygous mutation in RB2/p130 exon 12 was identified in sporadic retinoblastoma tumors. This mutation is an A to G transition found at nucleotide 1650 of SEQ ID NO: 1. The cDNA sequence of RB2/p130 having the exon 12 A to G transition at nucleotide 1650 is given in SEQ ID NO: 9. The invention thus provides isolated nucleic acid sequences comprising the RB2/p130 exon 12 mutation described herein. This exon 12 mutation is specific for sporadic retinoblastoma, and its presence is therefore predictive of that tumor phenotype. In one embodiment, therefore, the invention provides a method for detecting sporadic retinoblastoma tumor cells or for diagnosing sporadic retinoblastoma in a subject. As described below, test cells can be obtained from a subject, and nucleic acid obtained from those cells. The nucleic acid can be analyzed for the presence of the exon 12 mutation described herein, for example using those techniques discussed in detail above. An exemplary technique for detecting the exon 12 mutation is given in the Examples below.

The demethylation of those portions of the RB2/p130 gene that have been methylated in cancer or pre-cancerous cells removes the inhibition of RB2/p130 gene expression, and restores pRb2/p130 tumor suppressor function in the cell. As used herein, a “pre-cancerous cell” is a cell which has a homozygous mutation at nucleotides 178 and/or 259 or has methylation of at least Region 3 of the RB2/p130 gene, but which is histologically or morphologically normal. Thus, the invention provides a method of treating cancer or of inhibiting tumorigenesis, in which the cells of the subject have a homozygous mutation at nucleotides 178 and/or 259 or have methylation of at least Region 3 of the RB2/p130 gene. The treatment method comprises the step of providing an effective amount of a demethylating agent to a subject who has, or is at risk for developing, cancer. Suitable DNA demethylating agents include DNA methyltransferase inhibitors such as 5-azacytidine (5-aza) and 5-Aza-2′-deoxycytidine (5-Aza-2dc). Methods for demethylating DNA and for determining the methylation pattern of the RB2/p130 gene are within the skill in the art, and representative techniques are given in the Examples below.

As used herein, “inhibiting tumorigenesis” means that the conversion of a pre-cancerous cell from a histologically or morphologically normal state into a state which is histologically or morphologically classified as neoplastic or cancerous. As used herein, an “effective amount of a demethylating agent” is an amount sufficient to inhibit the addition of or remove methyl groups from at least Region 1 and/or Region 2, and preferably Region 1, Region 2 and Region 3, of the RB2/p130 gene, or to remove the transcriptional inhibition of the RB2/p130 gene a cell. As used herein, an “effective amount” of a demethylating agent can also be an amount sufficient to inhibit proliferation of a tumor cell.

Test and control cells for use in determining levels of RB2/p130 expression at the RNA or protein level can be obtained by standard techniques as described above, such as by collection of blood from a vein or artery, punch or needle biopsy, surgical biopsy, and the like. The RB2/p130 RNA or pRb2/p130 protein can then be obtained from the test and control cells using standard techniques, for determination of RB2/p130 expression levels. Alternatively, the levels RB2/p130 expression in a test sample can be compared to average levels of RB2/p130 gene expression previously obtained for a population of normal control subjects. As used herein, a “normal control subject” is a subject who does not have, or is not suspected of having, cancer.

Suitable techniques for determining the level of RNA transcripts of a particular gene in cells are within the skill in the art. According to one such method, total cellular RNA can be purified from cells by homogenization in the presence of nucleic acid extraction buffer, followed by centrifugation. Nucleic acids are then precipitated, and DNA is removed by treatment with DNase. The RNA molecules are then separated by gel electrophoresis on agarose gels according to standard techniques, and transferred to nitrocellulose or other suitable filters by, e.g., the so-called “Northern” blotting technique. The RNA is immobilized on the filters by heating. Detection and quantification of specific RNA is accomplished using appropriately labeled DNA or RNA probes complementary to the RNA in question. See, for example, Molecular Cloning: A Laboratory Manual, J. Sambrook et al., eds., 2nd edition, Cold Spring Harbor Laboratory Press, 1989, Chapter 7, the entire disclosure of which is incorporated by reference.

Autoradiographic detection of probe hybridization to RB2/p130 RNA can be performed by exposing hybridized filters to photographic film. Densitometric scanning of the photographic films exposed by the hybridized filters provides an accurate measurement of RNA transcript levels. Alternatively, RNA transcript levels can be quantified by computerized imaging of the hybridization filter, for example with the Molecular Dynamics 400-B2D Phosphorimager available from Amersham Biosciences, Piscataway, N.J.

In addition to blotting hybridization techniques, detection of RNA transcripts from a given gene can be carried out by in situ hybridization. This technique requires fewer cells than the Northern blotting technique, and involves depositing whole cells onto a microscope slide or cover slip and probing the nucleic acid content of the cell with a solution containing radioactive or otherwise labeled cDNA or cRNA probes. The practice of the in situ hybridization technique is described in more detail in U.S. Pat. No. 5,427,916, the entire disclosure of which is incorporated herein by reference.

The number of RB2/p130 RNA transcripts in test or control cells can also be determined by reverse transcription of RB2/p130 RNA transcripts, followed by amplification by polymerase chain reaction (RT-PCR). The levels of RB2/p130 RNA transcripts can be quantified in comparison with an internal standard; for example, by comparison to levels of RNA produced from a “housekeeping” gene present in the same sample. A suitable “housekeeping” gene for use as an internal standard includes myosin, β-actin or glyceraldehyde-3-phosphate dehydrogenase (GAPDH). Methods of performing quantitative RT-PCR and variations thereon are within the skill in the art.

RB2/p130 gene expression can also be determined by measuring the level of pRb2/p130 protein in a test cells versus a control cells. Suitable techniques for measuring pRb2/p130 protein levels are known in the art, and include electrophoretic separation and identification, peptide digestion, and sequence analysis; and immunoassays such as radioimmunoassays, ELISA (enzyme linked immunosorbent assay), “sandwich” immunoassays, gel diffusion precipitation reactions, in situ immunoassays, complement fixation assays, and immunoelectrophoretic assays.

One skilled in the art can readily determine an effective amount of a demethylating agent to be administered to a given subject, by taking into account factors such as the size and weight of the subject; the extent of the tumor growth or disease penetration; the age, health and sex of the subject; the route of administration; and whether the administration is regional (e.g., local) or systemic.

For example, an effective amount of demethylating agent can comprise from about 5-3000 μg compound/kg of body weight, preferably between about 700-1000 μg compound/kg of body weight, and more preferably greater than about 1000 μg compound/kg of body weight. It is contemplated that greater or lesser amounts of a demethylating agent can be administered to a subject An effective amount of the compounds of the invention can also be based on the approximate weight of a tumor mass to be treated. The approximate weight of a tumor mass can be determined by calculating the approximate volume of the mass, wherein one cubic centimeter of volume is roughly equivalent to one gram. An effective amount of the demethylating agent based on the weight of a tumor mass can be at least about 10 μg/gram of tumor mass, and is preferably between about 10-500 μg/gram of tumor mass. More preferably, the effective amount is at least about 60 μg/gram of tumor mass. Particularly preferably, the effective amount is at least about 100 μg/gram of tumor mass. It is preferred that an effective amount of a demethylating agent based on the weight of the tumor mass be injected directly into the tumor.

Demethylating agents can be administered to a subject by any means suitable for exposing cancer or precancerous cells to the agent. For example, the agent can be administered by parenteral or enteral administration routes. Suitable enteral administration routes include oral, rectal, or intranasal delivery. Suitable parenteral administration routes include intravascular administration (e.g., intravenous bolus injection, intravenous infusion, intra-arterial bolus injection, intra-arterial infusion and catheter instillation into the vasculature); peri-tumoral and intra-tumoral injection; intramuscular injection; subcutaneous injection or deposition including subcutaneous infusion (such as by osmotic pumps); direct application to the tissue of interest, for example by a catheter or other placement device (e.g., a suppository or an implant comprising a porous, non-porous, or gelatinous material); and inhalation. Preferably, a demethylating agent is administered by injection or infusion, more preferably by direct injection into a tumor.

One skilled in the art can also readily determine an appropriate dosage regimen for administering demethylating agents to a subject. For example, the agent can be administered to the subject once, for example as a single injection or deposition. Alternatively, the agent can be administered once or twice daily to a subject for a period of from about three to about twenty-eight days, more preferably from about seven to about ten days. In a preferred dosage regimen, the agent is injected once a day for seven days. Where a dosage regimen comprises multiple administrations, it is understood that the effective amount of the demethylating agent administered to the subject can comprise the total amount of the agent administered over the entire dosage regimen.

Demethylating agents can be formulated as pharmaceutical compositions or medicaments prior to administering to a subject, according to techniques known in the art. Thus, the use of a demethylating agent for the production of a pharmaceutical composition or medicament for the treatment of cancer is specifically contemplated by the present invention.

As used herein, “pharmaceutical formulations” or “medicaments” include formulations for human and veterinary use. Pharmaceutical compositions or medicaments of the present invention for parenteral administration are characterized as being at least sterile and pyrogen-free. Methods for preparing pharmaceutical compositions and medicaments of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical formulations or medicaments comprise at least one demethylating agent (e.g., 0.1 to 90% by weight), or a physiologically acceptable salt thereof, mixed with a physiologically acceptable carrier. Preferred physiologically acceptable carriers are water, buffered water, normal saline, 0.4% saline, 0.3% glycine, hyaluronic acid and the like.

Pharmaceutical compositions or medicaments of the invention can also comprise conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like. For example, a solid pharmaceutical composition for oral administration can comprise any of the carriers and excipients listed above and 10-95%, preferably 25%-75%, of one or more demethylating agent. A pharmaceutical composition or medicament for aerosol (inhalational) administration can comprise 0.01-20% by weight, preferably 1%/-10% by weight, of demethylating agent encapsulated in a liposome, and propellant. A carrier can also be included as desired; e.g., lecithin for intranasal delivery.

The ICBP90 protein and other proteins which comprise a pRb2/p130 complex (including pRb2/p130 , PAI-2, HDAC1, DNMT1, p300, SUV39H1, and E2F and other transcription factors) or DNA methylases (e.g., DNMT1, DNMT3a, DNMT3b) can also be a therapeutic targets for treating cancer. For ease of illustration, the following discussion will focus on ICBP90. However, it is understood that the following discussion also applies to any of the proteins involved in the ICBP90-pRb2/p130 complex regulation of gene expression, such as those listed in the first sentence of this paragraph.

Thus, the direct or indirect inhibition of ICBP90 protein activity can affect the formation of pRb2/p130 protein complexes, and result in the expression of tumor suppressor genes which might otherwise be down-regulated in certain cancerous or precancerous states. Inhibition of ICBP90 protein can be achieved by any suitable techniques known in the art, such as by introducing an antibody, aptamer or other molecule which binds to ICBP90 and prevents ICBP90 from binding to methylated sites on DNA and initiating the formation of pRb2/p130 complexes. ICBP90 protein can also be inhibited by specifically preventing transcription or translation of ICBP90 RNA.

ICBP90 expression can be inhibited by any suitable technique known to one of ordinary skill in the art. For example, ICBP90 expression can be inhibited by administering antisense oligonucleotides designed to target the ICBP90 mRNA (see, e.g., GenBank Accession No. AB126777, the entire disclosure of which is herein incorporated by reference). The ICBP90 target can be single-stranded or double stranded DNA or RNA; however, single-stranded DNA or RNA targets are preferred, with single-stranded mRNA targets being particularly preferred. It is understood that the target to which the ICBP90 antisense oligonucleotides of the invention are directed include allelic forms of ICBP90.

There is substantial guidance in the literature for selecting particular sequences for antisense oligonucleotides given a knowledge of the sequence of the target polynucleotide; e.g., Peyman and Ulmann, 1990, Chemical Reviews, 90, 543; Crooke, 1992, Ann. Rev. Pharmacal. Toxicol., 32, 329; and Zamecnik and Stephenson, Proc. Natl. Acad. Sci., 75, 280, the entire disclosures of which are herein incorporated by reference. Preferably, the sequences of ICBP90 antisense compounds are selected such that the G-C content is at least 60%. Preferred ICBP90 mRNA targets include the 5′ cap site, tRNA primer binding site, the initiation codon site, the mRNA donor splice site, and the mRNA acceptor splice site; see, e.g. Goodchild et al., U.S. Pat. No. 4,806,463, the entire disclosure of which is herein incorporated by reference.

Where the target polynucleotide comprises a ICBP90 mRNA transcript, oligonucleotides complementary to any portion of the transcript are, in principle, effective for inhibiting translation and capable of inducing the effects herein described.

It is believed that translation is most effectively inhibited by blocking the mRNA at a site at or near the initiation codon. Thus, oligonucleotides complementary to the 5′-region of the ICBP90 mRNA transcript are preferred. Oligonucleotides complementary to the ICBP90 mRNA, including the initiation codon (the first codon at the 5′ end of the translated portion of the pRb2/p130 transcript), or codons adjacent the initiation codon, are preferred.

While antisense oligonucleotides complementary to the 5′-region of the ICBP90 transcript are preferred, particularly the region including the initiation codon, it should be appreciated that useful antisense oligomers are not limited to those complementary to the sequences found in the translated portion of the mRNA transcript, but also include oligomers complementary to nucleotide sequences contained in, or extending into, the 5′- and 3′-untranslated regions of the mRNA transcript.

Antisense oligonucleotides of the invention can comprise any polymeric compound capable of specifically binding to a target polynucleotide by way of a regular pattern of monomer-to-nucleoside interactions, such as Watson-Crick type of base pairing, Hoogsteen or reverse Hoogsteen types of base pairing, or the like. Antisense compounds of the invention can also contain pendent groups or moieties, either as part of or separate from the basic repeat unit of the polymer, to enhance specificity, nuclease resistance, delivery, or other property related to efficacy; e.g., cholesterol moieties, duplex intercalators such as acridine, poly-L-lysine, “end-capping” with one or more nuclease-resistant linkage groups such as phosphorothioate, and the like.

For example, it is known that enhanced lipid solubility and/or resistance to nuclease digestion results by substituting an alkyl group or alkoxy group for a phosphase oxygen in the internucleotide phosphodiester linkage to form an alkylphosphonate oligonucleoside or alkylphosphotriester oligonucleotide. Non-ionic oligonucleotides such as these are characterized by increased resistance to nuclease hydrolysis and/or increased cellular uptake, while retaining the ability to form stable complexes with complementary nucleic acid sequences. The alkylphosphonates, in particular, are stable to nuclease cleavage and soluble in lipid. The preparation of alkylphosphonate oligonucleosides is disclosed in Ts'o et al., U.S. Pat. No. 4,469,863, the entire disclosure of which is herein incorporated by reference.

Preferably, nuclease resistance is conferred on the antisense compounds of the invention by providing nuclease-resistant internucleosidic linkages. Many such linkages are known in the art; e.g., phosphorothioate: Zon and Geyser, 1991, Anti Cancer Drug Design, 6:539; Stec et al., U.S. Pat. No. 5,151,510; Hirschbein, U.S. Pat. No. 5,166,387; Bergot, U.S. Pat. No. 5,193,885; phosphorodithioates: Marshall et al., 1993, Science, 259, 1564; Caruthers and Nielsen, International application PCT/US89/02293; phosphoramidates, e.g., —OP(═O)(NR1R2)—O— with R1 and R2 hydrogen or C—C3 alkyl; Jager et al., 1988, Biochemistry, 27, 7237; Froehler et al., International application PCT/US90/03138; peptide nucleic acids: Nielsen et al., 1993, Anti-Cancer Drug Design, 8, 53; International application PCT/EP92/01220; methylphosphonates: Miller et al., U.S. Pat. No. 4,507,433, Ts'o et al., U.S. Pat. No. 4,469,863; Miller et al., U.S. Pat. No. 4,757,055; and P-chiral linkages of various types, especially phosphorothioates, Stec et al., European patent application 506,242 (1992) and Lesnikowski, Bioorganic Chemistry, 21, 127. Additional nuclease linkages include phosphoroselenoate, phosphorodiselenoate, phosphoroanilothioate, phosphoranilidate, alkylphosphotriester such as methyl- and ethylphosphotriester, carbonate such as carboxymethyl ester, carbamate, morpholino carbamate, 3′-thioformacetal, silyl such as dialkyl(C—Ce)— or diphenylsilyl, sulfamate ester, and the like. Such linkages and methods for introducing them into oligonucleotides are described in many references; e.g., reviewed generally by Peyman and Ulmann, 1990, Chemical Reviews 90:543; Milligan et al., 1993, J; Med. Chem., 36, 1923; Matteucci et al., International application PCT/US91/06855. The entire disclosures of all documents referred to in this paragraph are herein incorporated by reference.

Resistance to nuclease digestion may also be achieved by modifying the internucleotide linkage at both the 5′ and 3′ termini with phosphoroamidites according to the procedure of Dagle et al., 1990, Nucl. Acids Res. 18, 4751, the entire disclosure of which is herein incorporated by reference.

Preferably, phosphorus analogs of the phosphodiester linkage are employed in the compounds of the invention, such as phosphorothioate, phosphorodithioate, phosphoramidate, or methylphosphonate. More preferably, phosphorothioate is employed as the nuclease resistant linkage.

Phosphorothioate oligonucleotides contain a sulfur-for-oxygen substitution in the internucleotide phosphodiester bond. Phosphorothioate oligonucleotides combine the properties of effective hybridization for duplex formation with substantial nuclease resistance, while retaining the water solubility of a charged phosphate analogue. The charge is believed to confer the property of cellular uptake via a receptor (see Loke et al., 1989, Proc. Natl. Acad. Sci., 86, 3474, the entire disclosure of which is herein incorporated by reference).

It is understood that in addition to the preferred linkage groups, antisense compounds of the invention can comprise additional modifications; e.g., boronated bases (see, e.g., Spielvogel et al., U.S. Pat. No. 5,130,302); cholesterol moieties (see, e.g., Shea et al., 1990, Nucl. Acids Res., 18, 3777 or Letsinger et al., 1989, Proc. Natl. Acad. Sci. USA, 86, 6553); and 5-propynyl modification of pyrimidines (see, e.g. Froehler et al., 1992, Tetrahedron Lett., 33, 5307). The entire disclosures of all documents referred to in this paragraph are herein incorporated by reference.

Antisense compounds of the invention can be synthesized by conventional means on commercially available automated DNA synthesizers; e.g., an Applied Biosystems (Foster City, Calif.) model 380B, 392 or 394 DNA/RNA synthesizer. Preferably, phosphoramidite chemistry is employed, e.g., as disclosed in the following references: Beaucage and Iyer, 1992, Tetrahedron, 48, 2223; Molko et al., U.S. Pat. No. 4,980,460; Koster et al., U.S. Pat. No. 4,725, 677; Caruthers et al., U.S. Pat. Nos. 4,415,732; 4,458,066; and 4,973,679, the entire disclosures of which are herein incorporated by reference.

In embodiments where triplex nucleic acid formation is desired, there are constraints on the selection of target sequences. Generally, third strand association via Hoogsteen type of binding is most stable along homopyrimidine-homopurine tracks in a double stranded target. Usually, base triplets form in T-A*T or C-G*C motifs (where “−” indicates Watson-Crick pairing and “*” indicates Hoogsteen type of binding); however, other motifs are also possible. For example, Hoogsteen base pairing permits parallel and antiparallel orientations between the third strand (the Hoogsteen strand) and the purine-rich strand of the duplex to which the third strand binds, depending on conditions and the composition of the strands. There is extensive guidance in the literature for selecting appropriate sequences, orientation, conditions, nucleoside type (e.g., whether ribose or deoxyribose nucleosides are employed), base modifications (e.g., methylated cytosine, and the like) in order to maximize, or otherwise regulate, triplex stability as desired in particular embodiments; see, e. g., Roberts et al., 1991, Proc. Natl. Acad. Sci. USA, 88, 9397; Roberts et al., 1992, Science, 258, 1463; Distefano et al., 1993, Proc. Natl. Acad. Sci. USA, 90, 1179; Merguy et al., Biochemistry, 30, 9791-9798 (1992); Cheng et al., J. Am. Chem. Soc., 114:4465-4474 (1992); Beat and Dervan, Nucleic Acids Research, 20:2773-2776 (1992); Beat and Dervan, J. Am. Chem. Soc., 114:4976-4982; Giovannangeli et al., Proc. Natl. Acad. Sci., 89:8631-8635 (1992); Moser and Dervan, Science, 238:645-650 (1987); McShan et al., J. Biol. Chem., 267: 5712-5721 (1992); Yoon et al., Proc. Natl. Acad. Sci., 89:3840-3844 (1992); and Blume et al., Nucleic Acids Research, 20:1777-1784 (1992), the entire disclosures of which are herein incorporated by reference.

The length of the antisense oligonucleotides should be sufficiently large to ensure that specific binding will take place only at the desired target polynucleotide and not at other fortuitous sites, as explained in many references; e.g., Rosenberg et al., International application PCT/US92/05305; or Szostak et al., 1979, Meth. Enzymol., 68, 419, the entire disclosures of which are herein incorporated by reference. The upper range of the length is determined by several factors, including the inconvenience and expense of synthesizing and purifying oligomers greater than about 30-40 nucleotides in length, the greater tolerance of longer oligonucleotides for mis matches than shorter oligonucleotides, whether modifications to enhance binding or specificity are present, whether duplex or triplex binding is desired, and the like.

Usually, antisense compounds of the invention have lengths in the range of about 12 to nucleotides. More preferably, antisense compounds of the invention have lengths in the range of about 15 to 40 nucleotides; and most preferably, they have lengths in the range of about 18 to 30 nucleotides.

In general, the antisense oligonucleotides used in the practice of the present invention will have a sequence which is completely complementary to a selected portion of the target polynucleotide. Absolute complementarily is not however required, particularly in larger oligomers. Thus, reference herein to a “nucleotide sequence complementary to” a target polynucleotide does not necessarily mean a sequence having 100% complementarily with the target segment. In general, any oligonucleotide having sufficient complementarily to form a stable duplex with the target (e.g., the ICBP90 mRNA) is suitable. Stable duplex formation depends on the sequence and length of the hybridizing oligonucleotide and the degree of complementarity with the target polynucleotide. Generally, the larger the hybridizing oligomer, the more mismatches may be tolerated. More than one mismatch probably will not be tolerated for antisense oligomers of less than about 21 nucleotides. One skilled in the art can readily determine the degree of mismatching which may be tolerated between any given antisense oligomer and the target sequence, based upon the melting point, and therefore the thermal stability, of the resulting duplex.

Preferably, the thermal stability of hybrids formed by the antisense oligonucleotides of the invention are determined by way of melting, or strand dissociation, curves. The temperature of fifty percent strand dissociation is taken as the melting temperature, Tm, which, in turn, provides a convenient measure of stability. Tm measurements are typically carried out in a saline solution at neutral pH with target and antisense oligonucleotide concentrations at between about 1.0-2.0 uM. Typical conditions are as follows: 150 mM NaCl and 10 mM MgCl2 in a 10 mM sodium phosphate buffer (pH 7.0) or in a 10 M Tris-HCl buffer (pH 7.0). Data for melting curves are accumulated by heating a sample of the antisense oligonucleotide/target polynucleotide complex from room temperature to about 85-90° C. As the temperature of the sample increases, absorbance of 260 nm light is monitored at 1° C. intervals, e.g., using a Cary (Australia) model 1E or a Hewlett-Packard (Palo Alto, Calif.) model HP 8459 UV/VIS spectrophotometer and model HP 89100A temperature controller, or like instruments. Such techniques provide a convenient means for measuring and comparing the binding strengths of antisense oligonucleotides of different lengths and compositions.

ICBP90 expression can also be inhibited by “RNA interference” or “RNAi.” RNAi is a method of post-transcriptional gene regulation that is conserved throughout many eukaryotic organisms. RNAi is induced by short (i.e., >30 nucleotide) double stranded RNA (“dsRNA”) molecules (Fire A et al. (1998), Nature 391: 806-811).

These short dsRNA molecules, called “short or small interfering RNA” or “siRNA,” cause the destruction of RNAs which share sequence homology with the siRNA to within one nucleotide resolution Elbashir S M et al. (2001), Genes Dev, 15: 188-200). It is believed that the siRNA and the targeted RNA bind to an “RNA-induced silencing complex” or “RISC”, which cleaves the targeted RNA. The siRNA is apparently recycled much like a multiple-turnover enzyme, with one siRNA molecule capable of inducing cleavage of approximately 1000 RNA molecules. siRNA-mediated RNAi degradation of an RNA is therefore more effective than currently available technologies for inhibiting expression of a target gene. The specificity of siRNA induced RNAi allows the targeting of subject-specific target (e.g., ICBP90) alleles, so that “personalized treatment” of the subject's breast cancer can be performed. The siRNA of the invention can comprise short double-stranded RNA from about 17 nucleotides to about 29 nucleotides in length, preferably from about 19 to about 25 nucleotides in length, that are targeted to the particular RNA (e.g., ICBP90 RNA). The siRNA comprises a sense RNA strand and a complementary antisense RNA strand annealed together by standard Watson-Crick base-pairing interactions hereinafter “base-paired”). As is described in more detail below, the sense strand comprises a nucleic acid sequence which is identical to a target sequence contained within the target RNA.

The sense and antisense strands of the present siRNA can comprise two complementary, single-stranded RNA molecules or can comprise a single molecule in which two complementary portions are base-paired and are covalently linked by a single-stranded “hairpin” area. Without wishing to be bound by any theory, it is believed that the hairpin area of the latter type of siRNA molecule is cleaved intracellularly by the “Dicer” protein (or its equivalent) to form a siRNA of two individual base-paired RNA molecules (see Tuschl, T. (2002), supra).

The siRNA of the invention can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribonucleotides. One or both strands of the siRNA of the invention can also comprise a 3′ overhang. As used herein, a “3′ overhang” refers to at least one unpaired nucleotide extending from the 3′-end of an RNA strand.

Thus, the siRNA of the invention can comprise at least one 3′ overhang of from 1 to about 6 nucleotides (which includes ribonucleotides or deoxynucleotides) in length, preferably from 1 to about 5 nucleotides in length, more preferably from 1 to about 4 nucleotides in length, and particularly preferably from about 2 to about 4 nucleotides in length.

In the embodiment in which both strands of the siRNA molecule comprise a 3′ overhang, the length of the overhangs can be the same or different for each strand. In a most preferred embodiment, the 3′ overhang is present on both strands of the siRNA, and is 2 nucleotides in length. For example, each strand of the siRNA of the invention can comprise 3′ overhangs of dithymidylic acid (“TT”) or diuridylic acid (uu).

In order to enhance the stability of the present siRNA, the 3′ overhangs can be also stabilized against degradation. In one embodiment, the overhangs are stabilized by including purine nucleotides, such as adenosine or guanosine nucleotides.

Alternatively, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine significantly enhances the nuclease resistance of the 3′ overhang in tissue culture medium.

The siRNA of the invention can be targeted to any stretch of approximately 19-25 contiguous nucleotides (the “target sequence”) in the target RNA. Generally, a target sequence on the target RNA can be selected from a given cDNA sequence corresponding to the target RNA, preferably beginning 50 to 100 nt downstream (i.e., in the 3′ direction) from the start codon. The target sequence can, however, be located in the 5′ or 3′ untranslated regions, or in the region nearby the start codon.

Techniques for selecting target sequences for siRNA's are within the skill in the art and are given, for example, in Tuschl T et al., “The siRNA User Guide,” revised Oct. 11, 2002, the entire disclosure of which is herein incorporated by reference. “The siRNA User Guide” is available on the world wide web at a website maintained by Dr. Thomas Tuschl, Department of Cellular Biochemistry, AG 105, Max-Planck-Institute for Biophysical Chemistry, 37077 Gottingen, Germany, and can be found by accessing the website of the Max Planck Institute and searching with the keyword “siRNA.” Thus, the sense strand of the present siRNA comprises a nucleotide sequence identical to any contiguous stretch of about 19 to about 25 nucleotides in the target RNA.

The siRNA of the invention can be obtained using a number of techniques known to those of skill in the art. For example, the siRNA can be chemically synthesized or recombinantly produced using methods known in the art, such as the Drosophila in vitro system described in U.S. published application 2002/0086356 of Tuschl et al., the entire disclosure of which is herein incorporated by reference.

Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

Alternatively, siRNA can also be expressed from recombinant circular or linear DNA plasmids using any suitable promoter. Suitable promoters for expressing siRNA of the invention from a plasmid include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the siRNA in a particular tissue or in a particular intracellular environment.

The siRNA expressed from recombinant plasmids can either be isolated from cultured cell expression systems by standard techniques, or can be expressed intracellularly. The use of recombinant plasmids to deliver siRNA of the invention to cells in vivo is discussed in more detail below.

An siRNA of the invention can also be expressed from a recombinant plasmid either as two separate, complementary RNA molecules, or as a single RNA molecule with two complementary regions.

Selection of plasmids suitable for expressing siRNA of the invention, methods for inserting nucleic acid sequences for expressing the siRNA into the plasmid, and methods of delivering the recombinant plasmid to the cells of interest are within the skill in the art. See, for example Tuschl, T. (2002), Nat. Biotechnol, 20: 446448; Brummelkamp T R et al. (2002), Science 296: 550-553; Miyagishi M et al. (2002), Nat. Biotechnol. 20: 497-500; Paddison P J et al. (2002), Genes Dev. 16: 948-958; Lee N S et al. (2002), Nat. Biotechnol. 20: 500-505; and Paul C P et al. (2002), Nat. Biotechnol. 20: 505-508, the entire disclosures of which are herein incorporated by reference.

The siRNA of the invention can also be expressed from recombinant viral vectors intracellularly in vivo. The recombinant viral vectors of the invention comprise sequences encoding the siRNA of the invention and any suitable promoter for expressing the siRNA sequences. Suitable promoters include, for example, the U6 or Hi RNA pot III promoter sequences and the cytomegalovirus promoter. Selection of other suitable promoters is within the skill in the art. The recombinant viral vectors of the invention can also comprise inducible or regulatable promoters for expression of the siRNA in a particular tissue or in a particular intracellular environment. The use of recombinant viral vectors to deliver siRNA of the invention to cells in vivo is discussed in more detail below.

siRNA of the invention can be expressed from a recombinant viral vector either as two separate, complementary RNA molecules, or as a single RNA molecule. Any viral vector capable of accepting the coding sequences for the siRNA molecule(s) to be expressed can be used, for example vectors derived from adenovirus (AV); adeno-associated virus (AAV); retroviruses (e.g. lentiviruses (LV), Rhabdoviruses, murine leukemia virus); herpes virus, and the like. The tropism of the viral vectors can also be modified by pseudotyping the vectors with envelope proteins or other surface antigens from other viruses. For example, an AAV vector of the invention can be pseudotyped with surface proteins from vesicular stomatitis virus (VSV), rabies, Ebola, Mokola, and the like.

Selection of recombinant viral vectors suitable for use in the invention, methods for inserting nucleic acid sequences for expressing the siRNA into the vector, and methods of delivering the viral vector to the cells of interest are within the skill in the art. See, for example, Dornburg R (1995), Gene Therap. 2: 301-310; Eglitis M A (1988), Biotechniques 6: 608-614; Miller A D (1990), Hum Gene Therap. 1: 5-14; Anderson W E (1998), Nature 392: 25-30; and Rubinson D A et al., Nat Genet. 33: 401-406, the entire disclosures of which are herein incorporated by reference.

Preferred viral vectors are those derived from AV and AAV. In a particularly preferred embodiment, the siRNA of the invention is expressed as two separate, complementary single-stranded RNA molecules from a recombinant AAV vector comprising, for example, either the U6 or Hi RNA promoters, or the cytomegalovirus (CMV) promoter. A suitable AV vector for expressing the siRNA of the invention, a method for constructing the recombinant AV vector, and a method for delivering the vector into target cells, are described in Xia H et al. (2002), Nat. Biotech. 20: 1006 1010. Suitable AAV vectors for expressing the siRNA of the invention, methods for constructing the recombinant AV vector, and methods for delivering the vectors into target cells are described in Samulski R et al. (1987), J. Virol. 61: 3096-3101; Fisher K J et al. (1996), J: Virol., 70: 520-532, Samulski R et al. (1989), J. Virol. 63: 3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference.

ICBP90 expression can also be inhibited at the protein level by compounds such as anti-ICBP90 antibodies and anti-ICBP90 aptamers. Anti-ICBP90 antibodies can be generated using the ICBP90 amino acid sequence, for example as provided in GenBank Accession No. AB 126777, supra, or immunogenic fragments thereof, by standard techniques. Antibodies can also be generated from ICBP90 protein isolated from a given subject or expressed from a ICBP90 cDNA isolated from a given subject.

Anti-ICBP90 antibodies can comprise a monoclonal antibody, a polyclonal antibody or an antibody fragment that is capable of binding an epitope of ICBP90 protein. Such antibodies include chimeric, single chain, and humanized antibodies, as well as Fab fragments and the products of an Fab expression library.

Polyclonal anti-ICBP90 antibodies can be produced by immunizing an animal with substantially pure ICBP90 protein or an immunogenic fragment thereof, using techniques well-known in the art. Antibody fragments, such as Fab antibody fragments, which retain some ability to selectively bind to the antigen of the antibody from which they are derived, can be made using well known methods in the art. Such methods are generally described in U.S. Pat. No. 5,876,997, the entire disclosure of which is incorporated herein by reference.

Monoclonal anti-ICBP90 antibodies can be prepared using the method of Mishell, BB et al., Selected Methods In Cellular Immunology, (Freeman WH, ea.) San Francisco, 1980, the entire disclosure of which is herein incorporated by reference. Briefly, a peptide is used to immunize spleen cells of Balb/C mice. The immunized spleen cells are fused with myeloma cells. Fused cells containing spleen and myeloma cell characteristics are isolated by growth in HAT medium, a medium which kills both parental cells, but allows the fused products to survive and grow.

Compounds which inhibit ICBP90, pRb2/p130 complex or DNA methylase proteins can be formulated into pharmaceutical compositions, as described above for DNA demethylating agents. Effective amounts of such compounds, or pharmaceutical compositions thereof, can be used to treat cancer or inhibiting proliferation of tumor cells. As used herein, an “effective amount of a compound which inhibits ICBP90, pRb2/p130 complex or DNA-methylase proteins” is that amount sufficient to inhibit proliferation of a tumor cell.

Effective amounts, dosage ranges and dosage regimens for inhibitors of ICBP90, pRb2/p130 complex or DNA methylase proteins can be readily determined by those of skill in the art, for example by taking into account the size, health, age, sex and disease penetration of a subject, and observing the amelioration of symptoms over the course of administering such inhibitors to a subject. Such compounds and pharmaceutical compositions thereof can be administered to a subject as described above for DNA demethylating agents.

The present methods can be used to detect tumors cells from or diagnose cancers, or inhibit the proliferation of tumor cells, of at least the following histologic subtypes: sarcoma (cancers of the connective and other tissue of mesodermal origin); melanoma (cancers deriving from pigmented melanocytes); carcinoma (cancers of epithelial origin); adenocarcinoma (cancers of glandular epithelial origin); cancers of neural origin (glioma/glioblastoma and astrocytoma); and hematological neoplasias, such as leukemias and lymphomas (e.g., acute lymphoblastic leukemia and chronic myelocytic leukemia).

The present methods can be used to detect tumors cells from or diagnose cancers, or inhibit the proliferation of tumor cells, having their origin in at least the following organs or tissues, regardless of histologic subtype: breast; tissues of the male and female urogenital system (e.g., ureter, bladder, prostate, testis, ovary, cervix, uterus, vagina); lung; tissues of the gastrointestinal system (e.g., stomach, large and small intestine, colon, rectum); exocrine glands such as the pancreas and adrenals; tissues of the mouth and esophagus; brain and spinal cord; kidney (renal); pancreas; hepatobiliary system (e.g. liver, gall bladder); lymphatic system; smooth and striated muscle; bone and bone marrow; skin; and tissues of the eye (e.g., retinoblastomas).

The present methods can be used to detect tumor cells from or diagnose cancers or tumors, or inhibit the proliferation of tumor cells, from tumors in any prognostic stage of development, for example as measured by the “Overall Stage Groupings” (also called “Roman Numeral”) or the “Tumor, Nodes, and Metastases” (TNM) staging systems. Appropriate prognostic staging systems and stage descriptions for a given cancer are known in the art, for example as described in the National Cancer Institute's “CancerNet” Internet website.

As used herein, to “inhibit the proliferation of tumor cell” means to kill the tumor cell, or permanently or temporarily arrest the growth of the tumor cell. Inhibition of tumor cell proliferation can be inferred if the number of tumor cells in the subject remains constant or decreases after administration of a compound or pharmaceutical composition of the invention. An inhibition of tumor cell proliferation can also be inferred if the absolute number of tumor cells increases, but the rate of tumor growth decreases. The number of tumor cells in a subjects body can be determined by direct measurement, or by estimation from the size of primary or metastatic tumor masses. The size of a tumor mass can be ascertained, for example, by direct visual observation or by diagnostic imaging methods such as X-ray, magnetic resonance imaging, ultrasound, and scintigraphy. Such diagnostic imaging methods can be employed with or without contrast agents, as is known in the art. The size of a tumor mass can also be ascertained by physical means, such as palpation of the mass or measurement of the mass with a measuring instrument such as a caliper.

The invention will now be illustrated by the following non-limiting examples.

### EXAMPLES

Materials and methods used in Examples 1 and 2:

Case selection and processing of tissue for histological evaluation—The paraffin wax blocks from a total of 10 pretreatment surgical specimens of ocular retinoblastomas and three normal retina samples, obtained from a patient who underwent enucleation for painful absolute glaucoma, were collected at the Department of Human Pathology and Oncology, University of Siena, Italy. Tissues were cut and fixed in a buffered 4% aqueous formaldehyde solution, pH 7.4. For conventional histology, 4-μm-thick sections were obtained from representative paraffin wax blocks and stained with hemalum and eosin, Giemsa, periodic acid-Schiff (PAS), Gomori's silver impregnation and Feulgen.

Immunohistochemistry—Consecutive sections of retinoblastoma tissue cut at 3-μm thickness were subjected to immunostaining. Tissue samples of normal retina from three unrelated patients were used as the control. The EnVision™ +HRP method (Dako, Milan, Italy) was used to visualize immunohistochemical reaction products. Antigen retrieval was achieved by the treatment of deparaffinized sections with microwaves in 1 mM EDTA, pH 8.0, for 5 min, followed by cooling at room temperature prior to incubation with the antibodies. The monoclonal antibody anti-pRb2/p130 was obtained from Transduction Laboratories (Lexington, Ky., USA), and was used at a dilution of 1:100 in TBS. The primary antibodies were replaced with normal mouse serum to obtain negative controls. Normal human tonsils served as positive controls.

Laser capture microdissection and DNA extraction—Three normal human retina and seven retinoblastoma archived paraffin-fixed tissues were identified on hemotoxylin and eosin (H&E)-stained sections and isolated by laser capture micro-dissection (Arcturus PixCell II™ MWG-BIOTECH, Florence, Italy). The selected samples were adhered to a Capsure™ transfer film. The Capsure™ transfer film carrier was placed directly into a standard microcentrifuge tube containing digestion buffer (50 μl buffer containing 0.04% Proteinase K, 10 mM Tris-HCL (pH 8.0), 1 mM EDTA and 1% Tween-20). The tube was preheated upright in a 37° C. oven for 5 min. and then placed upside down so that the digestion buffer contacted the tissue on the cap. The samples were incubated overnight at 37° C., centrifuged for 5 min. and the cap was removed. The samples were heated to 95° C. for 8 min. to inactivate the Proteinase K and used directly as template for PCR reactions. Genomic DNAs were extracted from three frozen retinoblastoma samples, Weri-Rb1 cells and blood samples from 15 healthy donors according to the manufacturer's standard instructions.

RB2/p130 mutational analysis and methylation-specific PCR (MSP) assay—PCR of genomic DNA extracted from microdissected primary tumors and Weri-Rb1 cells (cultured as described below) was performed for mutational analysis of RB2/p130. All 22 exons were amplified (for list of primers, see Table 2) at an annealing temperature of 55° C. and sequenced. The methylation status of the 10 retinoblastoma specimens and Weri-Rb1 cells was examined in the CpG region immediately 5′ to the transcription started site (ATG) and inside Exon 1 and Intron 1. These regions were identified by using the CpG Ware™ primer design software (Intergen, Purchase, N.Y., USA). The assay is based on the DNA sequence differences between methylated and unmethylated DNA after bisulfite modification by DNA modification CpGenome Kit (Intergen, Purchase, N.Y., USA). The bisulfite reactions were performed according to the manufacturer's instructions. Subsequent PCR with primers specifically designed for discriminating between methylated (Tm=66° C.), unmethylated (Tm=61° C.) and wild-type DNA (Tm=68° C.) was performed (see Table 3 for list of primers). PCR products were analyzed on a 2.5% agarose gel.

TABLE 2
Primers for PCR analysis of RB2/p130 DNA

| | | | |
|---|---|---|---|
| Primer Name | Sequence | Size (bp) | SEQ ID NO: |

| | | | |
|---|---|---|---|
| Exon 1-F | 5′-CCT CAC CTC ACC TGA GGT-3′ | 329 | 10 |
| Exon 1-R | 5′-ACC GGT TCA CAC CAA CTA GG-3′ | | 11 |
| Exon 2-F | 5′-GAG ATA GGG TCA TCA TTG AAA C-3′ | 206 | 12 |
| Exon 2-R | 5′-CAT TAG CCA TAC TCT ACT TGT-3′ | | 13 |
| Exon 3-F | 5′-AGC TAG TCA GAG ACA TGA GTT G-3′ | 402 | 14 |
| Exon 3-R | 5′-CAC TGC AGC ACA GAC TAA TGT GT-3′ | | 15 |
| Exon 4-F | 5′-TCT CTC CCT TTA ACT GTG GGT TT-3′ | 245 | 16 |
| Exon 4-R | 5′-GGA GTT GAC GAG ATT AAT ACC TG-3′ | | 17 |
| Exon 5-F | 5′-CTC TGT AAC TGC TTA TAA TCC TG-3′ | 235 | 18 |
| Exon 5-R | 5′-CTA GGA AAC CTG TAC AAC TCC-3′ | | 19 |
| Exon 6-F | 5′-GGC TTA TTG TGT GCT GAT ATC-3′ | 298 | 20 |
| Exon 6-R | 5′-AGA GAT CCT TAA GTC GTC ATG-3′ | | 21 |
| Exon 7-F | 5′-CAT GAC GAC TTA AGG ATC TCT T-3′ | 196 | 22 |
| Exon 7-R | 5′-CTC AGT TTC CAG AGT ACA AAC-3′ | | 23 |
| Exon 8-F | 5′-CAG TTT CTG TGA GAG AGT ACA-3′ | 283 | 24 |
| Exon 8-R | 5′-GGC TTA CCT GCT CCT GGT ATT T-3′ | | 25 |
| Exon 9-F | 5′-GTG AAT TAA AGT CTT TCT GGG G-3′ | 244 | 26 |
| Exon 9-R | 5′-ATC TTA GAA AGC AGA CAG GGC-3′ | | 27 |
| Exon 10-F | 5′-GAG ACA TTT TAT CCC CTT GTG-3′ | 307 | 28 |
| Exon 10-R | 5′-TCC ATG CCT CCA GTC TAA AGT-3′ | | 29 |
| Exon 11-F | 5′-GAG GAG GAA TGG GCC TTT ATT-3′ | 244 | 30 |
| Exon 11-R | 5′-ACC CAC AGA ATA GGG CAG GA-3′ | | 31 |
| Exon 12-F | 5′-CAC TTA AGT TGC ACT GGG TA-3′ | 273 | 32 |
| Exon 12-R | 5′-CAA CAG GAA GTT GGT CTC ATC-3′ | | 33 |
| Exon 13-F | 5′-TAA AAG GAA GAG CGG CTG TTT-3′ | 378 | 34 |
| Exon 13-R | 5′-TTA AAC CTA ACT GCC ACC CTC-3′ | | 35 |
| Exon 14-F | 5′-GGA TAC TGG CAT TCT GTG TAA C-3′ | 197 | 36 |
| Exon 14-R | 5′-ATT TCC AGA TAG TAA GCC CCA-3′ | | 37 |
| Exon 15-F | 5′-AGC TTG GAC GGA AGT CAG ATC-3′ | 413 | 38 |
| Exon 15-R | 5′-TCT AGC CAA ACC TCG GGT AAC-3′ | | 39 |
| Exon 16-F | 5′-AAT TGT AAA CCT CTG CCC-3′ | 392 | 40 |
| Exon 16-R | 5′-ATT TCC CAA GCT CAT GCT-3′ | | 41 |
| Exon 17-F | 5′-AGC ATG AGC TTG GGA AAT-3′ | 275 | 42 |
| Exon 17-R | 5′-TGA AGA CCT ATC TTT GCC-3′ | | 43 |
| Exon 18-F | 5′-GTT CAC AGA GCT CCT CAC ACT-3′ | 230 | 44 |
| Exon 18-R | 5′-AGG CCA CAG AGT CAA CTA TGG-3′ | | 45 |
| Exon 19-F | 5′-AGG TCC TAT CAC CAA GGG TGT-3′ | 250 | 46 |
| Exon 19-R | 5′-GCT TAG TTA CTT CTT CAA GGC-3′ | | 47 |
| Exon 20-F | 5′-GAG AAA GTT AAT ATC CTA GCT G-3′ | 446 | 48 |
| Exon 20-R | 5′-GTG AAT GGT CCA TAT ATA AAT CA-3′ | | 49 |
| Exon 21-F | 5′-TGG TTT AGC ACA CCT CTT CAC-3′ | 325 | 50 |
| Exon 21-R | 5′-GCT TAG CAC AAA CCC TGT TTC-3′ | | 51 |
| Exon 22-F | 5′-CTG AGC TAT GTG CAT TTG CA-3′ | 232 | 52 |
| Exon 22-R | 5′-AAG GCT GCT GCT AAA CAG AT-3′ | | 53 |

Sequences of forward (F) and reverse (R) primers used for PCR analysis of RB2/p130 gene exons. The size of the PCR product amplified by a given primer pair is indicated in the third column.

TABLE 3
Primers for MSP analysis of RB2/p130 CpG-enriched regions

| | | | |
|---|---|---|---|
| Region | Primer Name | Sequence | SEQ ID NO: |

| | | | |
|---|---|---|---|
| 1 Unmethylated- | Rb2/U1-F | 5′-AAC ACA ATA CAA ACA ACA AAC AAA CAA AGA-3′ | 54 |
| 100 to +177 bp | Rb2/U1-R | 5′-GTT GTT TTA GGT TTT GGT TTG TGT TGT TTT-3′ | 55 |
| 1 Methylated- | Rb2/M1-F | 5′-GAT ACG AAC GAC GAA CGA ACG AAC G-3′ | 56 |
| 95 to +177 bp | Rb2/M1-R | 5′-TTT TAG GTT TCG GTT CGC GTC GTT TC-3′ | 57 |
| 2 Unmethylated | Rb2/U2-F | 5′-CCT CAA CAT AAA CAA AAC AAC ACA AAC CA-3′ | 58 |
| +164 to +302 bp | Rb2/U2-F | 5′-TTT GAG AGT TTT TTG AGG TGT GTG ATG T-3′ | 59 |
| 2 Methylated | Rb2/M2-F | 5′-CAA CAT AAA CGA AAC GAC GCG AAC CG-3′ | 60 |
| +167 to +302 bp | Rb2/M2-R | 5′-GAG TTT TTC GAG GCG CGC GAC GC-3′ | 61 |
| 3 Unmethylated | Rb2/U3-F | 5′-GAA TTG GTG TTT TTT GAG TTG TGT TGT GT-3′ | 62 |
| +282 to +411 bp | Rb2/U3-R | 5′-AAA AAC CAC AAA AAA ACA CAA CAA AAA CAA A-3′ | 63 |
| 3 Methylated | Rb2/M3-F | 5′-GGT GTT CGA GTC GCG TCG CGC-3′ | 64 |
| +287 to +411 bp | Rb2/M3-F | 5′-CCG CAA AAA AAC GCG ACG AAA ACG AA-3′ | 65 |
| Wild-type | Rb2/WT-F | 5′-GCC TTC CGA GCC GCG TCG CGC-3′ | 66 |
| +290 to +411 bp | Rb2/WT-R | 5′-AGG AAA GCG CGG CGA GAG CGG G-3′ | 67 |

Sequences of forward (F) and reverse (R) primers used for MSP assay. Their positions on RB2/p130 genomic DNA are indicated (0 bp indicates the first base pair of the transcription start site, ATG).

Weri-Rb1 cell line and 5-Aza-2-dc DNA methyltransferase inhibitor treatment—Human retinoblastoma cell line (Weri-Rb1 ) obtained from the American Type Culture Collection (ATCC, Manassas, Va., USA) was cultured in RPMI 1640 supplemented with 10% FCS at spin ratio of 1:2 once a week. A volume of 2.5 μm of 5-Aza-2-dc, a DNA methyltransferase inhibitor (Sigma-Aldrich, St. Louis, Mo., USA) was added to medium culture of treated cells.

Cell viability (MTT) and FACS analysis—Quantitative cell viability was measured by colorimetric assay using a cell proliferation kit (MTT) (Roche Molecular Biochemicals, Mannheim, Germany). A total of 5000 cells/well Weri-Rb1 and 5-Aza-2-dc-treated Weri-Rb1 were grown in microtiter plates (96-well) in a final volume of 100 μl culture medium. The incubation period of cell culture was 24, 48 and 96 h in the presence or absence of the 2.5 μM DNA methyltransferase inhibitor. After incubation period, 10 μl MTT labeling reagent was added to each well to a final concentration 0.5 μg/ml. MTT is cleaved by growing cells to form formazan crystals which allows quantification of cell viability by spectrdphotometric analysis (ELISA) at 550 nm. Cell viability was expressed as the percentage of the absorbance of drug-treated and untreated cells relative to that of the untreated cells of 0 h. FACS analysis was carried out on cells treated with 5-Aza-2-dc and compared to the untreated (control) cells after 24, 36, 28, 72 and 96 h in culture.

Western blotting—Western blot analysis of pRb/p105 and pRb2/p130 was performed on three fresh samples of primary tumors (samples 8-10). Weri-Rb1 cells and one normal retina sample, obtained from a patient who underwent enucleation for painful absolute glaucoma, served as an experimental control. Fresh tissues were immediately frozen in liquid nitrogen. Furthermore, Weri-Rb1 cells treated and untreated with 5-Aza-2-dc at various times were processed for Western blot analysis. Whole tissues lysates were prepared by resuspending homogenized tissues and pelleted cells in lysis buffer (50 mM Tris/HCl, 5 mM EDTA, 250 mM NaCl, 50 mM NaF, 0.1% Triton X-100, 0.1 mM Na₃VO₄ plus fresh inhibitors). Equal amounts of 100 μg of total extracts were loaded and resolved on a 7 or 10% SDS-PAGE. The gels were then transferred onto a nitrocellulose filter and checked by using 0.1% Ponceau red. The polyclonal anti-pRb/p105 (C15) (Santa Cruz, Santa Cruz, Calif., USA) and monoclonal anti-pRb2/p130 (Transduction Laboratories, Lexington, Ky., USA) were used at a dilution of 1:300 and 1:500, respectively. The monoclonal anti-p53 (Ab6) (Calbiochem, Cambridge, Mass., USA) was used at a dilution of 1:1000, while the monoclonal anti-E2F1 (KH95), polyclonal anti-E2F4 (C20) and polyclonal anti-p73 (H79) (Santa Cruz, Santa Cruz, Calif., USA) were used at dilution of 1:200. The anti-actin antibody (Santa Cruz, Santa Cruz, Calif., USA) was used as loading control following the manufacturer's instructions.

### Example 1

### Significance of pRb2/p130 Expression Level with Respect to Mutational Status

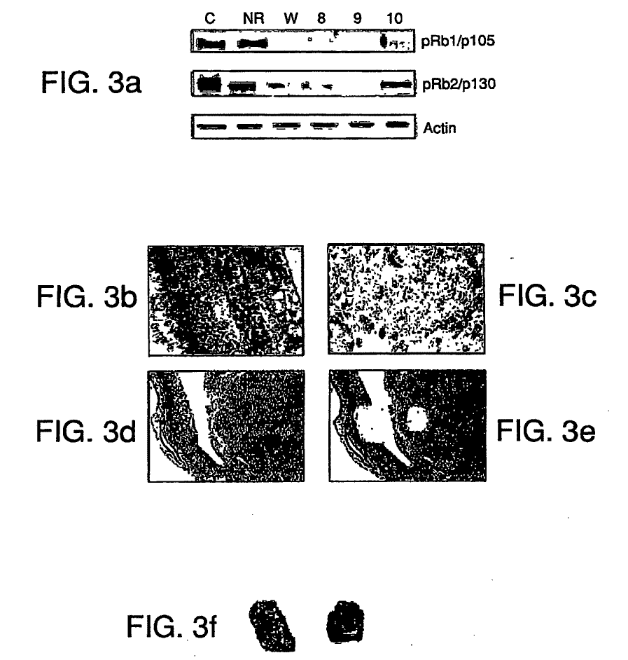

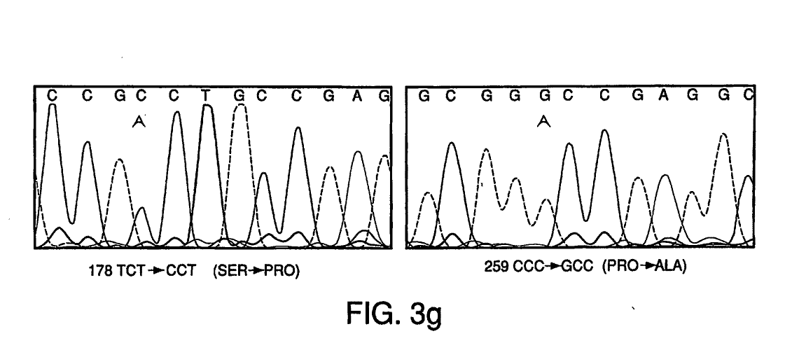

Experiments were performed on 10 retinoblastoma primary tumors (two familial and eight sporadic), Weri-Rb1 cells and three normal retina samples. The pRb1/p105 and pRb2/p130 protein expression levels were evaluated by Western blot analysis in normal retina (NR), in three frozen sporadic retinoblastoma samples (samples 8-10) and in Weri-Rb1 cells (W). Data are shown in FIG. 3a where the lack of pRb1/p105 expression and the downregulation of pRb2/p130 in both primary tumors and Weri-Rb1 cells is evident pRb2/p130 downregulation was confirmed on sections from paraffin-embedded tissues by immunohistochemical analysis, which has been extended to seven more retinoblastoma cases (FIGS. 3b and 3c). This further analysis evidenced differences in pRb2/p130 expression level among various patients (see Table 1 above). In particular, downregulation was detected in four out of 10 samples, while in four cases pRb2/p130 was not expressed and in the remaining two cases there was no difference with respect to normal retina. The only correlation between this result and clinicopathological classification was that both samples which were indistinguishable from normal retina arose from bilateral familial retinoblastoma (B/F) patients.

To check whether differences in pRb2/p130 expression level depend on different mutational patterns, DNA samples were screened for mutations in RB2/p130 coding regions using properly designed primers able to amplify each of the 22 RB2/p130 exons (Table 2 above). Tissue from normal retinas and tumor specimens were isolated by laser capture microdissection (FIG. 3d-f) and DNA was extracted and processed by PCR. The PCR product sequences were matched with the wild-type RB2/p130 sequences (see Gene Bank Accession Numbers X74594 and U53220 and Mayol et al., 1993; Baldi et al., 1996, the entire disclosures of which are herein incorporated by reference). With the exception of one familial retinoblastoma patient, two Exon 1 mutations were detected at nucleotides 178 and 259 (TCT→CCT: SER→PRO and CCC→GCC: PRO→ALA) in all primary tumors (Table 1, FIG. 3g). Exon 1 mutations were not detected either in normal retina samples or in 15 healthy donors' blood samples (Table 1). In primary tumors, a clear correlation was found between RB2/p130 expression level and exon 1 homozygous/heterozygous mutations. In particular, loss of expression is correlated with double homozygous mutation (Table 1, samples 3-5, 9), and weak expression coincides with the presence of nucleotide 178 homozygous and nucleotide 259 heterozygous mutations (Table 1, samples 6-8, 10), while when both mutations are heterozygous, the expression level is normal (Table 1, sample 2). Screening of RB2/p130 mutational pattern in the Weri-Rb1 retinoblastoma cell line evidenced the same Exon 1 homozygous mutations observed in nine out of 10 primary tumors.

Further mutations were detected in both primary tumors and the Weri-Rb1 retinoblastoma cell line in RB2/p130 exons 4, 6, 13, 16 and 21 (see Table 1 for details). Three additional homozygous silent mutations in Exons 15 and 17 were present in both control (normal retina and healthy donors) and retinoblastoma samples (primary tumors and Weri-Rb1 cells). A further homozygous silent mutation in Exon 12 was detectable only in sporadic retinoblastoma tumors (six out of eight samples; see Table 1). These results suggest that the two silent mutations in Exons 15 and 17 represent a gene polymorphism occurring naturally in the population, while the homozygous silent mutation in Exon 12 is specific for sporadic retinoblastoma and is therefore predictive with respect to tumor phenotype.

### Example 2

### Role of Epigenetic Events on RB2/p130 Gene Expression

CpG islands, which are potential methylation sites, are often found near the promoters of widely expressed genes and typically extend into the first exon. CpG islands can also occur downstream from transcription start sites and are unmethylated in normal cells, although such islands seem to be preferential targets for de novo methylation in human cancer.

Therefore, all samples from Example 1 were processed by methylation specific—PCR (MSP) assay, focusing the analysis on three RB2/p130 regions rich in CpG dinucleotide: the promoter region immediately 5′-flaking to the transcription start site (ATG) from about nucleotide −95 to about +177 (“Region 1”), the region is from about nucleotide +167 to about +302, encompassing most of exon 1 (“Region 2”), and the region is from about nucleotide +287 to about +411, encompassing the 3′-end of exon 1 and the 5′-end of intron 1 (“Region 3”) (see FIG. 2a and Table 3). In FIG. 2b, the results obtained from three samples chosen as representative of pRb2/p130 expression level pattern (samples 2, 3 and 8 from Table 1) are reported. As shown in FIG. 2b, when pRb2/p130 expression level is normal (+ + +), all the three examined regions were unmethylated (U1, U2 and U3). Loss of expression (−) corresponded to methylation of the Regions 1, 2 and 3 (M1, M2 and M3), while downregulation (+) was correlated to methylation of Region 3 alone (Table 1). These results suggest that Exon 1 mutation pattern could establish the susceptibility to gene methylation which, in turn, would determine the protein expression level.

To verify whether removal of the transcriptional block due to RB2/p130 methylation alters the expression level of endogenous pRb2/p130 and restores its tumor-suppressor function, Weri-Rb1 cells were treated with the DNA methyltransferase inhibitor 5-Aza-2-deoxycytidine (5-Aza-dC). Data obtained in Weri-Rb1 cells indicated that, in this model, the percentage of methylation and therefore of RB2/p130 expression level were not as correlated with mutational status as had been found for primary tumors. However, the effects of demethylating agents could be adequately investigated in this, the sole experimental model available.

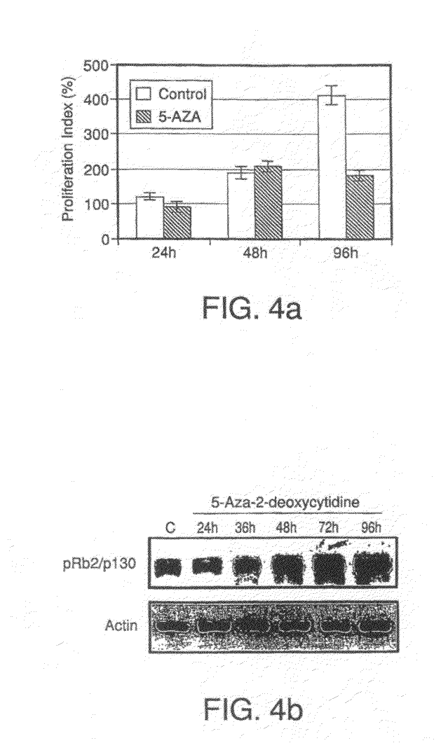

The effect of the demethylating agent on Weri-Rb1 cell proliferation as a function of treatment duration is shown in FIG. 4a. As is evident from the figure, a significant difference in proliferation rate was detectable after 96 h of treatment when the number of cells was markedly reduced. FACS analysis revealed that this reduction in total cell number corresponded to an increase in the amount of apoptotic cells (20% of treated cells vs. 6% of control). Moreover, preponderance of GI arrested cells was already detected after 24 (40% of treated cells vs. 30% of control). Western blot analysis showed that the effects of the de-methylating treatment on cell growth were concomitant with increased expression of endogenous pRb2/p130 with respect to the control obtained after 96 h cell culture (FIG. 4b).

The following materials and methods were used in Examples 3 and 4:

Cell cultures and treatment—Cell lines were purchased from the American Type Culture Collection (Rockville, Md.). The cells were cultured in DMEM or RPMI1640 medium supplemented with 10% fetal bovine serum and 2 mM L-glutamine. For treatment, cells were seeded at a density of 5×10⁵ cells/100-mm tissue culture dish. 2.5 μM of DNA methyltransferase inhibitor (5-AZA-2-deoxicytidine) was added to the culture medium for up 96 hours.

Analysis of RB2/p130 mRNA and protein expression level in H23 cells—Total RNA from the human non-small cell lung cancer (H23) cell line was extracted using TRIzol (Life Technologies) according to the manufacturer's protocol. The RNA was electrophoresed in a formaldehyde (Sigma) agarose gel (Kodak), transferred overnight to a Hybond N⁺ Nylon membrane (Amersham) and the filter was UW cross-linked. The membrane was hybridized with a random primer labeled cDNA probe (RB2/p130 fragment), washed and exposed to a Kodak X-ray film at −80° C. The levels of RB2/p130 mRNA were normalized with the level of GAPDH mRNA.

Western blot analysis of pRb2/p130 was performed using total protein lysates from untreated and 5-AZA-2-deoxicytidine treated H23 cells. Whole cell lysates were prepared in lysis buffer (50 mM Tris/HCl, 5 mM EDTA, 250 mM NaCl, 50 mM NaF, 0.1% Triton-X, 0.1 mM Na₃VO₄ plus fresh inhibitors). Equal amounts of 100 μg of total extracts were loaded and resolved on a 7% SDS-PAGE. The gels were then transferred onto a nitrocellulose filter and checked by using 0.1% Ponceau red. The monoclonal anti-pRb2/p130 (Transduction Laboratories, Ky., USA) was used at a dilution of 1:800.

RB2/p130 Mutational analysis and Methylation-specific PCR (MSP) assay—High molecular weight genomic DNA from H23 cells was obtained using the Qiamp Tissue Kit (Qiagen, Valencia, Calif.) following the manufacturer's suggested protocol. PCR of genomic DNA was used to perform mutational analysis of RB2/p130. All 22 exons were amplified by PCR (see Table 2 above for primer sequences) and sequenced as in Example 1 above.

The methylation status of Regions 1, 2 and 3 RB2/p130 was evaluated with a methylation specific PCR (MSP) assay (CpGenome, Intergene, N.Y., USA) as described above. Primers were specifically designed to discriminate among methylated (Tm=66° C.), unmethylated (Tm=61° C.) and wild-type DNA (Tm=68° C.) (FIG. 3a-b). PCR products were analyzed on 2.5% agarose gel (see Table 3 above).

Multiplex RT-PCR—Multiplex RT-PCR analysis was carried out with 0.2 μg of total RNA from treated and untreated H23 cells. PCR was performed using RB2/p130 primers and β-actin specific primers (SuperScript RT-PCR System, Invitrogen).

### Example 3

### Mutational Analysis of RB2/p130

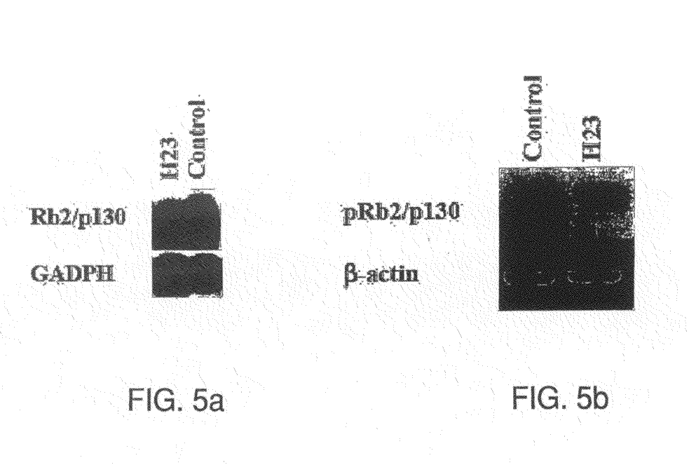

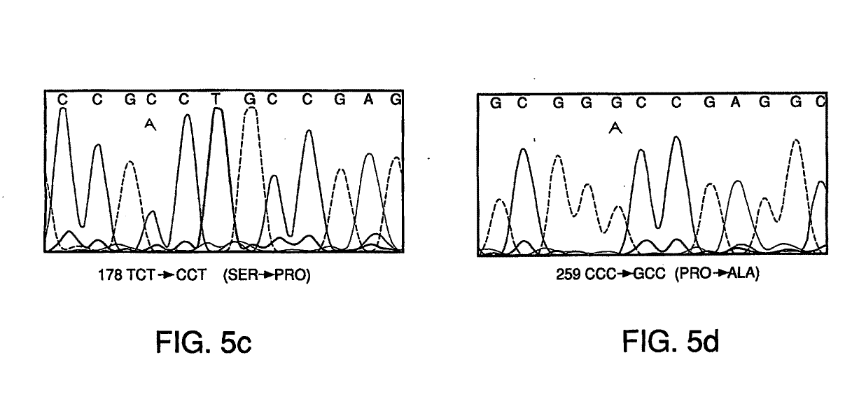

To investigate if the low expression levels of both RB2/p130 mRNA and protein in non-small lung carcinoma H23 cell line (FIG. 5a-b) is due to genetic alterations occurring in Rb2/p130 gene, H23 DNA was screened for mutations in RB2/p130 coding regions by using properly designed primers able to amplify each of the 22 exons, as described in Example 1 above. Two exon 1 homozygous mutations at nucleotides 178 and 259 were found in H23 cells (FIG. 5c-d). To confirm that these mutation are associated with a tumoral phenotype, the nucleotide sequence of RB2/p130 exon 1 was screened in the following cancer cell lines and primary tumors: T-lymphoblastoid leukemia (CCRF-CEM, Molt-1 and Jurkat), B-lymphoblastoid leukemia (Daudi), chronic myeloid leukemia (K562), breast carcinoma (SK-Br3 and MCF-7), retinoblastoma (Weri-Rb1) and colon cancer (HT29) cell lines; and in retinoblastoma, ovarian, colon and endometrial primary tumors. In all these samples, exon 1 homozygous mutations were detected at nucleotides 178 and 259 (FIG. 5c-d)

The exon 1 homozygous mutations at nucleotides 178 and 259 were also present in normal-appearing endometrial tissue (as confirmed by histology) derived from the same subjects taken in an adjacent non-tumoral area as far as possible from the tumor site. Moreover, no mutations were found from exon 2 to 22 in H23 cells. In addition, no mutations were detected in normal retina, lung, ovary, endometrium, breast and colon tissues from patients affected by non-tumoral pathologies and in blood samples from 15 healthy donors.

### Example 4

### Methylation Status of RB2/p130 in H23 Cells and Restoration of RB2/p130 Expression

The methylation status of RB2/p130 gene in H23 cells was examined as described in Example 2 above. By performing Methylation Specific-PCR (MSP) assay, the methylation of RB2/p130 Region 1 (−95 bp to +177 bp) and Region 3 (+287 bp to +411 bp) in H23 cells (FIG. 6).

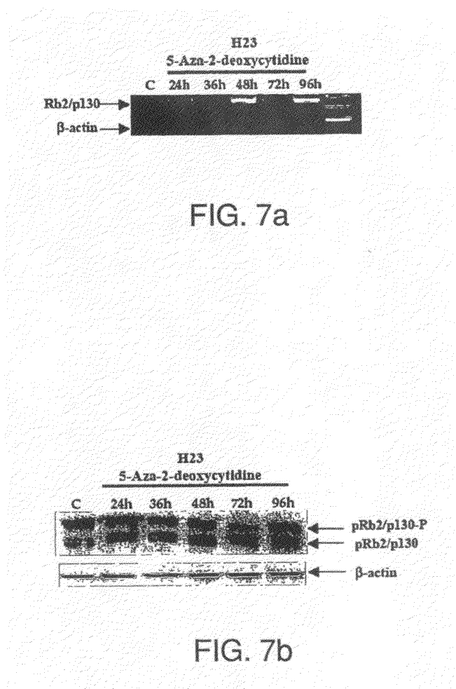

The effect of the DNA methyltransferase inhibitor 5-AZA-2-deoxycytidine on RB2/p130 expression was also assessed, including whether the 5-Aza-dC-treatment was sufficient to restore the expression of endogenous pRb2/p130 in H23 cells. The effect of the 5-Aza-dC on both RB2/p130 mRNA and protein expression level at different time points post-treatment is shown in FIGS. 7a and 7b. By using multiplex RT/PCR, an increase of RB2/p130 mRNA level was observed, beginning at 48 hours after the 5-Aza-dC-treatment (FIG. 7a). Moreover, an increase of pRb2/p130 protein levels, in its active hypophosphorylated form, was observed at 72 hours post-5-Aza-dC treatment, reaching at maximum at 96 hours (FIG. 7b). These data indicate that epigenetic events can occur to down-regulate RB2/p130 transcription in non-small lung cancer H23 cells.

Materials and methods used for Examples 5 through 9:

Tissue procurement and cell culture—Twelve couples of paired normal human cornea and conjunctiva biopsies were obtained from the Delaware Valley Lions Eye Bank, from patients undergoing routine cataract surgery. Informed consent was obtained from these patients in accordance with the regulations of the Institutional Review Board of the University of Pennsylvania. Primary cornea and conjunctiva cell lines were initiated from the biopsies as previously described in Williams et al, 1999, Investigative Ophthalmology & Visual Science 40 (8): 1669-1675, the entire disclosure of which is herein incorporated by reference. Cells between the first and sixth passages were used for the experiments.

Multiplex RT-PCR—Total RNA was extracted from paired normal human primary cornea and conjunctiva cells by using the RNeasy kit (Qiagen) according to the manufacturer's instructions. Before further use, the extracted RNA was treated with DNase I, amplification grade (1 U DNase/1 mg total RNA; Life Technologies). Reverse transcription—polymerase chain reaction (“RT-PCR”) was performed by using the Reverse Transcription System (Promega). Multiplex RT-PCR was carried out using 1/100 of cDNA and the following primers for each reaction: PAI-2: 5′-tgacaaactcaacaagtgga-3′ (forward; SEQ ID NO: 68), 5′-tgcataagataaccaactgc-3′ (reverse; SEQ ID NO: 69); β-actin: 5′-tgacgggctcacccacactgtgccca-3′ (forward; SEQ ID NO: 70), 5′-ctagaagcatttgcggtggacgatgg-3′ (reverse; SEQ ID NO: 71). 0.3:2.0 was the primer ratio for β-actin and PAI-2, respectively. The amplified fragments were detected by 1.5% (w/w) agarose gel electrophoresis. Each band was quantified and the specific gene expression level was determined semi-quantitatively by calculating the ratio of densitometric value from the PAI-2 band in relation to the internal standard represented by β-actin.

Western blot and chromatin immuoprecipitation assay—Cytoplasmic and nuclear proteins were extracted from 12 couples of paired normal human corneal and conjunctival epithelial cells by using the PARIS kit (Ambion) according to the manufacturer's instructions. Efficient cytoplasmic and nuclear fractionation was confirmed by Western blotting analysis using anti-GAPDH antibody for the cytoplasmic fraction and anti Oct-1 antibody for the nuclear fraction. Immunoprecipitation experiments were performed using the cytoplasmic and nuclear fractions with PAI-2 as immunoprecipitating antibody (N-18, Santa Cruz Biotechnology, Calif.). The presence of pRb2/p130, Rb1/p105 and p107 in both PAI-2 nuclear and cytoplasmic precipitates was assessed with anti-pRb2/p130, anti-Rb1/p105 and p107 antibodies (211.6, C-15 and C-18, respectively; Santa Cruz Biotechnology, Calif.) by Western blotting.

Cross-linked chromatin immunoprecipitation (“XchIP”)—XChIPs were performed as previously described in Macaluso et al, 2003, Oncogene 22(42): 6472-6478, the entire disclosure of which is herein incorporated by reference. Cornea and conjunctiva cells were cross-linked by adding formaldehyde (1% final concentration) directly to culture medium and incubating at 37° C. Immunoprecipitations were carried out using 3-4 μg of antibodies against pRb2/p130, Rb1/p105, p107, E2F1, E2F4, E2F5, DNMT1, p300, PAI-2 (Santa Cruz Biotechnology), HDAC1, SUV39H1 (Upstate Biotechnology) or ICBP90. As negative controls; both no-antibody immunoprecipitations and immunoprecipitations with an irrelevant antibody were performed. The cross-link was reversed by incubating samples at 65° C. overnight, and DNA was extracted with phenol:chloroform and precipitated with ethanol. Primers spanning a specific region of PAI-2 promoter were used in the PCR reactions (P1: 5′-atacccgaagaaaattagga-3′ (forward; SEQ ID NO: 72); P2: 5′-aagttgcagttctaacgtaga-3′ (reverse; SEQ ID NO: 73); see GenBank accession no. M22469, the entire disclosure of which is herein incorporated by reference). 1% of total chromatin (“Input”) was used as positive control.

### Example 5

### Protein Expression Levels of ICBP90 in MCF-7, MDA-MB-231 and MDA-MB-361

Breast cancer cell lines MCF-7, MDA-MB-231 and MDA-MB-361 were obtained from the ATCC and cultured using standard growth conditions and media. Total cell lysates were obtained as described above for the corneal and conjuntival cells, and the lysates were subjected to Western blot analysis as described previously, using anti-ICBP90 antibody. β-actin was detected with an anti-β-actin antibody as a loading control. The results are presented in FIG. 8.

### Example 6

### Interaction between pRb2/p130 and ICBP90 in Nuclear and Cytoplasmic Fractions of MCF-7, MDA-MB-231 and MDA-MDA-361 Cells

Total cell lysates from MCF-7, MDA-MB-231 and MDA-MB-361 cells were obtained as in Example 5 and were separated into nuclear and cytoplasmic fractions. Each fraction was immunoprecipitated with anti-pRb2/p130 antibody, and the immunoprecipitate was subjected to Western blot analysis using anti-ICBP90 antibody. As shown in FIG. 9, ICBP90 is associated with pRb2/p130 in the nuclear fractions of cultured MCF-7, MDA-MB-231 and MDA-MB-361 cells. However, ICBP90 is associated with pRb2/p130 only the cytoplasmic fraction of cultured MCF-7 cells.

### Example 7

### In Vivo Binding of ICBP90 to the ER-α Promoter in MDA-MB-231 Cells

MDA-MB-231 cells were cross-linked with formaldehyde for XChIP analysis, as described above. Primers flanking estrogen receptor (“ER”)-α promoter regions 1 and 2 as indicated in FIG. 10a were used to amplify chromatin immunoprecipitated with anti-ICBP90 antibody. As shown in FIG. 10b, ICBP90 binds to estrogen receptor α promoter region 1 but not to region 2.

Without wishing to be bound by any theory, the results shown in FIGS. 8, 9 and 10 indicate that pRb2/p130 could control the biochemical balance between cytoplasmic and nuclear ICBP90, and that ICBP90 and pRb2/p130 could be involved in a common mechanism controlling gene transcription. For example, in MCF-7 cells, pRb2/p130 could retain ICBP90 in the cytoplasm, lowering the concentration of this protein in the nucleus and thus limiting its function. In MDA-MB-231 cells, higher concentration of ICBP90 in the nucleus should permit the binding of ICBP90 to a specific methylated sites in the estrogen receptors promoter. Therefore, the interaction between ICBP90 and pRb2/p130 could be responsible for the recruitment of DNMT1 to the pRb2/p130-complex in MDA-MB-231, and for the recruitment of DNMT1 around the ER-α promoter region. ICBP90 could thus be the E3 ligase that ubiquitinates H3, which could be one of the first steps of chromatin remodeling, allowing subsequent recruitment of DNMT1 on the ER-α promoter. The resultant histone deacetylation and methylation, and DNA methylation could create a heritable mark to establish a heterochromatin state of long-term silencing.

### Example 8

### Anti-PAI-2 Antibody Co-Immunoprecipitates pRb2/p130 and Rb1/p105, but not p107, in the Cytoplasm and Nucleus of Normal Primary Human Corneal and Conjunctival Epithelial Cells

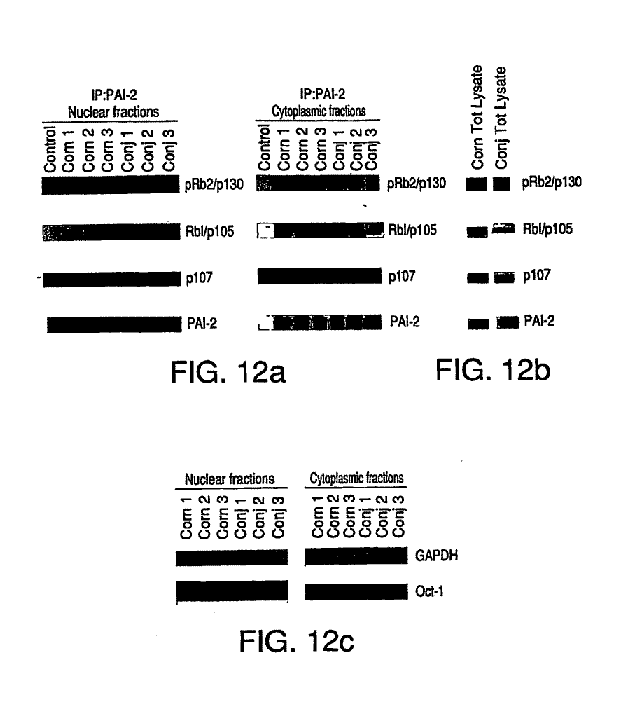

In order to assess whether PAI-2 associated with the retinoblastoma family proteins, PAI-2 was immunoprecipitated from nuclear and cytoplasmic lysates of twelve paired couples of normal primary human corneal and conjunctival epithelial cells. The immunoprecipitates were then analyzed by Western blotting using anti-pRb2/p130, anti-Rb1/p105, anti-p107 and anti-PAI-2 antibodies. For all the cell lines analyzed, anti-PAI-2 antibody co-immunoprecitated pRb2/p130 and Rb1/p105 from both nuclear and cytoplasmic fractions. On the contrary, no binding was detected between PAI-2 and p107 (FIG. 12a). Most of the pRb2/p130 immunoprecipitated by anti-PAI-2 antibody from both cornea and conjunctiva cytoplasmic fractions exhibited a hyperphosphorylated form (FIG. 12a, upper band), while in the corresponding nuclear fractions, most of pRb2/p130 exhibited an hypophosphorylated form (FIG. 12a, lower band). Moreover, as expected, the anti-PAI-2 antibody immunoprecipitated PAI-2 from both nuclear and cytoplasmic fractions of all the cell lines (FIG. 12a).

Western blotting analyses using total cell lysate displayed that both cornea and conjunctiva cells exhibit similar levels of pRb2/p130, Rb1/p105, p107 and PAI-2 proteins (FIG. 12b). The purity of the nuclear and cytoplasmic fractions was validated by using anti-GAPDH and anti-Oct1 antibodies, as cytoplasmic and nuclear markers, respectively (FIG. 12c). These data indicate a cytoplasmic and nuclear interaction between PAI-2 and specific members of pRb family proteins, pRb2/p130 and Rb1/p105, in normal human corneal and conjunctival epithelial cells.

### Example 9

### pRb2/p130, E2F5, HDAC1, DNMT1, SUV39H1 and PAI-2 Bind to the PAI-2 Proximal Promoter Region in Vivo

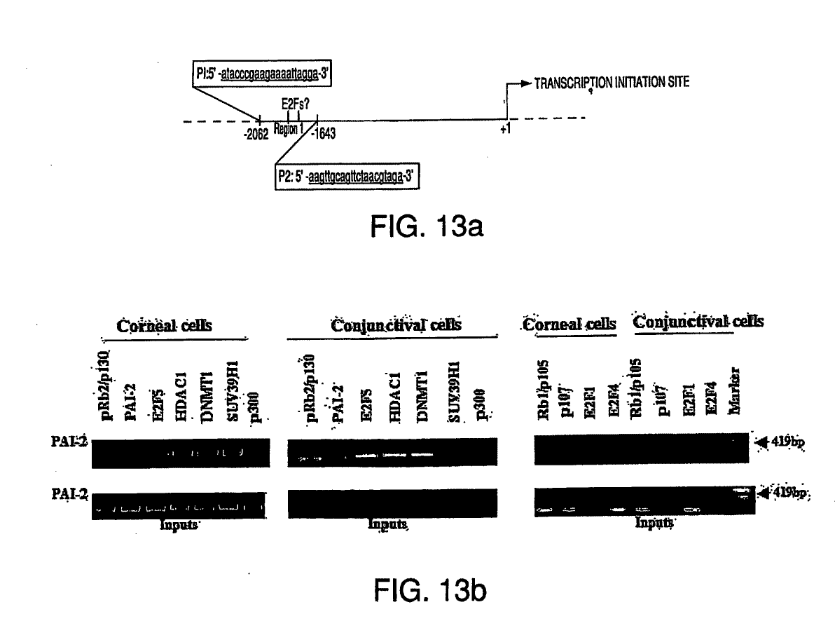

A specific PAI-2 promoter fragment between the residues −2062 and −1643 defines a negative regulatory element that represses PAI-2 promoter activity in a cell type independent manner and contains putative E2F binding sites (FIG. 13a); see Ogbourne et al., 2001, Nucleic Acids Res. 29 (19): 3919-3927, the entire disclosure of which is herein incorporated by reference. This information, along with the results shown in FIG. 12a, suggested the hypothesis that the pRb family proteins may be recruited on the PAI-2 promoter via binding of E2F factors, and that this interaction could have a physiological significance in controlling PAI-2 transcription, perhaps by chromatin remodeling. To investigate this, XChIP experiments were performed on cornea and conjunctiva cells using anti-pRb2/p130, anti-pRb1/p105, anti-E2F4, anti-E2F5, anti-E2F1, anti-HDAC1, anti-SUV39H1, anti-p300, anti-DNMT1 or anti-PAI-2 as immunoprecipitating antibodies. As shown in FIG. 13b, the XChIPs experiments indicate that pRb2/p130, E2F5, HDAC1, DNMT1, and PAI-2 bind in vivo to, simultaneously, a specific fragment of PAI-2 promoter in both normal primary human corneal and conjunctival epithelial cells. Moreover, SUV39H1 bound to the same PAI-2 promoter fragment only in cornea cells, while pRb1/p105, p107, E2F4, E2F1 and p300 were undetectable in both cornea and conjunctiva cells.

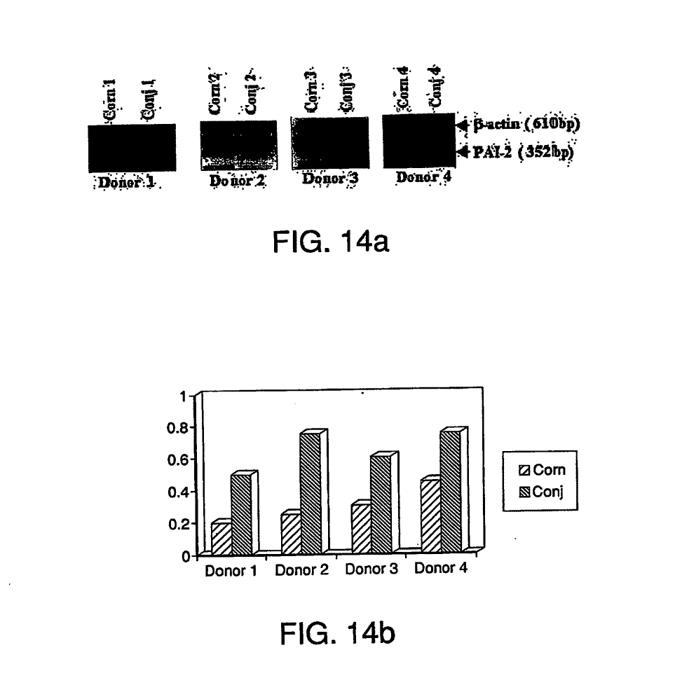

The results from a multiplex semiquantitative reverse transcription-polymerase chain reaction (RT-PCR) given in FIG. 14 show that the level of PAI-2 mRNA was higher in conjunctival cells than in corneal cells. These data indicate that corneal and conjunctival cell lines exhibit a specific PAI-2 gene expression pattern. Taken together, these data suggest that, in normal human corneal and conjunctival epithelial cells, the binding of pRb2/p130-PAI-2 complexes to a specific region of PAI-2 promoter may modulate the PAI-2 basal transcription by inducing local changes in chromatin structure. Under specific stimuli or at specific times of the cell cycle, the interaction of PAI-2 with pRb2/p130 and Rb1/p105 could permit the shuttle of PAI-2 between cytoplasm and nucleus, thus controlling the concentration of PAI-2 in these cellular compartments. Transcription of PAI-2 gene may be controlled by a feedback trigger loop from a specific PAI-2 concentration in the nucleus, which governs the binding of specific pRb2/p130-PAI-2-chromatin modifying complexes on the PAI-2 promoter. The interaction with E2F5 could be the primary mechanism by which pRb2/p130 is recruited to the chromatin regardless of the cell cycle stage, since recruitment on the chromatin of pRb2/p130 could function to alter the activity of transcription regulators bound nearby.

All documents referred to herein are incorporated by reference. While the present invention has been described in connection with the preferred embodiments and the various figures, it is to be understood that other similar embodiments may be used or modifications and additions made to the described embodiments for performing the same function of the present invention without deviating therefrom. Therefore, the present invention should not be limited to any single embodiment, but rather should be construed in breadth and scope in accordance with the recitation of the appended claims.

We claim:

1. A method of detecting tumor cells, comprising:
(1) obtaining a biological sample comprising test cells from a subject;
(2) obtaining nucleic acid from the test cells; and
(3) (i) analyzing the nucleic acid for mutations in exon 1 of the RB2/p130 gene, wherein the presence of homozygous mutations at nucleotides 178 or 259 of the RB2/p130 gene indicate that the test cells are tumor cells; or
(ii) when the nucleic acid obtained from the test cells comprises DNA, analyzing the methylation status of the RB2/p130 gene, wherein methylation of at least the region from about nucleotide +287 to about +411 of the RB2/p130 gene indicates that the test cells are tumor cells.

2. The method of claim 1, wherein the nucleic acid obtained from the test cells comprises DNA.

3. The method of claim 1, wherein the nucleic acid obtained from the test cells comprises RNA.

4. The method of claim 1, wherein the nucleic acid is obtained from the test cells is DNA and is analyzed for the methylation status of the RB2/p130 gene, and wherein the region from about nucleotide −95 to about +177 and the region from about nucleotide +167 to about +302 is also methylated.

5. The method of claim 1, wherein the tumor cells are from tumors derived from sarcomas, carcinomas, adenocarcinomas, cancers of neural origin, or hematological neoplasias.

6. The method of claim 1, wherein the tumor cells are from cancers having their origin in the breast; tissues of the male and female urogenital system; lung; tissues of the gastrointestinal system; exocrine glands; tissues of the mouth and esophagus; brain and spinal cord; kidney; pancreas; hepatobiliary system; lymphatic system; smooth and striated muscle; bone and bone marrow; skin; and tissues of the eye.

7. The method of claim 1, wherein the tumor cells are from retinoblastoma, lung cancer, T-lymphoblastoid leukemia, B-lymphoblastoid leukemia, chronic myeloid leukemia, breast carcinoma, colon cancer, ovarian cancer or endometrial cancer.

8. A method of diagnosing cancer, comprising:
(1) obtaining a biological sample comprising test cells from a subject;
(2) obtaining nucleic acid from the test cells; and
(3) (i) analyzing the nucleic acid for mutations in exon 1 of the RB2/p130 gene, wherein the presence of homozygous mutations at nucleotides 178 or 259 of the RB2/p130 gene indicate that the subject has cancer; or
(ii) when the nucleic acid obtained from the test cells comprises DNA, analyzing the methylation status of the RB2/p130 gene, wherein methylation of at least the region from about nucleotide +287 to about +411 of the RB2/p130 gene indicates that the subject has cancer.

9. The method of claim 8, wherein the nucleic acid obtained from the test cells comprises DNA.

10. The method of claim 8, wherein the nucleic acid obtained from the test cells comprises RNA.

11. The method of claim 8, wherein the nucleic acid is obtained from the test cells is DNA and is analyzed for the methylation status of the RB2/p130 gene, and wherein the region from about nucleotide −95 to about +177 and the region from about nucleotide +167 to about +302 is also methylated.

12. The method of claim 8, wherein the tumor cells are from tumors derived from sarcomas, carcinomas, adenocarcinomas, cancers of neural origin, or hematological neoplasias.

13. The method of claim 8, wherein the tumor cells are from cancers having their origin in the breast; tissues of the male and female urogenital system; lung; tissues of the gastrointestinal system; exocrine glands; tissues of the mouth and esophagus; brain and spinal cord; kidney; pancreas; hepatobiliary system; lymphatic system; smooth and striated muscle; bone and bone marrow; skin; and tissues of the eye.

14. The method of claim 8, wherein the cancer is selected from the group consisting of retinoblasotma, lung cancer, T-lymphoblastoid leukemia, B-lymphoblastoid leukemia, chronic myeloid leukemia, breast carcinoma, colon cancer, ovarian cancer or endometrial cancer.

15. A method of detecting cells which are predisposed to tumorigenesis, comprising:
(1) obtaining a biological sample comprising test cells from a subject, wherein the test cells appear histologically or morphologically normal;
(2) obtaining nucleic acid from the test cells; and
(3) (i) analyzing the nucleic acid for mutations in exon 1 of the RB2/p130 gene, wherein the presence of homozygous mutations at nucleotides 178 or 259 of the RB2/p130 gene indicate that the test cells are predisposed to tumorigenesis; or
(ii) when the nucleic acid obtained from the test cells comprises DNA, analyzing the methylation status of the RB2/p130 gene, wherein methylation of at least the region from about nucleotide +287 to about +411 of the RB2/p130 gene indicates that the test cells are predisposed to tumorigenesis.

16. The method of claim 15, wherein the nucleic acid obtained from the test cells comprises DNA.

17. The method of claim 15, wherein the nucleic acid obtained from the test cells comprises RNA.

18. The method of claim 15, wherein the nucleic acid is obtained from the test cells is DNA and is analyzed for the methylation status of the RB2/p130 gene, and wherein the region from about nucleotide −95 to about +177 and the region from about nucleotide +167 to about +302 is also methylated.

19. The method of claim 15, wherein the biological sample is obtained from tissue of ectodermal, mesodermal or endodermal origin.

20. The method of claim 15, wherein the biological sample is obtained from retinal, lung, ovarian, endometrial, breast or colon tissue.

21. A method of treating cancer or inhibiting tumorigenesis, comprising:
(1) providing a subject who has, or is at risk for developing, cancer, wherein cells of the subject have a homozygous mutation at nucleotides 178 or 259 of the RB2/p130 gene or have methylation of at least the region from about nucleotide +287 to about +411 of the RB2/p130 gene; and
(2) administering an effective amount of a demethylating agent to the subject.

22. A method of inhibiting uncontrolled growth in cells that have a homozygous mutation at nucleotides 178 or 259 of the RB2/p130 gene or have methylation of at least the region from about nucleotide +287 to about +411 of the RB2/p130 gene, comprising the step of contacting the cells with an effective amount of a demethylating agent, such that the methylation status of the RB2/p130 gene in the cells is altered.

23. An isolated nucleic acid sequence encoding a mutant pRb2/p130 protein, comprising a nucleic acid sequence selected from the group consisting of SEQ ID NO: 3, SEQ ID NO: 5 and SEQ ID NO: 7.

24. An isolated nucleic acid encoding a mutant pRb2/p130 protein selected from the group consisting of SEQ ID NO: 4, SEQ ID NO: 6 and SEQ ID NO: 8.

25. An isolated mutant pRb2/p130 protein selected from the group consisting of SEQ ID NO: 4, SEQ ID NO: 6 and SEQ ID NO: 8.

26. An antibody which binds to the isolated mutant pRb2/p130 protein of claim 25 and not to the wild-type pRb2/p130 protein of SEQ ID NO: 2.

27. A method of detecting tumor cells, comprising:
(1) obtaining a biological sample comprising test cells from a subject;
(2) obtaining protein from the test cells; and
(3) analyzing the protein for mutations in pRb2/p130, wherein the presence of a substitution of serine for proline at codon 37 and/or a substitution of proline for alanine at codon 64 of pRB2/p130 indicate that the test cells are tumor cells.

28. A method of detecting cells which are predisposed to tumorigenesis, comprising:
(1) obtaining a biological sample comprising test cells from a subject, wherein the test cells appear histologically or morphologically normal;
(2) obtaining protein from the test cells; and
(3) analyzing the protein for mutations in pRb2/p130, wherein the presence of a substitution of serine for proline at codon 37 and/or a substitution of proline for alanine at codon 64 of pRB2/p130 indicate that the test cells are predisposed to tumorigenesis.

29. A method for detecting sporadic retinoblastoma tumor cells or for diagnosing sporadic retinoblastoma in a subject, comprising the steps of:
(1) obtaining a biological sample comprising test cells from a subject;
(2) obtaining nucleic acid from the test cells; and
(3) analyzing the nucleic acid obtained from the test cells for mutations in exon 12 of the RB2/p130 gene, wherein the presence of a homozygous mutation at nucleotide 1650 of the RB2/p130 gene indicates that the test cells are tumor cells or that the subject has sporadic retinoblastoma.

30. An isolated nucleic acid comprising SEQ ID NO: 9.

31. A method of treating cancer or inhibiting proliferation of tumor cells, comprising inhibiting the binding of ICBP90 to regions of DNA involved in transcriptional regulation of a tumor suppressor gene or other gene involved in mitigating tumorigenesis, such that formation of pRb2/p130 complexes is reduced.

32. A method of treating cancer or inhibiting proliferation of tumor cells, comprising inhibiting the formation of multi-protein transcriptional repressor complexes on a tumor suppressor gene or other gene involved in mitigating tumorigenesis.
